(19) United States
(12) Patent Application Publication
(10) Pub. No.: US 2006/0193866 A1
(43) Pub. Date: Aug. 31, 2006

(54) H pylori antigens

(75) Inventors: Andreas Meinke, Presabaum (AT); Du Min Bui, Vienna (AT); Eszter Nagy, Vienna (AT); Tamas Henics, Taorstrasse (AT)

Correspondence Address: FULBRIGHT & JAWORSKI L.L.P., 600 CONGRESS AVE., SUITE 2400, AUSTIN, TX, 78701, US

(73) Assignee: Intercell AG, Vienna (AT)

(21) Appl. No.: 10/553,928

(22) PCT Filed: Apr. 22, 2004
(86) PCT No.: PCT/EP04/04255

§ 371 Date: Oct. 20, 2005

(30) Foreign Application Priority Data
Apr. 22, 2003 (EP) 034-50097.5

(51) Int. Cl. C07H 21/04; C12P 21/06; A61K 39/40; C12P 21/04; C07K 16/12; C12N 5/06

(52) U.S. Cl. 424/164.1; 435/69.1; 435/70.21; 435/326; 530/388.4; 536/23.53

(57) ABSTRACT

The present invention discloses isolated nucleic acid molecules encoding a hyperimmune serum reactive antigen or a fragment thereof as well as hyperimmune serum reactive antigens or fragments thereof from H. pylori, methods for isolating such antigens and specific uses thereof.

The present invention relates to isolated nucleic acid molecules, which encode antigens for Helicobacter pylori, which are suitable for use in preparation of pharmaceutical medicaments for the prevention and treatment of bacterial infections caused by Helicobacter pylori.

Helicobacter pylori is a Gram-negative, microaerobic, spiral and flagellated bacterium representing the most prevalent human pathogen with nearly half the globe's population infected. Infection most likely occurs in early childhood (<age 10) in most cases and the pathogen specifically colonizes the stomach where it becomes a resident. Colonization lasts for years or even decades but it can persist for life, yet about 70-80% of colonized individuals remain asymptomatic and never develop disease. It is now clear that the prolonged interaction of H. pylori with gastric epithelia is a complex and dynamic process, which leads to chronic acute inflammation of the gastric mucosa and to the development of peptic ulcer disease in 10-20% of the cases. Nearly all duodenal ulcers are caused by H. pylori and, in the stomach ulcers can develop into gastric adenocarcinoma with a frequency of 0.1-4%. The significance of this number is that gastric cancer is the second most common fatal malignancy after lung cancer and within twenty years, it is predicted to be the 8th leading cause of death of any origin worldwide. H. pylori infection is also associated with about 90% of mucosa-associated lymphoid tissue lymphomas (MALT). Patients with H. pylori infection develop high titers of primarily IgG and IgA antibodies but their role in the immune response against the bacterium is not known. Presence of the bacteria within the mucosal epithelium is associated with massive neutrophil infiltration. Considerable evidence exists demonstrating that the H. pylori-induced Th1-biased CD4⁺ T cell response with prominent IFN-γ production might be a strong contributing factor in the outcome of the local immune reaction linked to tissue damage.

H. pylori is inherently equipped with an array of extremely potent factors and mechanisms that enable the pathogen to uniquely adapt to the gastric mucosal environment leading to survival and long-term colonization in humans. However, the known virulence factors are only associated with increased risk of disease and are not absolute. Perhaps the most obvious system what H. pylori developed is its powerful urease enzyme, which by converting urea into ammonia and carbon dioxide allows survival under acidic conditions. Expression of the cytoplasmic apoenzyme is constitutive and its abundance can be as high as 15% of total H. pylori protein. The activity of the enzyme is increased in low pH and the conductance of the inner membrane is also increased for urea under low pH conditions. The produced NH₃ diffuses to the periplasm protecting thereby the bacterium against the extremely acidic environment {Prinz, C. et al., 2003}. The vacuolating cytotoxin product of the vacA gene of H. pylori induces vacuole formation and, thus, perturbation of structure and function in epithelial cells. The vacA gene is present in all strains but its expression varies. It is now known that VacA acts through a Z-type protein tyrosine phosphatase receptor by increasing its tyrosine phosphorylation activity on the G-protein coupled receptor kinase-interactor 1, leading to marked detachment of gastric epithelial cells from their base membrane, a possible mechanism behind H. pylori-induced epithelial cell demise and consequent peptic ulcer formation {Fujikawa, A. et al., 2003}. A major virulence-associated genetic element in H. pylori is the 40 Kbp pathogenicity island, Cag (cytotoxin associated gene) PAI, contained in the majority of strains. The PAI harbors about 30 genes and one gene product, CagA was originally identified in serological studies as an important determinant of disease outcome in H. pylori infected individuals. The Cag PAI contains genes that have close sequence similarities to a type IV secretion system, known to provide a mechanism for direct transfer of bacterial effector proteins into eukaryotic host cells. Not surprising therefore that the CagA protein has been demonstrated as the effector protein that translocates from adherent H. pylori into epithelial cells in vitro. The subsequently phosphorylated CagA rearranges the host cytoskeleton, which then leads to pedestal formation adjacent to the bacteria {Bjorkholm, B. et al., 2003}. Although, it is present in strains expressing VacA, the cagA gene is not linked chromosomally to vacA. Strains with the cagA PAI and the vacA genotype (type I strains) are associated with higher frequency with patients suffering from duodenal ulcer, atrophic gastritis and gastric carcinoma compared with those lacking CagA and VacA (type R strains) {Censini, S. et al., 1996}. Human populations in distinct geographical regions can be differentiated based on genotypic variations located to the right end of the cag PAI {Kersulyte, D. et al., 2000}.

Attachment of H. pylori to gastric epithelium is promoted by a number of factors. The Hpa hemagglutinin binds to sialic acid components of erythrocytes while the babA2-encoded BabA adhesion binds to the histo-blood group antigen Lewis^{b}, present on gastric epithelial cells. babA2-positive strains are more frequently isolated from patients with peptic ulcer disease and gastric carcinoma than type 1 strains and, when compared to type 1 strains lacking babA2, type 1 isolates that also harbor the babA2 gene are more prevalent in patients with atrophic gastritis and intestinal metaplasia. The SabA adhesion protein is shown to associate with a glycoconjugate on the onco-fetal surface antigen sialyl-Le^{x} that is expressed on immature cells of the developing fetal gastric epithelium as well as on rapidly proliferating undifferentiated cells of cancerous and precancerous lesions {Dubreuil, J. et al., 2002}.

Another potent virulence-associated mechanism evolved in H. pylori is its natural competence for transformation together with the pathogen's highest rate of recombination of any known bacterial species. This mechanism makes H. pylori capable of acquiring new genetic material via horizontal gene transfer, a common phenomenon during colonization of an individual and this can result in the generation of novel pathogen subtypes (quasispecies) that exhibit profound changes in virulence markers, such as the cag PAI {Loughlin, M et al., 2003}. Such extreme genetic variability, with any given isolate easily distinguishable from most others by DNA fingerprinting, has also been proposed to account for the expression diversity of many cell surface associated or secreted proteins {Ferrero, R. et al., 2001}. Although, it is not yet clear why only a relatively small portion of the infected population develop clinically manifest disease, the above mentioned pathogen-related factors together with emerging host-specific characteristics, such as IL-1β promoter allele polymorphism, are likely contribute to the complex mechanisms that lie behind H. pylori pathogenicity {Blaser, M., 2000}.

Today, patients diagnosed with H. pylori infection are treated with a combination of one or two antibiotics and a proton pump inhibitor or bismuth. There are a number of standard combinations but re-infection (most likely from parts of the stomach where eradication did not happen) can occur. Current combinational treatment regimes reach 80-90% eradication rates in most cases but since H. pylori strains are emerging with resistance to one or more of the antibiotics that currently comprise any of the treatment combinations, development of new strategies is urgently needed for an effective treatment to prevent or ameliorate H. pylori infections. A vaccine could not only prevent infections by Helicobacter, but more specifically prevent or ameliorate colonization of host tissues, thereby reducing the incidence of gastric atrophy, peptic ulcer disease and gastric cancer. Elimination of severe chronic conditions would be a direct consequence of reducing the incidence of acute infection and carriage of the organism.

A vaccine can contain a whole variety of different antigens. Examples of antigens are whole-killed or attenuated organisms, subfractions of these organisms/tissues, proteins, or, in their most simple form, peptides. Antigens can also be recognized by the immune system in form of glycosylated proteins or peptides and may also be or contain polysaccharides or lipids. Short peptides can be used since for example cytotoxic T-cells (CTL) recognize antigens in form of short usually 8-11 amino acids long peptides in conjunction with major histocompatibility complex (MHC). B-cells can recognize linear epitopes as short as 45 amino acids, as well as three-dimensional structures (conformational epitopes). In order to obtain sustained, antigen-specific immune responses, adjuvants need to trigger immune cascades that involve all cells of the immune system necessary. Primarily, adjuvants are acting, but are not restricted in their mode of action, on so-called antigen presenting cells (APCs). These cells usually first encounter the antigen(s) followed by presentation of processed or unmodified antigen to immune effector cells. Intermediate cell types may also be involved. Only effector cells with the appropriate specificity are activated in a productive immune response. The adjuvant may also locally retain antigens and co-injected other factors. In addition the adjuvant may act as a chemoattractant for other immune cells or may act locally and/or systemically as a stimulating agent for the immune system.

Attempts to develop a Helicobacter vaccine have focused mainly on whole-cell and attenuated or subunit vaccine approaches. The initial “proof of principle” studies to generate an H. pylori vaccine were performed using inactivated whole-cell preparations and cholera toxin as a mucosal adjuvant. Although, such vaccines were highly effective in inducing protective immunity against gastric infection in mice, their safety and licensing as well as difficulties in producing H. pylori preparations in vitro in large scale eliminated them from human trials {Ferrero, R. et al., 2001}; {Sutton, P., 2001}. For second generation subunit vaccines, candidate antigens were identified by empirical approaches. The selection criteria for these antigens were linked to known or suspected roles of the proteins in bacterial virulence. Such candidates include the urease holenzyme and its subunits, UreA and UreB, heat shock protein homologues of the chaperonins GroEL and GroES, the VacA cytotoxin and catalase (KatA) ({Prinz, C. et al., 2003}; {Svennerholm, A., 2003} and references therein). Another set of candidate proteins were identified in subsequent studies on the basis of their immunoreactivity in in vitro assays. H. pylori genomic expression libraries were screened with antibodies from mice that had been immunized with H. pylori whole cell sonicates or outer membrane vesicles in the presence of cholera toxin. Antigenic proteins were purified from selected E. coli clones and their identity determined by N-terminal sequencing. Among known antigens, such as UreA, UreB, the GroEL homologue and Lpp20 lipoprotein, four previously uncharacterized proteins were also identified. One had homology to L7/L12 ribosomal proteins and the rest were of unknown function. A similar screening strategy combined with a chimeric fusion technique confirmed the Lpp20 protein as a vaccine candidate antigen {Oliaro, J. et al., 2000}. There are other proteins under consideration for vaccine development that are based on recent identifications employing multiparameter selection criteria. These include an Hpa homologue (HP0410) and a novel protein of unknown function (HP0231), both with high protective efficacy {Sabarth, N. et al., 2002}. Despite the benefits of both prophylactic and therapeutic vaccination in animals as demonstrated in several studies, bacterial eradication (sterilizing immunity) has not been described in humans.

Since the above mentioned identification methods are either empirical or limited to a specific selection criterion, there is a demand to identify additional relevant antigens of H. pylori using an efficient and comprehensive identification and validation technology.

The present inventors have developed a method for identification, isolation and production of hyperimmune serum reactive antigens from a specific pathogen, especially from Staphylococcus aureus and Staphylococcus epidermidis (WO 02/059148). However, given the differences in biological property, pathogenic potency and genetic background, Helicobacter pylori is distinctive from Staphylococcus strains. Importantly, the selection of sera for the identification of antigens from H. pylori is different from that applied to the S. aureus screens.

Three major types of human sera were collected for this purpose. First, healthy adults below <45 years of age were tested for H. pylori-specific IgG and IgA serum antibody levels by ELISA using total bacterial lysate and culture supernatant proteins. High titer individuals were interviewed and seleted based on the absence of medical history, symptoms or complains related to H. pylori diseases. Based on correlative data, protective (colonization neutralizing) antibodies are likely to be present in exposed individuals who are not carriers of H. pylori or not susceptible to disease casued by H. pylori. High titer sera from symptom-free healthy adults were included in the genomic based antigen identification. This approach for selection of human sera is basically very different from that used for S. aureus, where carriage or noncarriage state cannot be associated with antibody levels.

Second, serum samples from patients with gastric cancer were characterized for anti-H. pylori antibody titers using ELISA and high titer sera were selected for the screens. The third group of serum samples was obtained from individuals with duodenal ulcer and high titer sera determined by ELISA were selected for the screens.

The genomes of the two bacterial species H. pylori and S. aureus by itself show a number of important differences. The genome of H. pylori contains approximately 1.65 Mb sequence information, while S. aureus harbours about 2.85 Mb. They have an average GC content of 39 and 33%, respectively. In addition, the two bacterial species require different growth conditions and media for propagation. While H. pylori is a strictly human pathogen, S. aureus can also be found infecting a range of warm-blooded animals. A list of the most important diseases, which can be inflicted by the two pathogens, is presented below. S. aureus causes mainly nosocomial, opportunistic infections: impetigo, folliculitis, abscesses, boils, infected lacerations, endocarditis, meningitis, septic arthritis, pneumonia, osteomyelitis, scalded skin syndrome (SSS), toxic shock syndrome. H. pylori causes likely community aquired gastro-intestinal infections: self limiting traveler's diarrhea, corpus predominant or pangastritis, peptic ulcer disease (stomach and duodenum), gastric cancer (adenocarcinoma), chronic atrophic gastritis (CAG) and MALT (mucosa-associated lymphoid tissue, non-Hodgkin's type B cell lymphoma).

The problem underlying the present invention was to provide means for the development of medicaments such as vaccines against H. pylori infection. Particularly, the problem was to provide an efficient, relevant and comprehensive set of nucleic acid molecules or antigens from H. pylori that can be used for the manufacture of said medicaments.

Therefore, the present invention provides an isolated nucleic acid molecule encoding a hyperimmune serum reactive antigen or a fragment thereof comprising a nucleic acid sequence, which is selected from the group consisting of:
a) a nucleic acid molecule having at least 70% sequence identity to a nucleic acid molecule selected from Seq ID No 3-4, 16, 19-21, 28-29, 33-38, 41-42, 44, 48-52, 55, 57-58, 61, 63, 65, 67-68, 72, 74-75, 81, 84, 91, 94, 96-97, 101, 105-108, 112, 115-117, 119, 123-178.
b) a nucleic acid molecule which is complementary to the nucleic acid molecule of a),
c) a nucleic acid molecule comprising at least 15 sequential bases of the nucleic acid molecule of a) or b)
d) a nucleic acid molecule which anneals under stringent hybridisation conditions to the nucleic acid molecule of a), b), or c)
e) a nucleic acid molecule which, but for the degeneracy of the genetic code, would hybridise to the nucleic acid molecule defined in a), b), c) or d).

According to a preferred embodiment of the present invention the sequence identity is at least 80%, preferably at least 95%, especially 100%.

Furthermore, the present invention provides an isolated nucleic acid molecule encoding a hyperimmune serum reactive antigen or a fragment thereof comprising a nucleic acid sequence selected from the group consisting of
a) a nucleic acid molecule having at least 96% sequence identity to a nucleic acid molecule selected from Seq ID No 8-10, 13-15, 17-18, 24, 27, 32, 39-40, 45-47, 56, 59, 62, 69-70, 73, 77, 79, 82, 85-86, 88, 90, 103, 109-110, 114, 121,
b) a nucleic acid molecule which is complementary to the nucleic acid molecule of a),
c) a nucleic acid molecule comprising at least 15 sequential bases of the nucleic acid molecule of a) or b)
d) a nucleic acid molecule which anneals under stringent hybridisation conditions to the nucleic acid molecule of a), b) or c),
e) a nucleic acid molecule which, but for the degeneracy of the genetic code, would hybridise to the nucleic acid defined in a), b), c) or d).

According to another aspect, the present invention provides an isolated nucleic acid molecule comprising a nucleic acid sequence selected from the group consisting of
a) a nucleic acid molecule selected from Seq ID No 5, 7, 30-31, 53, 60, 66, 76, 83, 87, 92, 99, 120,
b) a nucleic acid molecule which is complementary to the nucleic acid of a),
c) a nucleic acid molecule which, but for the degeneracy of the genetic code, would hybridise to the nucleic acid defined in a), b), c) or d).

Preferably, the nucleic acid molecule is DNA or RNA.

According to a preferred embodiment of the present invention, the nucleic acid molecule is isolated from a genomic DNA, especially from a H. pylori genomic DNA.

According to the present invention a vector comprising a nucleic acid molecule according to any of the aspects of the present invention is provided.

In a preferred embodiment the vector is adapted for recombinant expression of the hyperimmune serum reactive antigens or fragments thereof encoded by the nucleic acid molecule according to the present invention.

The present invention also provides a host cell comprising the vector according to the present invention.

According to another aspect the present invention further provides a hyperimmune serum-reactive antigen comprising an amino acid sequence being encoded by a nucleic acid molecule according to the present invention.

In a preferred embodiment the amino acid sequence (polypeptide) is selected from the group consisting of Seq ID No 181-182, 194, 197-199, 206-207, 211-216, 219-220, 222, 226-230, 233, 235-236, 239, 241, 243, 245-246, 250, 252-253, 259, 262, 269, 272, 274-275, 279, 283-286, 290, 293-295, 297, 301-356.

In another preferred embodiment the amino acid sequence (polypeptide) is selected from the group consisting of Seq ID No 186-188, 191-193, 195-196, 202, 205, 210, 217-218, 223-225, 234, 237, 240, 247-248, 251, 255, 257, 260, 263-264, 266, 268, 281, 287-288, 292, 299.

In a further preferred embodiment the amino acid sequence (polypeptide) is selected from the group consisting of Seq ID No 183, 185, 208-209, 231, 238, 244, 254, 261, 265, 270, 277, 298.

According to a further aspect the present invention provides fragments of hyperimmune serum-reactive antigens selected from the group consisting of peptides comprising amino acid sequences of column “predicted immunogenic aa” and “location of identified immunogenic region” of Table 1, the serum reactive epitope of Table 3 especially peptides comprising amino acids 63-91, 95-101, 110-116, 134-148, 150-156, 158-164, 188-193, 197-209, 226-241, 247-254, 291-297, 312-319, 338-346, 351-358, 366-378, 404-410, 420-438, 448-454, 465-473, 482-488, 490-498, 503-510, 512-519, 531-543, 547-554, 568-575, 589-604, 610-631 and 239-308 of Seq ID No 179; 16-29, 35-47, 50-68, 70-79, 91-101, 143-149, 158-163, 185-191, 196-206, 215-224, 230-237, 244-251, 258-278, 290-311, 319-325, 338-351, 365-385, 396-429, 445-454, 458-466, 491-499, 501-521, 17-79 and 218-233 of Seq ID No 180; 4-10, 16-41, 46-66, 77-84, 91-97, 102-118, 125-144, 187-200, 202-214, 245-253, 255-261, 286-295, 300-330, 335-342, 350-361, 363-381, 385-392, 396-416, 435-450 and 460-470 of Seq ID No 181; 11-19, 27-48, 52-59, 77-82, 84-107, 118-125, 127-154, 178-183, 192-209, 215-221, 286-295, 302-313, 350-357, 402-415, 417-431, 453-463, 465-493 and 313-331 of Seq ID No 182; 19-26, 30-43, 47-55, 63-68, 72-80, 97-104, 107-119, 129-146, 160-175, 194-216, 231-251, 254-260 and 26-43 of Seq ID No 183; 7-13, 29-37, 65-81, 110-120, 123-131, 135-152, 230-249, 254-260, 284-290, 292-299, 317-326, 329-336, 403-444, 452-458, 466-477, 490-498, 510-519, 541-550, 557-566 and 533-567 of Seq ID No 184; 5-47, 71-77, 79-86, 89-95, 120-126, 137-144, 176-181, 184-196, 202-208, 211-232, 236-282, 301-313, 317-325, 341-347, 353-384, 394-400, 412-433, 436-443 and 59-75 of Seq ID No 185; 4-18, 22-38, 59-69, 106-112, 116-130, 138-149, 156-170, 175-197, 200-214, 216-223, 233-244, 255-261, 266-276, 279-286, 325-333, 342-348, 366-399, 402-420, 429-441, 1-104 and 130-147 of Seq ID No 186; 50-58, 69-95, 97-113, 131-136, 157-163, 170-175, 188-212, 220-226, 254-259, 265-277, 283-289, 297-308, 311-318, 347-358, 360-369, 378-401, 416-421, 440-450, 454-462, 470-476, 493-502, 506-514, 536-567, 585-590, 598-607, 613-618, 653-659 and 35-46 of Seq ID No 187; 16-29, 32-60, 65-87, 89-123, 128-134, 137-158, 162-173, 178-196, 210-216, 218-228 and 206-225 of Seq ID No 188; 10-20, 26-35, 51-64, 86-91, 94-100, 113-122, 154-160, 185-191, 193-201, 211-217, 225-230, 237-246, 251-257, 298-304, 306-312, 316-328, 340-348, 357-389, 391-397, 415-421, 449-456, 458-471, 488-495, 502-511, 24-55 and 236-341 of Seq ID No 189; 5-22, 41-51, 87-93, 114-122, 127-136, 150-156, 158-166, 223-233, 245-263, 291-296, 9-126 and 127-285 of Seq ID No 190; 30-43, 46-56, 61-70, 72-83, 85-93, 103-113, 119-125, 151-166, 179-191, 212-218, 225-231, 236-243, 262-267, 291-307, 331-344, 349-355, 366-372, 380-386, 414-422, 428-447, 459-464, 469-478, 507-519, 525-544, 563-569, 576-590, 620-626, 633-643, 654-659, 665-671, 684-707, 717-723, 725-733, 747-779, 782-801 and 347-361 of Seq ID No 191; 4-12, 14-26, 37-80, 107-115, 133-139, 144-150, 154-165, 173-180, 191-199, 205-211, 221-231, 237-244, 254-284, 307-340, 342-353, 360-368, 370-380, 479-493, 495-503, 509-522, 525-536, 539-547, 554-560, 565-573, 578-583, 7-23 and 465-479 of Seq ID No 192; 4-17, 47-55, 76-83, 85-100, 104-112, 117-123, 126-135, 142-148, 156-167, 174-182, 267-273 and 258-283 of Seq ID No 193; 8-32, 36-42, 65-88, 102-108, 112-140, 147-163, 170-179, 183-193 and 117-124 of Seq ID No 194; 12-18, 45-50, 62-77, 82-95, 99-113, 115-123, 125-147, 155-177, 187-209, 211-223, 244-253, 259-270, 278-297, 302-307, 311-318, 329-334, 350-356, 359-365, 390-400, 402-413 and 333-350 of Seq ID No 195; 4-13, 15-27, 30-46, 53-58, 68-74, 82-95, 115-126, 134-139, 148-153, 159-176, 182-199, 201-217, 220-225, 227-235, 237-248, 253-266, 300-315, 322-336, 390-396, 412-426, 438-445, 448-459, 477-484, 502-508, 515-527, 529-537, 553-568, 643-651, 658-667, 690-703 and 376-400 of Seq ID No 196; 4-10, 24-32, 38-55, 59-67, 70-77, 80-87, 89-97, 123-129, 134-151, 166-172, 178-189, 191-216, 218-235, 245-259, 271-315, 326-339, 341-360 and 73-94 of Seq ID No 197; 13-25, 31-3, 43-57, 79-85, 92-99, 106-112, 117-128, 130-139, 146-158, 160-175, 194-204, 211-222, 225-232, 234-242, 263-270, 278-292, 299-320, 322-333 and 240-256 of Seq ID No 198; 4-17, 55-63, 66-101, 109-131, 135-143, 145-151, 155-161, 164-170, 177-185, 192-198, 213-218, 223-238, 246-256, 258-268, 273-283, 309-314, 322-328 and 195-221 of Seq ID No 199; 13-24, 31-39, 41-50, 63-69, 90-96, 104-109, 116-141, 148-153, 161-167, 173-178, 190-209, 253-258, 265-272, 279-289, 295-312, 317-343, 355-366, 376-389, 400-407, 430-451, 453-464, 466-472, 487-493, 499-505, 523-538, 554-559, 568-579, 584-01 and 344-363 of Seq ID No 200; 5-22, 30-36, 53-59, 61-70, 82-92, 99-106, 120-131, 135-148, 154-167, 169-183, 187-199, 204-212, 231-247 and 111-249 of Seq ID No 201; 17-36, 40-66, 71-144, 148-171, 173-191, 199-214, 220-252, 265-272, 278-288, 298-333, 342-385 and 287-307 of Seq ID No 202; 4-16, 22-28, 30-36, 42-48, 95-116, 154-162, 164-174, 239-252, 258-263, 273-285, 306-313, 323-333, 341-357, 363-369, 372-379, 395-401, 430-436, 438-453, 464-480, 33-44, 233-258 and 349-369 of Seq ID No 203; 4-21, 30-37, 46-53, 59-68, 80-92, 98-104, 118-143, 150-160, 165-185, 187-200, 204-211, 224-236, 241-246, 252-258, 271-280, 288-294, 311-320, 335-341 and 191-350 of Seq ID No 204; 4-16, 37-59, 64-70, 79-87, 93-102, 107-127, 143-165, 172-188, 197-204, 207-218, 221-227, 242-248, 258-277, 289-296, 298-316, 332-338, 344-365, 367-373, 375-382, 400-408, 415-425, 438-446 and 235-250 of Seq ID No 205; 4-37, 39-66, 84-98, 101-127, 140-149, 157-163, 166-172, 175-182, 184-193, 203-208, 215-232, 234-247, 250-299, 303-345 and 183-204 of Seq ID No 206; 10-20, 41-61, 73-87, 112-141, 176-192, 194-201, 205-222, 230-237, 257-264, 276-282, 284-310, 312-318, 330-337, 349-357 and 304-328 of Seq ID No 207; 4-31, 42-103, 105-113, 121-153, 160-181, 188-196, 210-226, 231-264, 272-287, 297-304, 328-336 and 304-318 of Seq ID No 208; 21-43, 46-52, 54-70, 72-79, 94-107, 133-141, 160-166, 217-253, 311-317, 359-365, 374-381, 390-395, 434-440, 488-494, 497-502, 511-522, 554-563, 565-574, 577-585, 591-598, 601-606, 617-625, 633-643, 658-664, 676-682, 694-702, 710-719, 754-760, 782-788, 802-808, 916-921, 942-948, 955-964, 973-979, 992-998, 1006-1011, 1016-1023, 1030-1038, 1046-1053, 1059-1066, 1088-1098, 1119-1126, 1129-1135, 1156-1171, 1173-1181, 1202-1210, 1255-1261, 1268-1280, 1295-1310, 1312-1320, 1375-1381, 1406-1417, 1450-1471, 1478-1492, 1498-1506, 1569-1578, 1603-1608, 1611-1624, 1648-1655, 1663-1670, 1680-1698, 1702-1707, 1713-1719, 1737-1742, 1747-1753, 1762-1769, 1771-1785, 1790-1804, 1811-1818, 1830-1836, 1838-1852, 1874-1886, 1893-1899, 1902-1909, 1942-1948, 1952-1962, 1980-1986, 2001-2017, 2020-2028, 2042-2050, 2052-2068, 2074-2079, 2083-2095, 2107-2113, 2147-2155, 2177-2194, 2203-2211, 2236-2241, 2251-2258, 2267-2274, 2285-2292, 2314-2328, 2330-2340, 2358-2365, 2390-2401, 2408-2418, 2432-2453, 2463-2476, 2486-2507, 2528-2537, 2540-2548, 2552-2558, 2568-2576, 2596-2601, 2610-2622, 2629-2638, 2653-2669, 2718-2727, 2749-2767, 2777-2784, 2789-2795, 2806-2815, 2817-2824, 2835-2843, 2847-2854, 2860-2881, 511-523, 612-630 and 1790-1803 of Seq ID No 209; 4-54, 61-68, 72-82, 86-93, 100-108, 115-130, 147-154, 187-194, 196-207, 224-229, 236-251, 275-287 and 96-109 of Seq ID No 210; 31-39, 62-69, 91-101, 158-172, 175-180, 186-193, 201-208, 210-223, 243-250, 273-286, 293-299, 319-325, 343-354, 356-365, 368-384, 414-435, 471-491, 512-518, 550-556, 567-581, 584-589, 633-639, 680-692, 697-708, 716-721, 747-754, 779-786, 810-816 and 366-503 of Seq ID No 211; 5-20, 22-48, 57-65, 96-101, 111-122, 130-145, 154-164, 170-181, 193-199, 201-216, 224-241, 244-262, 281-323, 342-351, 359-367, 369-396, 406-416, 424-433, 450-456, 485-491, 493-499, 501-515, 517-535 and 289-305 of Seq ID No 212; 4-17, 22-44, 53-60, 66-83, 87-94, 101-106, 110-116, 131-137, 148-183, 189-207, 209-215, 233-242, 251-262, 264-272, 290-296, 308-327, 359-373, 375-380, 397-405, 415-420, 426-433, 444-475, 478-484, 529-536, 548-558 and 106-126 of Seq ID No 213; 4-38, 42-50, 58-64, 72-81, 92-118, 140-146, 157-165, 172-192, 198-204, 208-216, 227-234, 238-258, 271-278, 288-293, 311-322, 327, 346, 357-370, 375-383, 395-409, 411-417, 425-432, 436-445, 109-129 and 370-380 of Seq ID No 214; 23-30, 36-49, 52-64, 86-94, 97-104, 121-129, 257-272, 279-286, 288-294, 307-327, 334-340, 369-375, 377-386, 406-412, 418-423, 430-438, 441-447, 459-465, 469-476, 482-488, 510-546, 550-580, 584-622, 638-645, 653-659, 675-63, 692-705, 723-731, 752-761, 788-795 and 572 of Seq ID No 215; 11-33, 36-46, 88-104, 116-126, 134-170, 189-195, 199-217, 225-250, 255-261, 266-273, 280-291, 296-313, 334-341, 343-349, 354-360, 362-369, 373-380, 387-401, 406-420 and 259-273 of Seq ID No 216; 9-14, 28-44, 57-64, 72-79, 86-93, 104-111, 116-126, 142-150, 159-164 and 61-86 of Seq ID No 217; 10-17, 26-33, 43-61, 69-95, 101-107, 109-125, 129-135, 137-144, 147-153, 158-169, 177-187, 209-219, 221-232, 235-247, 261-268, 271-282, 296-302, 306-347, 355-362, 364-379, 386-399, 409-418, 424-442, 451-460, 467-479, 490-498 and 60-74 of Seq ID No 218; 8-14, 20-31, 65-84, 94-99, 154-179, 193-207, 238-253 and 96-118 of Seq ID No 219; 4-24, 30-44, 47-62, 84-93, 108-116, 124-133, 136-141, 201-209, 217-223, 228-235, 238-245, 247-270, 275-285, 290-314, 328-338, 342-349, 353-365, 375-383, 386-392, 394-402, 417-427, 443-459, 465-481, 492-514, 516-524, 550-566, 602-617, 630-639, 666-676, 687-693, 719-730, 747-753, 783-790, 799-816, 824-831, 837-842 and 167-189 of Seq ID No 220; 6-15, 18-28, 58-66, 84-101, 106-129, 136-151, 154-165, 182-203, 205-211, 214-220, 222-228, 233-240, 251-260, 270-277, 284-291, 306-315, 322-328, 363-369, 378-388, 392-405, 443-452, 495-501, 512-523, 574-583 and 362-375 of Seq ID No 221; 5-25, 27-34, 47-59, 64-70, 76-86, 145-158, 166-183, 189-202, 217-231, 235-242, 260-270, 278-309 and 1-102 of Seq ID No 222; 4-19, 24-76, 78-83, 90-99, 102-109, 114-122, 137-147, 154-174, 177-188, 203-212, 217-223, 227-239 and 226-325 of Seq ID No 223; 7-37, 71-90, 94-109, 117-128, 141-153, 179-192, 199-206, 225-231, 237-243, 258-264 and 40-51 of Seq ID No 224; 13-19, 25-30, 46-59, 75-91, 101-107, 114-124, 129-135, 137-145, 160-167, 171-179, 187-194, 209-215, 217-222, 229-239, 243-249, 257-265, 269-275, 299-308, 310-327 and 282-300 of Seq ID No 225; 86-100, 216-230, 342-369, 382-388, 424-430, 438-445, 452-458, 488-494, 501-518, 554-560, 568-574, 584-592, 603-609, 611-629, 639-645, 652-661, 669-699, 708-714, 726-738, 747-753, 763-775, 785-791, 794-807, 815-824, 826-845, 854-860, 863-868, 870-883, 892-898, 901-906, 909-921, 930-937, 946-959, 968-974, 977-990, 998-1007, 1009-1027, 1037-1043, 1046-1051, 1053-1066, 1075-1081, 1084-1089, 1092-1103, 1113-1119, 1122-1135, 1143-1152, 1154-1172, 1182-1188, 1191-1196, 1200-1210, 1220-1226, 1229-1235, 1237-1249, 1259-1265, 1268-1281, 1289-1298, 1305-1318, 1328-1334, 1337-1343, 1345-1357, 1367-1373, 1390-1396, 1405-1411, 1418-1423, 1426-1435, 1445-1455, 1474-1483, 1493-1500, 1505-1512, 1517-1524, 1538-1544, 1568-1578, 1595-1601, 1674-1682, 1687-1720, 1728-1736, 1738-1744, 1754-1761, 1764-1774, 1798-1824, 1836-1842, 1886-1893, 1895-1903, 366-781, 782-1518 and 1731-1747 of Seq ID No 226; 4-17, 20-39, 46-55, 60-66, 102-110, 114-122, 125-131, 161-167, 172-178, 185-190, 195-202, 218-232, 236-252, 264-291, 293-302, 309-315, 324-339 and 169-381 of Seq ID No 227; 5-10, 13-40, 42-53, 69-75, 83-89, 120-135, 150-161, 174-190, 203-225, 229-247, 257-287, 318-348 and 30-200 of Seq ID No 228; 7-19, 43-53, 64-72, 124-139, 52-84 and 120-131 of Seq ID No 229; 12-19, 39-48, 58-100, 117-123, 154-162, 164-187, 189-195, 202-216, 218-235, 241-246, 262-278, 315-328, 333-347, 354-366, 372-379, 391-405, 422-429, 431-442, 444-450, 458-466, 478-485, 494-501, 504-510, 520-535, 573-580, 589-598, 615-625, 666-676, 686-698, 722-729, 737-746, 756-767, 787-796, 805-816, 824-829, 833-848, 856-864, 866-876, 879-886, 898-904, 918-924, 927-934, 941-960, 967-978 and 561-575 of Seq ID No 230; 11-29, 49-55, 70-77, 84-100, 102-112, 148-155, 160-177, 181-204 and 1-104 of Seq ID No 231; 27-44, 64-71, 122-133, 151-156, 164-178, 214-220, 226-232, 235-244, 253-262, 282-288, 294-310, 317-325, 350-356, 362-368, 376-383, 438-443, 449-454, 459-464, 492-498, 500-511, 529-535, 538-546, 567-573, 597-603, 660-665, 674-679, 724-734, 763-769, 773-784, 791-801, 807-815, 821-826, 840-848, 863-868, 897-902, 908-928, 932-953, 956-975, 980-987, 990-996, 1012-1018, 1042-1063, 1095-1116, 1149-1157, 1160-1167, 110-357, 358-501 and 502-1161 of Seq ID No 232; 4-21, 64-71, 73-84, 128-138, 144-162, 203-217, 240-263, 288-298, 300-308, 310-317, 325-351, 369-380, 391-411 and 330-345 of Seq ID No 233; 5-11, 25-31, 39-48, 51-79, 89-98, 100-122, 135-148, 166-201, 203-227, 230-250, 254-260, 266-272, 274-282, 299-305, 328-337 and 31-4 of Seq ID No 234; 12-23, 29-48, 51-60, 66-72, 75-81, 83-93, 103-115, 133-148, 168-174, 195-204, 222-229, 231-240, 242-251, 270-280, 286-305, 322-344, 349-360, 364-370, 378-400, 421-441, 448-484, 486-493, 495-501, 504-534, 547-561, 567-590, 597-607, 621-635, 643-649, 658-685, 688-694, 702-711, 717-731, 737-742, 759-765, 767-772, 776-786, 803-809, 815-825, 854-908, 910-919, 923-930, 942-948, 961-975, 994-1014 and 915-940 of Seq ID No 235; 4-9, 32-47, 51-61, 75-96, 139-191 and 1-124 of Seq ID No 236; 4-13, 17-38, 43-49, 55-76, 88-95, 110-121, 128-146, 151-157, 162-214, 222-240, 243-249, 251-273, 275-281, 292-298, 300-309, 312-320, 322-331, 355-369, 376-408, 446-460, 471-482, 485-509 and 191-203 of Seq ID No 237; 4-21, 72-82, 89-103, 106-115, 118-124, 140-146, 174-184, 191-200, 204-213, 218-224, 261-266, 282-293, 299-309, 311-340, 342-358, 362-372, 381-389, 391-402, 413-421, 438-447, 457-464, 470-478, 501-507, 545-560, 578-624, 631-641, 658-670, 680-689, 717-738, 753-759, 795-805, 816-822, 830-838, 842-848, 869-881, 892-898, 33-51 and 818-835 of Seq ID No 238; 4-21, 79-85, 156-177, 183-188, 206-214, 243-249, 261-269, 287-292, 315-322, 334-345, 360-366, 374-390, 402-411, 37-97 and 260-399 of Seq ID No 239; 4-9, 19-54, 58-78, 97-104, 111-120, 126-134, 137-145, 163-173, 178-188, 193-203, 211-224, 246-286, 288-324, 337-346, 355-362, 374-390, 392-398, 409-417 and 240-249 of Seq ID No 240; 5-12, 14-31, 35-41, 43-61, 82-92, 97-105, 134-145, 155-166, 184-203, 215-223, 225-251, 272-279, 281-306, 310-345, 358-418, 435-473, 482-490, 525-532, 538-547, 549-563, 578-604, 613-639 and 144-154 of Seq ID No 241; 53-59, 64-72, 74-100, 133-152, 154-172, 176-181, 207-214, 225-238, 275-297, 304-310, 331-340, 362-367, 384-395, 403-410, 437-443, 448-456, 482-490, 579-597, 602-610, 625-630, 633-651, 699-707, 709-715, 734-743, 750-762 and 544-685 of Seq ID No 242; 12-18, 22-40, 45-83, 89-97, 103-109, 147-153, 159-173, 195-204, 210-219, 243-253, 259-265, 273-282, 303-309, 315-325, 332-340, 346-358, 362-37, 377-390, 393-402, 418-426, 447-455, 467-480, 505-512, 514-525, 548-561, 566-576, 584-596, 619-626, 638-645, 649-659, 661-680, 699-708, 714-720, 753-759, 766-772, 775-781, 801-808, 202-218, 282-299, 339-350 and 617-628 of Seq ID No 243; 5-33, 52-62, 87-101, 111-135, 137-143, 145-152, 190-202, 209-221, 233-245, 253-270 and 151-215 of Seq ID No 244; 19-29, 32-39, 42-48, 75-94, 124-135, 137-145, 152-160, 176-182, 193-203, 215-236, 266-273, 275-291, 297-306, 311-319, 322-342, 348-360, 369-378, 394-401 and 48-64 of Seq ID No 245; 4-11, 13-33, 36-43, 53-63, 65-80, 112-129, 134-141, 143-155, 157-168, 178-188, 191-199, 201-207, 215-229, 242-255, 263-270, 283-315, 320-329, 333-338, 340-349, 412-426, 465-478, 455-490, 498-512, 540-554 and 390-516 of Seq ID No 246; 4-18, 23-32, 41-47, 54-70, 88-99, 104-111, 118-138, 143-148, 150-162, 168-175, 181-188, 203-211, 214-220, 227-245, 251-268, 275-281, 287-296, 323-333 and 1-90 of Seq ID No 247; 8-34, 38-49, 72-83, 85-91, 94-104, 112-125, 134-142, 148-168, 181-189, 191-198, 202-214, 222-233, 242-254, 256-262, 273-278, 287-294, 314-325 and 141-159 of Seq ID No 248; 4-24, 30-36, 47-75, 82-105, 124-134, 151-157, 192-202, 208-214, 219-226, 234-247, 285-290, 318-324, 332-340, 343-349, 380-386, 453-462, 472-478, 484-501, 531-540, 550-557, 604-612, 620-625, 642-648, 652-671, 64-84, 93-180 and 181-446 of Seq ID No 249; 12-18, 24-32, 68-75, 77-83, 96-101, 109-116, 129-136, 152-164, 175-184, 190-199, 206-215, 224-233, 241-250, 258-264, 273-292, 302-312, 319-331, 334-346, 348-368, 387-395, 408-416, 420-429, 437-452 and 364-374 of Seq ID No 250; 11-28, 36-52, 60-67, 74-79, 108-116 and 61-76 of Seq ID No 251; 20-27, 38-49, 69-74, 84-107, 138-145, 161-168, 179-195, 210-226, 228-252, 267-281, 283-296, 305-311, 333-340, 342-356, 361-372, 380-399, 401-414, 458-466, 475-481, 492-507, 515-520 and 146-160 of Seq ID No 252; 43-61, 68-74, 76-90, 120-128, 130-149, 156-161, 164-182, 206-234, 242-252, 269-274, 291-304, 332-345, 349-355, 360-371, 374-388, 434-440, 447-453, 459-465, 469-496, 504-522 and 261-285 of Seq ID No 253; 4-17, 24-30, 37-49, 87-98, 118-124, 126-136, 144-171, 176-188, 206-214, 216-228, 233-240, 246-252, 262-271, 277-297, 307-330, 333-342, 346-352, 355-361, 368-386, 391-400, 413-420, 474-480 and 401-427 of Seq ID No 254; 15-26, 31-46, 51-72, 80-93, 96-109, 131-137, 150-158, 179-185, 189-209, 211-219, 221-234, 241-247, 255-262, 265-271, 283-288 and 173-190 of Seq ID No 255; 28-37, 39-45, 51-58, 77-84, 89-97, 132-148, 171-180, 199-205, 212-218, 220-226, 257-265, 273-300, 307-327, 334-340, 344-365, 385-390, 402-408, 426-436, 450-468, 476-485 and 425-497 of Seq ID No 256; 4-25, 70-76, 80-88, 90-100, 120-128, 162-169, 183-203, 261-277, 279-289, 291-297, 302-308, 321-327, 339-353, 358-377, 392-401, 404-410, 414-422, 443-450, 456-461, 470-488, 490-497, 510-535, 570-611, 618-630, 639-647, 649-660, 668-690, 702-716, 718-724, 737-747, 750-764 and 497-509 of Seq ID No 257; 12-48, 50-64, 99-108, 216-223, 235-241, 244-254, 262-274, 287-293, 310-316, 320-326, 361-366, 377-383, 390-395, 408-414, 418-425, 438-444, 462-469, 494-505, 524-530, 536-547, 551-566, 592-598, 601-613, 678-685, 687-695, 709-717, 727-737, 751-757, 760-765, 772-778, 782-788, 801-807, 822-830, 859-868, 870-878, 884-890, 898-903, 909-919, 953-969, 973-980, 990-1000, 1002-1019, 1041-1047, 1059-1065, 1090-1095, 1116-1127, 1130-1139, 1143-1149, 1151-1168, 1178-1183, 1188-1195, 1197-1209, 1213-1220, 1226-1234, 1236-1247, 1255-1274, 1276-1282, 76-100, 270-284, 309-438, 493-505, 786-942 and 947-967 of Seq ID No 258; 4-9, 24-34, 46-95, 97-109, 119-130 and 138-156 of Seq ID No 259; 9-26, 28-35, 43-53, 55-68, 83-92, 99-105, 110-135, 139-149, 157-162, 164-170, 173-183, 193-208, 210-230, 239-245, 253-259, 263-271, 293-305, 310-320, 322-331, 336-343, 351-364, 367-376, 92-107 and 154-173 of Seq ID No 260; 19-39, 52-62, 108-117, 145-152, 160-168, 194-203, 229-240, 252-268, 280-287, 308-316, 333-339, 383-390, 403-412, 414-424, 438-445, 464-472, 479-484, 489-505, 510-526 and 247-260 of Seq ID No 261; 5-17, 25-52, 60-77, 105-113, 118-125, 162-167, 228-234, 272-279, 328-334, 341-357, 381-395, 400-406, 512-518, 557-569, 586-592, 645-651, 690-695, 701-709, 720-726, 733-743, 751-758, 781-786, 879-886, 929-934, 939-944, 952-960, 965-975, 994-1001, 1039-1045, 1102-1109, 1164-1181, 1198-1206, 1223-1229, 1253-1259, 1283-1292, 1312-1317, 1339-1349, 1360-1370, 1389-1398, 1400-1412, 1452-1465, 1470-1484, 1490-1497, 1519-1525, 1554-1564, 1578-1591, 1623-1636, 1638-1646, 1669-1679, 1685-1697, 1704-1711, 1713-1720, 1730-1736, 1738-1749, 1756-1764, 1778-1786, 1796-1803, 1817-1826, 1849-1866, 1975-1993, 2017-2032, 2044-2053, 2070-2086, 2091-2109, 2116-2127, 2156-2167, 2182-2188, 2197-2202, 2244-2252, 2281-2287, 2290-2307, 2350-2361, 2383-2404, 2425-2433, 2445-2455, 2495-2505 and 394-549 of Seq ID No 262; 9-24, 31-53, 57-67, 69-79, 84-114, 133-141, 144-172, 178-186 and 13-46 of Seq ID No 263; 4-25, 27-35, 43-52, 59-70, 79-91, 115-130, 136-152, 154-163, 170-179 and 1-58 of Seq ID No 264; 4-30, 49-55, 71-80, 96-105, 111-126, 139-146, 149-162, 239-245, 279-285, 290-296, 300-307, 331-337, 343-350 and 250-351 of Seq ID No 265; 9-27, 34-41, 43-51, 92-111, 114-120, 123-131, 139-150, 156-171, 176-186, 188-204, 229-241, 252-258, 266-279, 288-297, 319-334, 338-348, 373-379, 389-398, 431-439, 479-484 and 214-398 of Seq ID No 266; 4-15, 18-27, 47-52, 68-83, 91-97, 104-110, 115-121, 139-147, 157-164, 198-206, 227-236, 241-254, 264-273, 278-289, 311-320, 353-361, 372-383, 405-420, 426-434 and 232-386 of Seq ID No 267; 4-10, 24-34, 91-97, 129-141, 156-163, 184-190, 205-219, 229-235, 256-273, 278-285 and 93-116 of Seq ID No 268; 7-29, 35-54, 71-83, 85-91, 104-111, 122-134, 138-144, 146-154, 158-174, 177-183, 186-201, 207-215, 223-235, 240-247, 262-273, 275-283, 287-292 and 48-66 of Seq ID No 269; 7-27, 31-47, 49-70, 75-102, 110-149, 157-171, 217-223, 235-251, 294-302, 358-364, 367-375, 387-393, 395-412, 423-430, 441-451, 456-470, 472-486, 488-495, 499-509, 515-529, 536-549, 556-570, 574-603, 607-615, 625-633, 642-658, 670-676, 683-702, 708-716, 720-726, 747-756, 763-784, 803-812, 815-826 and 475-490 of Seq ID No 270; 7-22, 30-38, 53-59, 64-75, 83-95, 97-112, 120-131, 133-142, 145-151, 154-166, 172-180, 189-203, 227-238, 277-287, 9-156 and 174-287 of Seq ID No 271; 13-23, 25-32, 111-117, 150-164, 185-193, 207-212, 216-224, 230-236, 263-272, 304-311, 342-348, 374-385, 391-407, 444-458, 480-487, 489-499, 523-542, 544-558, 572-579, 620-640, 686-696, 703-710, 742-755, 765-772, 817-822, 830-837, 865-872, 931-937 and 66-86 of Seq ID No 272; 4-27, 49-56, 62-70, 86-92, 121-127, 151-163, 170-182, 195-202, 212-226, 237-243 and 234-254 of Seq ID No 273; 4-10, 13-24, 39-51, 62-78, 92-104, 107-117, 134-141, 156-161, 166-181, 210-216, 222-229, 256-266, 273-280, 297-304, 313-330, 336-349, 371-376, 433-439, 443-448, 488-493, 506-515, 527-534, 560-572, 575-583, 587-593 and 252-483 of Seq ID No 274; 4-15, 21-38, 45-56, 81-95, 102-108, 118-130, 133-147, 152-162, 166-171, 199-204, 211-218, 230-240, 253-261, 274-283, 288-294, 312-317, 325-336, 344-357, 391-414 and 24-146 of Seq ID No 275; 26-31, 38-56, 65-82, 90-101, 112-119, 123-153, 175-188, 197-216, 234-242, 249-265, 273-286, 290-305, 327-335, 338-346, 361-372, 394-404 and 290-306 of Seq ID No 276; 17-26, 43-48, 50-73, 81-93, 95-107, 139-146, 158-168, 171-176, 190-196, 202-212, 216-223, 243-266, 274-282, 308-313, 324-330, 344-378, 380-387, 403-422, 427-443, 448-455, 457-465, 491-515, 517-528, 553-567, 589-599, 610-617, 642-648, 670-697, 709-717, 726-743, 745-759, 769-803, 807-823, 840-849 and 820-851 of Seq ID No 277; 4-18, 39-48, 53-63, 66-90, 102-117, 125-134, 137-145, 156-162, 169-197, 26-40 and 56-80 of Seq ID No 278; 21-33, 36-42, 49-60, 68-76, 91-105, 123-130, 141-161, 169-178, 185-190, 192-199, 205-214, 223-233, 239-247, 260-269, 284-293, 300-314, 324-352, 357-364, 373-382, 389-403, 420-432, 438-446, 466-471, 477-484, 503-509, 549-556, 558-576, 600-623, 625-635, 654-661, 663-669, 671-687, 702-716, 735-741, 744-750, 757-766, 776-786, 807-815, 824-832, 854-860, 863-897, 909-915, 920-946, 952-959, 982-997, 1024-1038, 1049-1055, 1071-1085, 1104-1113, 1121-1132, 1138-1150, 1187-1196, 1212-1221, 1227-1236, 1257-1262, 1264-1278, 1282-1294, 1307-1318, 1353-1370, 1382-1388, 1396-1409, 1434-1440, 1446-1454, 1465-1478, 1485-1513, 1516-1529, 1540-1545, 1563-1568, 1575-1593, 1607-1616, 1628-1645, 1648-1661, 1676-1682, 1689-1697, 1713-1719, 1739-1749, 1753-1758, 1763-1774, 1797-1803, 1807-1846, 1855-1874, 1877-1891, 1893-1907, 1912-1925, 1931-1943, 1955-1965, 1976-1990, 2032-2043, 2045-2051, 2099-2105, 2131-2138, 2161-2179, 2188-2199, 2205-2216, 2219-2227, 2235-2245, 2247-2267, 2277-2288, 2294-2304, 2314-2326, 2346-2358, 2365-2377, 2383-2402, 2407-2423, 2437-2450, 2454-2473, 2489-2497, 2525-2531, 2557-2570, 2580-2587, 2589-2599, 2621-2641, 2647-2653, 2661-2677, 2685-2690, 2697-2717, 2722-2733, 2739-2777, 2786-2793, 2801-2808, 2811-2822, 2825-2835, 2838-2845, 2859-2871, 2877-2883, 213-344, 954-1080 and 2524-2733 of Seq ID No 279; 10-16, 18-23, 28-41, 63-69, 77-91, 101-109, 118-136, 146-153, 155-162, 168-179, 192-207, 217-226, 229-235, 239-254, 279-286, 294-307, 313-319, 334-341, 344-353, 363-377, 390-396 and 178-328 of Seq ID No 280; 18-42, 68-84, 89-95, 100-105, 107-115, 125-135, 154-177, 189-195, 205-228, 236-243, 252-259, 279-300, 309-316, 323-331, 340-351, 353-364, 377-402 and 85-97 of Seq ID No 281; 4-18, 26-32, 66-76, 100-126, 151-159, 178-186, 188-194, 200-210, 241-248, 253-259, 262-279, 284-291, 307-313, 315-322, 327-337, 376-386, 399-407, 432-441, 467-473, 487-497, 499-505, 543-549, 560-568, 585-593, 598-604, 608-614, 630-642, 647-653, 690-703, 717-730, 21-200 and 468-480 of Seq ID No 282; 17-49, 52-58, 62-73, 78-97, 100-117, 122-172, 185-190, 193-217, 225-236 and 33-42 of Seq ID No 283; 7-39, 50-58, 73-89, 96-107, 109-120, 126-142, 152-170, 178-202, 205-211, 224-244, 249-259, 261-270, 300-310, 312-325 and 158-169 of Seq ID No 284; 4-31, 40-64, 71-82, 85-92, 102-124, 126-139, 147-152, 159-173, 176-188, 195-207, 210-216, 234-241, 249-256, 258-276, 279-293, 296-302, 310-315, 349-356, 363-378, 380-403, 411-426, 435-441, 448-459, 463-476, 488-494 and 201-221 of Seq ID No 285; 5-13, 15-74, 87-104, 107-120, 123-129, 136-145, 150-191, 193-206, 227-248, 250-264, 278-302, 304-323, 332-378, 384-407, 409-419, 425-457, 462-471, 474-497, 511-545, 555-564, 571-578, 585-598, 640-647, 669-675, 682-691, 693-705, 729-743, 752-761, 772-780, 786-804, 808-818, 822-846, 858-880, 884-900, 910-939, 941-947, 962-971, 973-988, 998-1003, 1007-1027 and 236-259 of Seq ID No 286; 4-19, 27-68, 81-111, 121-160 and 60-79 of Seq ID No 287; 4-37, 40-46, 52-57, 199-205, 222-229, 236-244, 250-267, 269-282 and 27-197 of Seq ID No 288; 4-16, 24-30, 32-38, 63-75, 86-92, 98-111, 113-126, 160-165, 170-180, 198-204, 227-233, 239-245, 253-273, 308-314, 352-365, 382-387, 395-403, 423-429, 472-482, 484-493, 501-507, 518-526, 536-541, 543-550, 556-562, 586-600, 626-33, 649-661, 680-688 and 546-559 of Seq ID No 289; 16-33, 48-59, 63-71, 77-92, 94-109, 117-124, 139-151, 169-181, 184-227, 233-249, 251-261, 263-275, 282-294, 297-321, 326-332, 341-355, 383-399 and 258-272 of Seq ID No 290; 11-26, 31-39, 43-52, 55-62, 64-70, 80-94, 123-133, 135-141, 172-181, 185-206, 209-218, 224-230, 238-244, 251-262, 264-271, 290-301, 306-324, 333-340, 350-357, 367-375, 390-397, 434-441, 443-448, 77-226 and 350-429 of Seq ID No 291; 4-13, 22-27, 31-45, 50-59, 72-96, 99-114, 131-141, 143-150, 159-176, 180-186, 189-198, 208-214, 234-253, 271-287, 294-299, 310-366, 382-390, 398-416, 424-443 and 283-305 of Seq ID No 292; 9-26, 30-53, 62-72, 86-95, 112-122, 136-145, 153-160, 209-221, 227-237, 241-268, 281-288, 291-298, 308-314, 321-328, 336-346, 351-379, 388-397, 409-416, 423-433, 443-481, 511-519 and 213-232 of Seq ID No 293; 12-18, 25-31, 38-50, 59-67, 71-82, 96-126 and 76-88 of Seq ID No 294; 4-25, 39-44, 64-71, 74-88, 100-113, 128-138, 151-162, 164-177, 185-190, 204-213, 233-239, 246-254, 281-286, 293-306, 309-318, 333-347, 349-359, 385-398, 404-423, 458-465, 477-484, 490-499, 501-533, 554-566, 582-590, 596-616, 624-629, 631-639, 654-680, 694-720, 735-743 and 2-100 of Seq ID No 295; 4-16, 36-41, 52-75, 98-107, 109-117, 122-128, 133-139, 141-155, 159-165, 169-182, 187-193, 195-201, 211-224, 230-236, 247-269, 278-290 and 75-92 of Seq ID No 296; 7-21, 25-33, 37-43, 87-94, 103-120, 131-147, 168-174, 197-203, 207-212, 227-237, 247-257, 263-271, 279-287, 298-306, 320-325, 332-340, 363-374, 379-384, 390-401, 403-414, 428-433, 448-457, 462-475, 483-490, 513-519, 525-535, 543-554, 559-566, 571-620, 625-631, 636-642, 659-670, 688-706, 708-723, 770-779, 787-793, 796-807, 820-840, 848-854, 863-874, 895-905, 912-919, 934-942, 968-975, 983-1000, 1012-1019, 1026-1036, 1050-1060, 1064-1070, 1081-1091, 1094-1108, 1112-1118, 1140-1152, 1164-1169, 1172-1180, 1187-1192 and 732-748 of Seq ID No 297; 23-40, 42-59, 66-73, 78-97, 111-128, 130-141, 157-166, 178-183 and 53-71 of Seq ID No 298; 4-27, 38-44, 47-57, 59-85, 99-106, 114-121, 154-166, 181-186, 193-198, 238-244, 253-262, 272-278, 287-299, 314-320, 338-350, 358-368, 382-388, 407-416, 433-446, 456-461, 463-473 and 86-195 of Seq ID No 299; 5-24, 38-59, 64-80, 87-99, 105-126, 134-142, 149-163, 165-179, 181-202, 205-220, 227-233, 243-250, 257-263 and 87-245 of Seq ID No 300; 5-32, 47-53, 66-79, 81-97, 115-151, 155-174, 183-188, 196-210, 215-226, 230-238, 253-258, 263-270, 276-282, 295-301, 304-325, 334-344, 360-390, 397-412, 425-432, 434-462, 478-494, 508-526, 539-564, 571-579, 347-371 and 375-386 of Seq ID No 301; 4-15, 36-44, 49-56, 60-66, 68-82, 84-103, 109-115, 118-141, 147-154, 160-168, 176-185 and 26-39 of Seq ID No 302; 7-13, 23-33 and 13-21 of Seq ID No 303; 2-10 of Seq ID No 304; 4-9, 12-18, 35-42, 49-62 and 6-18 of Seq ID No 305; 19-25 and 1-13 of Seq ID No 306; 15-21, 27-45 and 12-25 of Seq ID No 307; 14-20 and 1-14 of Seq ID No 308; 4-18 and 13-26 of Seq ID No 309; 8-21 and 2-20 of Seq ID No 310; 4-14 and 4-16 of Seq ID No 311; 3-12 of Seq ID No 312; 6-14, 6-25, 35-57 and 2-14 of Seq ID No 313; 6-25, 35-57 and 17-31 of Seq ID No 314; 14-25, 32-46 and 5-19 of Seq ID No 315; 18-31 and 5-16 of Seq ID No 316; 19-24 and 4-26 of Seq ID No 317; 13-21, 29-34, 47-58, 61-73 and 36-47 of Seq ID No 318; 4-15 and 5-24 of Seq ID No 319; 6-18 of Seq ID No 320; 13-20 and 4-13 of Seq ID No 321; 15-23 of Seq ID No 322; 4-9 and 7-21 of Seq ID No 323; 1-10 of Seq ID No 324; 4-14 of Seq ID No 325; 4-17, 35-41, 46-89, 93-98 and 70-88 of Seq ID No 326; 1-13 of Seq ID No 327; 4-16, 26-32 and 25-38 of Seq ID No 328; 8-15, 23-28 and 4-17 of Seq ID No 329; 4-12 and 1-15 of Seq ID No 330; 4-29, 31-42, 52-58 and 6-16 of Seq ID No 331; 4-9, 24-32 and 9-19 of Seq ID No 332; 4-12, 18-27 and 5-18 of Seq ID No 333; 4-11, 37-56, 58-92 and 18-29 of Seq ID No 334; 8-28 and 20-35 of Seq ID No 335; 4-15 of Seq ID No 336; 4-23, 27-39, 55-63 and 35-58 of Seq ID No 337; 6-26, 28-54 and 28-47 of Seq ID No 338; 4-10, 38-52, 58-82 and 30-49 of Seq ID No 339; 4-22, 29-35, 44-50, 53-68, 70-80 and 20-33 of Seq ID No 340; 22-28, 30-36 and 18-33 of Seq ID No 341; 4-11, 13-21, 25-30 and 20-30 of Seq ID No 342; 10-22 and 10-23 of Seq ID No 343; 4-11 and 9-20 of Seq ID No 344; 14-25, 32-46 and 6-19 of Seq ID No 345; 5-30 and 14-33 of Seq ID No 346; 4-15, 28-35, 46-55, 59-65, 76-84 and 9-24 of Seq ID No 347; 27-33 and 5-19 of Seq ID No 348; 5-13 and 8-18 of Seq ID No 349; 9-22, 24-34 and 21-40 of Seq ID No 350; 4-17, 35-41, 46-89, 93-98 and 71-89 of Seq ID No 351; 4-12, 14-24 and 2-17 of Seq ID No 352; 9-17 and 5-16 of Seq ID No 353; 7-41, 48-58, 63-75, 80-89 and 43-53 of Seq ID No 354; 4-22, 25-30 and 4-14 of Seq ID No 355; 4-55 and 18-33 of Seq ID No 356; 262-280 of Seq ID No 179; 131-146 of Seq ID No 186; 207-224 of Seq ID No 188; 27-50, 203-217 and 313-325 of Seq ID No 189; 110-129 of Seq ID No 192; 156-179, 174-197, 192-215, 210-233, 228-251 and 246-267 of Seq ID No 190; 377-400 of Seq ID No 196; 34-43, 234-257 and 350-367 of Seq ID No 203; 304-327 of Seq ID No 207; 25-48, 43-66 and 61-82 of Seq ID No 222; 398-421, 416-439, 434-457, 452-475, 470-493, 488-511, 506-529, 524-547, 621-644, 639-664, 707-730, 725-748, 743-766, 761-784, 779-802, 797-820, 984-1007, 1002-1025, 1020-1043, 1038-1061, 1056-1079, 1074-1097, 1092-1115, 1286-1309, 1304-1327, 1322-1345, 1340-1363, 1358-1381, 1376-1399, 1394-1417, 1412-1435, 1430-1453, 1448-1471, 1466-1489 and 1484-1507 of Seq ID No 226; 188-211, 206-229, 224-247, 242-265, 260-283 and 278-296 of Seq ID No 227; 56-79 and 122-132 of Seq ID No 229; 35-46 of Seq ID No 231; 178-201, 196-219, 214-237, 232-255, 250-273, 268-291, 379-402, 397-420, 415-438, 433-456, 451-474, 642-65, 660-683, 678-701, 696-719, 714-737, 732-755, 750-773, 768-791, 899-922, 917-940, 935-958, 1037-1060, 1055-1078, 1073-1096 and 1091-1114 of Seq ID No 232; 330-346 of Seq ID No 233; 571-594, 589-612, 607-630, 625-648, 643-666 and 661-684 of Seq ID No 242; 188-207 of Seq ID No 244; 61-84, 308-331, 326-349, 344-367, 362-385, 380-403 and 398-421 of Seq ID No 249; 79-98, 345-366, 844-867, 870-887 and 890-905 of Seq ID No 258; 94-109 of Seq ID No 268; 188-207 of Seq ID No 272; 290-306 of Seq ID No 276; 826-849 of Seq ID No 277; 228-252, 247-270, 265-288, 283-306, 301-324, 955-978, 973-996, 991-1014, 1009-1032, 1027-1050, 1045-1068, 2533-2556, 2551-2574, 2569-2592, 2587-2610, 2605-2628 and 2623-2646 of Seq ID No 279; 86-109 and 104-127 of Seq ID No 288; 546-560 of Seq ID No 289; 260-271 of Seq ID No 290; 106-129, 124-147, 142-165, 160-183, 178-201 and 375-398 of Seq ID No 291; 284-307 of Seq ID No 292; 362-385 of Seq ID No 301.

The present invention also provides a process for producing a H. pylori hyperimmune serum reactive antigen or a fragment thereof according to the present invention comprising expressing one or more of the nucleic acid molecules according to the present invention in a suitable expression system.

Moreover, the present invention provides a process for producing a cell, which expresses a H. pylori hyperimmune serum reactive antigen or a fragment thereof according to the present invention comprising transforming or transfecting a suitable host cell with the vector according to the present invention.

According to the present invention a pharmaceutical composition, especially a vaccine, comprising a hyperimmune serum-reactive antigen or a fragment thereof as defined in the present invention or a nucleic acid molecule as defined in the present invention is provided.

In a preferred embodiment the pharmaceutical composition further comprises an immunostimulatory substance, preferably selected from the group comprising polycationic polymers, especially polycationic peptides, immunostimulatory deoxynucleotides (ODNs), peptides containing at least two LysLeuLys motifs, especially KLKL5KLK, neuroactive compounds, especially human growth hormone, alumn, Freund's complete or incomplete adjuvants or combinations thereof.

In a more preferred embodiment the immunostimulatory substance is a combination of either a polycationic polymer and immunostimulatory deoxynucleotides or of a peptide containing at least two LysLeuLys motifs and immunostimulatory deoxynucleotides.

In a still more preferred embodiment the polycationic polymer is a polycationic peptide, especially polyarginine.

According to the present invention the use of a nucleic acid molecule according to the present invention or a hyperimmune serum-reactive antigen or fragment thereof according to the present invention for the manufacture of a pharmaceutical preparation, especially for the manufacture of a vaccine against H. pylori infection, is provided.

Also an antibody, or at least an effective part thereof, which binds at least to a selective part of the hyperimmune serum-reactive antigen or a fragment thereof according to the present invention is provided herewith.

In a preferred embodiment the antibody is a monoclonal antibody.

In another preferred embodiment the effective part of the antibody comprises Fab fragments.

In a further preferred embodiment the antibody is a chimeric antibody.

In a still preferred embodiment the antibody is a humanized antibody.

The present invention also provides a hybridoma cell line, which produces an antibody according to the present invention.

Moreover, the present invention provids a method for producing an antibody according to the present invention, characterized by the following steps:
initiating an immune response in a non-human animal by administrating an hyperimmune serum-reactive antigen or a fragment thereof, as defined in the invention, to said animal,
removing an antibody containing body fluid from said animal, and
producing the antibody by subjecting said antibody containing body fluid to further purification steps.

Accordingly, the present invention also provides a method for producing an antibody according to the present invention, characterized by the following steps:
a initiating an immune response in a non-human animal by administrating an hyperimmune serum-reactive antigen or a fragment thereof, as defined in the present invention, to said animal,
removing the spleen or spleen cells from said animal,
producing hybridoma cells of said spleen or spleen cells,
selecting and cloning hybridoma cells specific for said hyperimmune serum-reactive antigens or a fragment thereof,
producing the antibody by cultivation of said cloned hybridoma cells and optionally further purification steps.

The antibodies provided or produced according to the above methods may be used for the preparation of a medicament for treating or preventing H. pylori infections.

According to another aspect the present invention provides an antagonist, which binds to a hyperimmune serum-reactive antigen or a fragment thereof according to the present invention.

Such an antagonist capable of binding to a hyperimmune serum-reactive antigen or fragment thereof according to the present invention may be identified by a method comprising the following steps:
a) contacting an isolated or immobilized hyperimmune serum-reactive antigen or a fragment thereof according to the present invention with a candidate antagonist under conditions to permit binding of said candidate antagonist to said hyperimmune serum-reactive antigen or fragment, in the presence of a component capable of providing a detectable signal in response to the binding of the candidate antagonist to said hyperimmune serum reactive antigen or fragment thereof; and
b) detecting the presence or absence of a signal generated in response to the binding of the antagonist to the hyperimmune serum reactive antigen or the fragment thereof.

An antagonist capable of reducing or inhibiting the interaction activity of a hyperimmune serum-reactive antigen or a fragment thereof according to the present invention to its interaction partner may be identified by a method comprising the following steps:
a) providing a hyperimmune serum reactive antigen or a hyperimmune fragment thereof according to the present invention,
b) providing an interaction partner to said hyperimmune serum reactive antigen or a fragment thereof, especially an antibody according to the present invention,
c) allowing interaction of said hyperimmune serum reactive antigen or fragment thereof to said interaction partner to form an interaction complex,
d) providing a candidate antagonist,
e) allowing a competition reaction to occur between the candidate antagonist and the interaction complex,
f) determining whether the candidate antagonist inhibits or reduces the interaction activities of the hyperimmune serum reactive antigen or the fragment thereof with the interaction partner.

The hyperimmune serum reactive antigens or fragments thereof according to the present invention may be used for the isolation and/or purification and/or identification of an interaction partner of said hyperimmune serum reactive antigen or fragment thereof.

The present invention also provides a process for in vitro diagnosing a disease related to expression of a hyperimmune serum-reactive antigen or a fragment thereof according to the present invention comprising determining the presence of a nucleic acid sequence encoding said hyperimmune serum reactive antigen or fragment thereof according to the present invention or the presence of the hyperimmune serum reactive antigen or fragment thereof according to the present invention.

The present invention also provides a process for in vitro diagnosis of a bacterial infection, especially a H. pylori infection, comprising analyzing for the presence of a nucleic acid sequence encoding said hyperimmune serum reactive antigen or fragment thereof according to the present invention or the presence of the hyperimmune serum reactive antigen or fragment thereof according to the present invention.

Moreover, the present invention provides the use of a hyperimmune serum reactive antigen or fragment thereof according to the present invention for the generation of a peptide binding to said hyperimmune serum reactive antigen or fragment thereof, wherein the peptide is an anticaline.

The present invention also provides the use of a hyperimmune serum-reactive antigen or fragment thereof according to the present invention for the manufacture of a functional nucleic acid, wherein the functional nucleic acid is selected from the group comprising aptamers and spiegelmers.

The nucleic acid molecule according to the present invention may also be used for the manufacture of a functional ribonucleic acid, wherein the functional ribonucleic acid is selected from the group comprising ribozymes, antisense nucleic acids and siRNA.

The present invention advantageously provides an efficient, relevant and comprehensive set of isolated nucleic acid molecules and their encoded hyperimmune serum reactive antigens or fragments thereof identified from H. pylori using an antibody preparation from multiple human plasma pools and surface expression libraries derived from the genome of H. pylori. Thus, the present invention fulfils a widely felt demand for H. pylori antigens, vaccines, diagnostics and products useful in procedures for preparing antibodies and for identifying compounds effective against H. pylori infection.

An effective vaccine should be composed of proteins or polypeptides, which are expressed by all strains and are able to induce high affinity, abundant antibodies against cell surface components of H. pylori. The antibodies should be IgG1 and/or IgG3 for opsonization, and any IgG subtype and IgA for neutralisation of adherence and toxin action. A chemically defined vaccine must be definitely superior compared to a whole cell vaccine (attenuated or killed), since components of H. pylori, which cross-react with human tissues or inhibit opsonization can be eliminated, and the individual proteins inducing protective antibodies and/or a protective immune response can be selected.

The approach, which has been employed for the present invention, is based on the interaction of H. pylori proteins or peptides with the antibodies present in human sera. The antibodies produced against H. pylori by the human immune system and present in human sera are indicative of the in vivo expression of the antigenic proteins and their immunogenicity. In addition, the antigenic proteins as identified by the bacterial surface display expression libraries using pools of pre-selected sera are processed in a second and third round of screening by individual selected or generated sera. Thus the present invention supplies an efficient, relevant, comprehensive set of H. pylori antigens as promising candidates for the development of a pharmaceutical composition, especially a vaccine preventing infection by H. pylori.

In the antigen identification program for identifying a comprehensive set of antigens according to the present invention, at least two different bacterial surface expression libraries are screened with several serum pools or plasma fractions or other pooled antibody containing body fluids (antibody pools). The antibody pools are derived from a serum collection, which has been tested against antigenic compounds of H. pylori, such as whole cell extracts and culture supernatant proteins. Preferably, 2 distinct serum collections are used: 1. With very stable antibody repertoire: normal adults, clinically healthy people, who are non-carriers and overcame previous encounters or currently carriers of H. pylori without acute disease and symptoms, 2. With antibodies induced acutely by the presence of the pathogenic organism: patients with manifest disease (e.g. H. pylori gastritis, peptic ulcer disease or gastric cancer). Sera have to react with multiple H. pylori-specific antigens in order to be considered hyperimmune and therefore relevant in the screening method applied for the present invention. The antibodies produced against H. pylori by the human immune system and present in human sera are indicative of the in vivo expression of the antigenic proteins and their immunogenicity.

The expression libraries as used in the present invention should allow expression of all potential antigens, e.g. derived from all surface proteins of H. pylori. Bacterial surface display libraries will be represented by a recombinant library of a bacterial host displaying a (total) set of expressed peptide sequences of H. pylori on a number of selected outer membrane proteins (LamB, FhuA) at the bacterial host membrane {Georgiou, G., 1997}; {Etz, H. et al., 2001}. One of the advantages of using recombinant expression libraries is that the identified hyperimmune serum-reactive antigens may be instantly produced by expression of the coding sequences of the screened and selected clones expressing the hyperimmune serum-reactive antigens without further recombinant DNA technology or cloning steps necessary.

The comprehensive set of antigens identified by the described program according to the present invention is analysed further by additional rounds of screening. Therefore individual antibody preparations or antibodies generated against selected peptides, which were identified as immunogenic are used. According to a preferred embodiment the individual antibody preparations for the second round of screening are derived from patients who have suffered from infection with H. pylori, especially from patients who show an antibody titer above a certain minimum level, for example an antibody titer being higher than 80 percentile, preferably higher than 90 percentile, especially higher than 95 percentile of the human (patient or healthy individual) sera tested. Using such high titer individual antibody preparations in the second screening round allows a very selective identification of the hyperimmune serum-reactive antigens and fragments thereof from H. pylori.

Following the high throughput screening procedure, the selected antigenic proteins, expressed as recombinant proteins or in vitro translated products, in case it can not be expressed in prokaryotic expression systems, or the identified antigenic peptides (produced synthetically) are tested in a second screening by a series of ELISA and Western blotting assays for the assessment of their immunogenicity with a large human serum collection (>50 uninfected, >100 patients sera).

It is important that the individual antibody preparations (which may also be the selected serum) allow a selective identification of the hyperimmune serum-reactive antigens from all the promising candidates from the first round. Therefore, preferably at least 10 individual antibody preparations (i.e. antibody preparations (e.g. sera) from at least 10 different individuals having suffered from an infection to the chosen pathogen) should be used in identifying these antigens in the second screening round. It is possible to use also less than 10 individual preparations, however, selectivity of the step may not be optimal with a low number of individual antibody preparations. Therefore, recognition of a given hyperimmune serum-reactive antigen (or an antigenic fragment thereof) by at least 10 individual antibody preparations, preferably at least 30, especially at least 50 individual antibody preparations confers proper selectivity in the identification process. Hyperimmune serum-reactivity may of course be tested with as many individual preparations as possible (e.g. with more than 100 or even with more than 1,000).

Therefore, the relevant portion of the hyperimmune serum-reactive antibody preparations according to the method of the present invention should preferably be at least 10, more preferred at least 30, especially at least 50 individual antibody preparations. Alternatively (or in combination) hyperimmune serum-reactive antigens may preferably be also identified with at least 20%, preferably at least 30%, especially at least 40% of all individual antibody preparations used in the second screening round.

According to a preferred embodiment of the present invention, the sera from which the individual antibody preparations for the second round of screening are prepared (or which are used as antibody preparations), are selected by their titer against H. pylori (e.g. against a preparation of this pathogen, such as a lysate, cell wall components and recombinant proteins). Preferably, some are selected with a total IgA titer above 4,000 U, especially above 6,000 U, and/or an IgG titer above 10,000 U, especially above 12,000 U (U=units, calculated from the OD₄₀₅ₙₘ reading at a given dilution) when the whole organism (total lysate or whole cells) is used as antigen in the ELISA.

The antibodies produced against Helicobacter by the human immune system and present in human sera are indicative of the in vivo expression of the antigenic proteins and their immunogenicity. The recognition of linear epitopes by antibodies can be based on sequences as short as 45 amino acids. It, however, does not necessarily mean that these short peptides are capable of inducing the given antibody in vivo. For that reason the defined epitopes, polypeptides and proteins are further to be tested in animals (mainly in mice) for their capacity to induce antibodies against the selected proteins in vivo.

The preferred antigens are located on the cell surface or secreted, and are therefore accessible extracellularly. Antibodies against cell wall proteins are expected to serve two purposes: to inhibit adhesion and to promote phagocytosis or complement mediated killing. Antibodies against secreted proteins are beneficial in neutralisation of their function as toxin or virulence component. It is also known that bacteria communicate with each other through secreted proteins. Neutralizing antibodies against these proteins will interrupt growth-promoting cross-talk between or within Helicobacter species. Bioinformatic analyses (signal sequences, cell wall localisation signals, transmembrane domains) proved to be very useful in assessing cell surface localisation or secretion. The experimental approach includes the isolation of antibodies with the corresponding epitopes and proteins from human serum, and the generation of immune sera in mice against (poly) peptides selected by the bacterial surface display screens. These sera are then used in a third round of screening as reagents in the following assays: cell surface staining of Helicobacter grown under different conditions (FACS, microscopy), determination of neutralizing capacity (toxin, adherence), and promotion of opsonization and phagocytosis (in vitro phagocytosis assay).

For that purpose, bacterial E. coli clones are directly injected into mice and immune sera taken and tested in the relevant in vitro assay for functional opsonic or neutralizing antibodies. Alternatively, specific antibodies may be purified from human or mouse sera using peptides or proteins as substrate.

It is not clear as to what extend host defence against H. pylori relies on innate or adaptive immunological mechanisms. The mucous membranes and the gastric acidic environment are formidable barriers against invasion by Helicobacter. However, once the mucous membranes are breached the first line of non-adaptive cellular defence begins its co-ordinate action through complement and phagocytes, especially the polymorphonuclear leukocytes (PMNs) as indicated by the massive neutrophil infiltration of the gastric mucosa in response to the presence of H. pylori. Attachment of H. pylori induces strong pro-inflammatory cytokine release, including TNF-α, IL-1β and IL-8 that can mediate a local chemoattractant effect for immuno-effector cells, such as granulocytes {Prinz, C. et al., 2003}; {Sutton, P., 2001}. These cells can be regarded as the cornerstones in eliminating invading bacteria. As H. pylori is thought to be a exclusively extracellular pathogen, the major anti-Helicobacter adaptive response should come from the humoral arm of the immune system, and this seems to be in agreement with the high titers of primarily IgG and IgA antibodies that develop in patient upon H. pylori infection {Prinz, C. et al., 2003}; {Sutton, P., 2001}. The induction of high titer IgG and sectretory IgA type antibody response may reflect the importance of adaptive mechanisms in the immune response against this organism. In principle, the effect of these antibodies is mediated through three major mechanisms: promotion of opsonization, toxin neutralization, and inhibition of adherence. It is believed that opsonization is especially important, because of its requirement for an effective phagocytosis. For efficient opsonization the microbial surface has to be coated with antibodies and complement factors for recognition by PMNs through receptors for the Fc fragment of IgG molecules or for activated C3b. After opsonization, the bacteria are phagocytosed and killed. Antibodies bound to specific antigens on the cell surface of bacteria serve as ligands for the attachment to PMNs and to promote phagocytosis. The very same antibodies bound to the adhesins and other cell surface proteins are expected to neutralize adhesion and prevent colonization.

Inducing high affinity antibodies of the opsonic and neutralizing type by vaccination helps the innate immune system to eliminate bacteria and toxins. This makes the method according to the present invention an optimal tool for the identification of H. pylori antigenic proteins. The selection of antigens as provided by the present invention is thus well suited to identify those that will lead to protection against infection in an animal model or in humans.

However, there is compelling evidence indicating that antibodies are not required for immunisation-induced effective immunity against gastric helicobacters. Indeed, in vitro studies have demonstrated that diminutive fraction of the colonizing H. pylori population might enter epithelial cells and this is in good agreement with the fact that H. pylori generally induces a predominantly T helper 1 (Th1) type immune response, normally associated with invasive bacteria. Gastric T cells isolated from infected animals and humans produce TNF-α and IFN-γ but not IL-4, typical for a Th1-biased response. This pro-inflammatory Th1 response is clearly not effective against infection {Prinz, C. et al., 2003}; {Sutton, P., 2001}. Current vaccination protocols, such as immunization with recombinant UreB, can drive the immune response to a polarized Th2 phenotype. Studies with knockout mice demonstrated that immunization with urease is possible when the Th2 response is absent. Therefore, vaccine development using new antigens as well as suitable adjuvants that are capable of inducing strong Th1-biased responses may be beneficial in disease protection caused by H. pylori.

According to the antigen identification method used herein, the present invention can surprisingly provide a set of comprehensive novel nucleic acids and novel hyperimmune serum reactive antigens and fragments thereof of H. pylori, among other things, as described below. According to one aspect, the invention particularly relates to the nucleotide sequences encoding hyperimmune serum reactive antigens which sequences are set forth in the Sequence listing Seq ID No 1-178, and the corresponding encoded amino acid sequences representing hyperimmune serum reactive antigens are set forth in the Sequence Listing Seq ID No 179-356.

In a preferred embodiment of the present invention, a nucleic acid molecule is provided which exhibits 70% identity over their entire length to a nucleotide sequence set forth with Seq ID No 3-4, 16, 19-21, 28-29, 33-38, 41-42, 44, 48-52, 55, 57-58, 61, 63, 65, 67-68, 72, 74-75, 81, 84, 91, 94, 96-97, 101, 105-108, 112, 115-117, 119, 123-178. Most highly preferred are nucleic acids that comprise a region that is at least 80% or at least 85% identical over their entire length to a nucleic acid molecule set forth with Seq ID No 3-4, 16, 19-21, 28-29, 33-38, 41-42, 44, 48-52, 55, 57-58, 61, 63, 65, 67-68, 72, 74-75, 81, 84, 91, 94, 96-97, 101, 105-108, 112, 115-117, 119, 123-178. In this regard, nucleic acid molecules at least 90%, 91%, 92%, 93%, 94%, 95%, or 96% identical over their entire length to the same are particularly preferred. Furthermore, those with at least 97% are highly preferred, those with at least 98% and at least 99% are particularly highly preferred, with at least 99% or 99.5% being the more preferred, with 100% identity being especially preferred. Moreover, preferred embodiments in this respect are nucleic acids which encode hyperimmune serum reactive antigens or fragments thereof (polypeptides) which retain substantially the same biological function or activity as the mature polypeptide encoded by said nucleic acids set forth in the Seq ID No 3-4, 16, 19-21, 28-29, 33-38, 41-42, 44, 48-52, 55, 57-58, 61, 63, 65, 67-68, 72, 74-75, 81, 84, 91, 94, 96-97, 101, 105-108, 112, 115-117, 119, 123-178.

Identity, as known in the art and used herein, is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity can be readily calculated. While there exist a number of methods to measure identity between two polynucleotides or two polypeptide sequences, the term is well known to skilled artisans (e.g. Sequence Analysis in Molecular Biology, von Heinje, G., Academic Press, 1987). Preferred methods to determine identity are designed to give the largest match between the sequences tested. Methods to determine identity are codified in computer programs. Preferred computer program methods to determine identity between two sequences include, but are not limited to, GCG program package {Devereux, J. et al., 1984}, BLASTP, BLASTN, and FASTA {Altschul, S. et al., 1990}.

According to another aspect of the invention, nucleic acid molecules are provided which exhibit at least 96% identity to the nucleic acid sequence set forth with Seq ID No 8-10, 13-15, 17-18, 24, 27, 32, 39-40, 45-47, 56, 59, 62, 69-70, 73, 77, 79, 82, 85-86, 88, 90, 103, 109-110, 114, 121.

According to a further aspect of the present invention, nucleic acid molecules are provided which are identical to the nucleic acid sequences set forth with Seq ID No 5, 7, 30-31, 53, 60, 66, 76, 83, 87, 92, 99, 120.

The nucleic acid molecules according to the present invention can as a second alternative also be a nucleic acid molecule which is at least essentially complementary to the nucleic acid described as the first alternative above. As used herein complementary means that a nucleic acid strand is base pairing via Watson-Crick base pairing with a second nucleic acid strand. Essentially complementary as used herein means that the base pairing is not occurring for all of the bases of the respective strands but leaves a certain number or percentage of the bases unpaired or wrongly paired. The percentage of correctly pairing bases is preferably at least 70%, more preferably 80%, even more preferably 90% and most preferably any percentage higher than 90%. It is to be noted that a percentage of 70% matching bases is considered as homology and the hybridization having this extent of matching base pairs is considered as stringent. Hybridization conditions for this kind of stringent hybridization may be taken from Current Protocols in Molecular Biology (John Wiley and Sons, Inc, 1987). More particularly, the hybridization conditions can be as follows:
Hybridization performed e.g. in 5×SSPE, 5×Denhardt's reagent, 0.1% SDS, 100 g/mL sheared DNA at 68° C.
Moderate stringency wash in 0.2×SSC, 0.1% SDS at 42° C.
High stringency wash in 0.1×SSC, 0.1% SDS at 68° C.

Genomic DNA with a GC content of 50% has an approximate TM of 96° C. For 1% mismatch, the TM is reduced by approximately 1° C.

In addition, any of the further hybridization conditions described herein is in principle applicable as well.

All nucleic acid sequence molecules which encode the same polypeptide molecule as those identified by the present invention are encompassed by any disclosure of a given coding sequence, since the degeneracy of the genetic code is directly applicable to unambiguously determine all possible nucleic acid molecules which encode a given polypeptide molecule, even if the number of such degenerated nucleic acid molecules may be high. This is also applicable for fragments of a given polypeptide, as long as the fragments encode a polypeptide being suitable to be used in a vaccination connection, e.g. as an active or passive vaccine.

The nucleic acid molecule according to the present invention can as a third alternative also be a nucleic acid which comprises a stretch of at least 15 bases of the nucleic acid molecule according to the first and second alternative of the nucleic acid molecules according to the present invention as outlined above. Preferably, the bases form a contiguous stretch of bases. However, it is also within the scope of the present invention that the stretch consists of two or more moieties, which are separated by a number of bases.

The present nucleic acids may preferably consist of at least 20, even more preferred at least 30, especially at least 50 contiguous bases from the sequences disclosed herein. The suitable length may easily be optimized due to the planned area of use (e.g. as (PCR) primers, probes, capture molecules (e.g. on a (DNA) chip), etc.). Preferred nucleic acid molecules contain at least a contiguous 15 base portion of one or more of the predicted immunogenic amino acid sequences listed in tables 1 and 2, especially the sequences of table 2 with scores of more than 10, preferably more than 20, especially with a score of more than 25. Specifically preferred are nucleic acids containing a contiguous portion of a DNA sequence of any sequence in the sequence protocol of the present application which shows 1 or more, preferably more than 2, especially more than 5, non-identical nucleic acid residues compared to the published Helicobacter pylori strain 26695 and J99 genomes (Nature, 388: 539-547 (1997), 4658-4663; GenBank accession AE000511 and Nature, 397: 176-180 (1999), GenBank accession AE001439) and/or any other published H. pylori genome sequence or parts thereof. Specifically preferred non-identical nucleic acid residues are residues, which lead to a non-identical amino acid residue. Preferably, the nucleic acid sequences encode for polypeptides having at least 1, preferably at least 2, preferably at least three different amino acid residues compared to the published H. pylori counterparts mentioned above. Also such isolated polypeptides, being fragments of the proteins (or the whole protein) mentioned herein e.g. in the sequence listing, having at least 6, 7, or 8 amino acid residues and being encoded by these nucleic acids are preferred.

The nucleic acid molecule according to the present invention can as a fourth alternative also be a nucleic acid molecule which anneals under stringent hybridisation conditions to any of the nucleic acids of the present invention according to the above outlined first, second, and third alternative. Stringent hybridisation conditions are typically those described herein.

Finally, the nucleic acid molecule according to the present invention can as a fifth alternative also be a nucleic acid molecule which, but for the degeneracy of the genetic code, would hybridise to any of the nucleic acid molecules according to any nucleic acid molecule of the present invention according to the first, second, third, and fourth alternative as outlined above. This kind of nucleic acid molecule refers to the fact that preferably the nucleic acids according to the present invention code for the hyperimmune serum reactive antigens or fragments thereof according to the present invention. This kind of nucleic acid molecule is particularly useful in the detection of a nucleic acid molecule according to the present invention and thus the diagnosis of the respective microorganisms such as H. pylori and any disease or diseased condition where this kind of microorganim is involved. Preferably, the hybridisation would occur or be preformed under stringent conditions as described in connection with the fourth alternative described above.

Nucleic acid molecule as used herein generally refers to any ribonucleic acid molecule or deoxyribonucleic acid molecule, which may be unmodified RNA or DNA or modified RNA or DNA. Thus, for instance, nucleic acid molecule as used herein refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term nucleic acid molecule includes DNAs or RNAs as described above that contain one or more modified bases. Thus, DNAs or RNAs with backbones modified for stability or for other reasons are “nucleic acid molecule” as that term is intended herein. Moreover, DNAs or RNAs comprising unusual bases, such as inosine, or modified bases, such as tritylated bases, to name just two examples, are nucleic acid molecule as the term is used herein. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia. The term nucleic acid molecule also embraces short nucleic acid molecules often referred to as oligonucleotide(s). “Polynucleotide” and “nucleic acid” or “nucleic acid molecule” are often used interchangeably herein.

Nucleic acid molecules provided in the present invention also encompass numerous unique fragments, both longer and shorter than the nucleic acid molecule sequences set forth in the sequencing listing of the H. pylori coding regions, which can be generated by standard cloning methods. To be unique, a fragment must be of sufficient size to distinguish it from other known nucleic acid sequences, most readily determined by comparing any selected H. pylori fragment to the nucleotide sequences in computer databases such as GenBank.

Additionally, modifications can be made to the nucleic acid molecules and polypeptides that are encompassed by the present invention. For example, nucleotide substitutions can be made which do not affect the polypeptide encoded by the nucleic acid, and thus any nucleic acid molecule which encodes a hyperimmune serum reactive antigen or fragments thereof is encompassed by the present invention.

Furthermore, any of the nucleic acid molecules encoding hyperimmune serum reactive antigens or fragments thereof provided by the present invention can be functionally linked, using standard techniques such as standard cloning techniques, to any desired regulatory sequences, whether a H. pylori regulatory sequence or a heterologous regulatory sequence, heterologous leader sequence, heterologous marker sequence or a heterologous coding sequence to create a fusion protein.

Nucleic acid molecules of the present invention may be in the form of RNA, such as mRNA or cRNA, or in the form of DNA, including, for instance, cDNA and genomic DNA obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand.

The present invention further relates to variants of the herein above described nucleic acid molecules which encode fragments, analogs and derivatives of the hyperimmune serum reactive antigens and fragments thereof having a deducted H. pylori amino acid sequence set forth in the Sequence Listing. A variant of the nucleic acid molecule may be a naturally occurring variant such as a naturally occurring allelic variant, or it may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions. Preferred are nucleic acid molecules encoding a variant, analog, derivative or fragment, or a variant, analogue or derivative of a fragment, which have a H. pylori sequence as set forth in the Sequence Listing, in which several, a few, 5 to 10, 1 to 5, 1 to 3, 2, 1 or no amino acid(s) is substituted, deleted or added, in any combination. Especially preferred among these are silent substitutions, additions and deletions, which do not alter the properties and activities of the H. pylori polypeptides set forth in the Sequence Listing. Also especially preferred in this regard are conservative substitutions.

The peptides and fragments according to the present invention also include modified epitopes wherein preferably one or two of the amino acids of a given epitope are modified or replaced according to the rules disclosed in e.g. {Tourdot, S. et al., 2000}, as well as the nucleic acid sequences encoding such modified epitopes.

It is clear that also epitopes derived from the present epitopes by amino acid exchanges improving, conserving or at least not significantly impeding the T cell activating capability of the epitopes are covered by the epitopes according to the present invention. Therefore the present epitopes also cover epitopes, which do not contain the original sequence as derived from H. pylori, but trigger the same or preferably an improved T cell response. These epitope are referred to as “heteroclitic”; they need to have a similar or preferably greater affinity to MHC/HLA molecules, and the need the ability to stimulate the T cell receptors (TCR) directed to the original epitope in a similar or preferably stronger manner.

Heteroclitic epitopes can be obtained by rational design i.e. taking into account the contribution of individual residues to binding to MHC/HLA as for instance described by {Rammensee, H. et al., 1999}, combined with a systematic exchange of residues potentially interacting with the TCR and testing the resulting sequences with T cells directed against the original epitope. Such a design is possible for a skilled man in the art without much experimentation.

Another possibility includes the screening of peptide libraries with T cells directed against the original epitope. A preferred way is the positional scanning of synthetic peptide libraries. Such approaches have been described in detail for instance by {Hemmer, B. et al., 1999} and the references given therein.

As an alternative to epitopes represented by the present derived amino acid sequences or heteroclitic epitopes, also substances mimicking these epitopes e.g. “peptidemimetica” or “retro-inverso-peptides” can be applied.

Another aspect of the design of improved epitopes is their formulation or modification with substances increasing their capacity to stimulate T cells. These include T helper cell epitopes, lipids or liposomes or preferred modifications as described in WO 01/78767.

Another way to increase the T cell stimulating capacity of epitopes is their formulation with immune stimulating substances for instance cytokines or chemokines like interleukin-2, -7, -12, -18, class I and II interferons (IFN), especially IFN-gamma, GM-CSF, TNF-alpha, flt3-ligand and others.

As discussed additionally herein regarding nucleic acid molecule assays of the invention, for instance, nucleic acid molecules of the invention as discussed above, may be used as a hybridization probe for RNA, cDNA and genomic DNA to isolate full-length cDNAs and genomic clones encoding polypeptides of the present invention and to isolate cDNA and genomic clones of other genes that have a high sequence similarity to the nucleic acid molecules of the present invention. Such probes generally will comprise at least 15 bases. Preferably, such probes will have at least 20, at least 25 or at least 30 bases, and may have at least 50 bases. Particularly preferred probes will have at least 30 bases, and will have 50 bases or less, such as 30, 35, 40, 45, or 50 bases.

For example, the coding region of a nucleic acid molecule of the present invention may be isolated by screening a relevant library using the known DNA sequence to synthesize an oligonucleotide probe. A labeled oligonucleotide having a sequence complementary to that of a gene of the present invention is then used to screen a library of cDNA, genomic DNA or mRNA to determine to which members of the library the probe hybridizes.

The nucleic acid molecules and polypeptides of the present invention may be employed as reagents and materials for development of treatments of and diagnostics for disease, particularly human disease, as further discussed herein relating to nucleic acid molecule assays, inter alia.

The nucleic acid molecules of the present invention that are oligonucleotides can be used in the processes herein as described, but preferably for PCR, to determine whether or not the H. pylori genes identified herein in whole or in part are present and/or transcribed in infected tissue such as blood. It is recognized that such sequences will also have utility in diagnosis of the stage of infection and type of infection the pathogen has attained. For this and other purposes the arrays comprising at least one of the nucleic acids according to the present invention as described herein, may be used.

The nucleic acid molecules according to the present invention may be used for the detection of nucleic acid molecules and organisms or samples containing these nucleic acids. Preferably such detection is for diagnosis, more preferable for the diagnosis of a disease related or linked to the present or abundance of H. pylori.

Eukaryotes (herein also “individual(s)”), particularly mammals, and especially humans, infected with H. pylori may be identifiable by detecting any of the nucleic acid molecules according to the present invention detected at the DNA level by a variety of techniques. Preferred nucleic acid molecules candidates for distinguishing a H. pylori from other organisms can be obtained.

The invention provides a process for diagnosing disease, arising from infection with H. pylori, comprising determining from a sample isolated or derived from an individual an increased level of expression of a nucleic acid molecule having the sequence of a nucleic acid molecule set forth in the Sequence Listing. Expression of nucleic acid molecules can be measured using any one of the methods well known in the art for the quantitation of nucleic acid molecules, such as, for example, PCR, RT-PCR, Rnase protection, Northern blotting, other hybridisation methods and the arrays described herein.

Isolated as used herein means separated “by the hand of man” from its natural state; i.e., that, if it occurs in nature, it has been changed or removed from its original environment, or both. For example, a naturally occurring nucleic acid molecule or a polypeptide naturally present in a living organism in its natural state is not “isolated,” but the same nucleic acid molecule or polypeptide separated from the coexiting materials of its natural state is “isolated”, as the term is employed herein. As part of or following isolation, such nucleic acid molecules can be joined to other nucleic acid molecules, such as DNAs, for mutagenesis, to form fusion proteins, and for propagation or expression in a host, for instance. The isolated nucleic acid molecules, alone or joined to other nucleic acid molecules such as vectors, can be introduced into host cells, in culture or in whole organisms. Introduced into host cells in culture or in whole organisms, such DNAs still would be isolated, as the term is used herein, because they would not be in their naturally occurring form or environment. Similarly, the nucleic acid molecules and polypeptides may occur in a composition, such as a media formulations, solutions for introduction of nucleic acid molecules or polypeptides, for example, into cells, compositions or solutions for chemical or enzymatic reactions, for instance, which are not naturally occurring compositions, and, therein remain isolated nucleic acid molecules or polypeptides within the meaning of that term as it is employed herein.

The nucleic acids according to the present invention may be chemically synthesized. Alternatively, the nucleic acids can be isolated from H. pylori by methods known to the one skilled in the art.

According to another aspect of the present invention, a comprehensive set of novel hyperimmune serum reactive antigens and fragments thereof are provided by using the herein described antigen identification method. In a preferred embodiment of the invention, a hyperimmune serum-reactive antigen comprising an amino acid sequence being encoded by any one of the nucleic acids molecules herein described and fragments thereof are provided. In another preferred embodiment of the invention a novel set of hyperimmune serum-reactive antigens which comprises amino acid sequences selected from a group consisting of the polypeptide sequences as represented in Seq ID No 181-182, 194, 197-199, 206-207, 211-216, 219-220, 222, 226-230, 233, 235-236, 239, 241, 243, 245-246, 250, 252-253, 259, 262, 269, 272, 274-275, 279, 283-286, 290, 293-295, 297, 301-356 and fragments thereof are provided. In a further preferred embodiment of the invention hyperimmune serum-reactive antigens, which comprise amino acid sequences selected from a group consisting of the polypeptide sequences as represented in Seq ID No 186-188, 191-193, 195-196, 202, 205, 210, 217-218, 223-225, 234, 237, 240, 247-248, 251, 255, 257, 260, 263-264, 266, 268, 281, 287-288, 292, 299 and fragments thereof are provided. In a still preferred embodiment of the invention hyperimmune serum-reactive antigens which comprise amino acid sequences selected from a group consisting of the polypeptide sequences as represented in Seq ID No 183, 185, 208-209, 231, 238, 244, 254, 261, 265, 270, 277, 298 and fragments thereof are provided.

The hyperimmune serum reactive antigens and fragments thereof as provided in the invention include any polypeptide set forth in the Sequence Listing as well as polypeptides which have at least 70% identity to a polypeptide set forth in the Sequence Listing, preferably at least 80% or 85% identity to a polypeptide set forth in the Sequence Listing, and more preferably at least 90% similarity (more preferably at least 90% identity) to a polypeptide set forth in the Sequence Listing and still more preferably at least 95%, 96%, 97%, 98%, 99% or 99.5% similarity (still more preferably at least 95%, 96%, 97%, 98%, 99%, or 99.5% identity) to a polypeptide set forth in the Sequence Listing and also include portions of such polypeptides with such portion of the polypeptide generally containing at least 4 amino acids and more preferably at least 8, still more preferably at least 30, still more preferably at least 50 amino acids, such as 4, 8, 10, 20, 30, 35, 40, 45 or 50 amino acids.

The invention also relates to fragments, analogs, and derivatives of these hyperimmune serum reactive antigens and fragments thereof. The terms “fragment”, “derivative” and “analog” when referring to an antigen whose amino acid sequence is set forth in the Sequence Listing, means a polypeptide which retains essentially the same or a similar biological function or activity as such hyperimmune serum reactive antigen and fragment thereof.

The fragment, derivative or analog of a hyperimmune serum reactive antigen and fragment thereof may be 1) one in which one or more of the amino acid residues are substituted with a conserved or non-conserved amino acid residue (preferably a conserved amino acid residue) and such substituted amino acid residue may or may not be one encoded by the genetic code, or 2) one in which one or more of the amino acid residues includes a substituent group, or 3) one in which the mature hyperimmune serum reactive antigen or fragment thereof is fused with another compound, such as a compound to increase the half-life of the hyperimmune serum reactive antigen and fragment thereof (for example, polyethylene glycol), or 4) one in which the additional amino acids are fused to the mature hyperimmune serum reactive antigen or fragment thereof, such as a leader or secretory sequence or a sequence which is employed for purification of the mature hyperimmune serum reactive antigen or fragment thereof or a proprotein sequence. Such fragments, derivatives and analogs are deemed to be within the scope of those skilled in the art from the teachings herein.

Among the particularly preferred embodiments of the invention in this regard are the hyperimmune serum reactive antigens set forth in the Sequence Listing, variants, analogs, derivatives and fragments thereof, and variants, analogs and derivatives of fragments. Additionally, fusion polypeptides comprising such hyperimmune serum reactive antigens, variants, analogs, derivatives and fragments thereof, and variants, analogs and derivatives of the fragments are also encompassed by the present invention. Such fusion polypeptides and proteins, as well as nucleic acid molecules encoding them, can readily be made using standard techniques, including standard recombinant techniques for producing and expression of a recombinant polynucleic acid encoding a fusion protein.

Among preferred variants are those that vary from a reference by conservative amino acid substitutions. Such substitutions are those that substitute a given amino acid in a polypeptide by another amino acid of like characteristics. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr.

Further particularly preferred in this regard are variants, analogs, derivatives and fragments, and variants, analogs and derivatives of the fragments, having the amino acid sequence of any polypeptide set forth in the Sequence Listing, in which several, a few, 5 to 10, 1 to 5, 1 to 3, 2, 1 or no amino acid residues are substituted, deleted or added, in any combination. Especially preferred among these are silent substitutions, additions and deletions, which do not alter the properties and activities of the polypeptide of the present invention. Also especially preferred in this regard are conservative substitutions. Most highly preferred are polypeptides having an amino acid sequence set forth in the Sequence Listing without substitutions.

The hyperimmune serum reactive antigens and fragments thereof of the present invention are preferably provided in an isolated form, and preferably are purified to homogeneity.

Also among preferred embodiments of the present invention are polypeptides comprising fragments of the polypeptides having the amino acid sequence set forth in the Sequence Listing, and fragments of variants and derivatives of the polypeptides set forth in the Sequence Listing.

In this regard a fragment is a polypeptide having an amino acid sequence that entirely is the same as part but not all of the amino acid sequence of the afore mentioned hyperimmune serum reactive antigen and fragment thereof, and variants or derivative, analogs, fragments thereof. Such fragments may be “free-standing”, i.e., not part of or fused to other amino acids or polypeptides, or they may be comprised within a larger polypeptide of which they form a part or region. Also preferred in this aspect of the invention are fragments characterised by structural or functional attributes of the polypeptide of the present invention, i.e. fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta-amphipathic regions, flexible regions, surface-forming regions, substrate binding regions, and high antigenic index regions of the polypeptide of the present invention, and combinations of such fragments. Preferred regions are those that mediate activities of the hyperimmune serum reactive antigens and fragments thereof of the present invention. Most highly preferred in this regard are fragments that have a chemical, biological or other activity of the hyperimmune serum reactive antigen and fragments thereof of the present invention, including those with a similar activity or an improved activity, or with a decreased undesirable activity. Particularly preferred are fragments comprising receptors or domains of enzymes that confer a function essential for viability of H. pylori or the ability to cause disease in humans. Further preferred polypeptide fragments are those that comprise or contain antigenic or immunogenic determinants in an animal, especially in a human.

An antigenic fragment is defined as a fragment of the identified antigen, which is for itself antigenic or may be made antigenic when provided as a hapten. Therefore, also antigens or antigenic fragments showing one or (for longer fragments) only a few amino acid exchanges are enabled with the present invention, provided that the antigenic capacities of such fragments with amino acid exchanges are not severely deteriorated on the exchange(s), i.e., suited for eliciting an appropriate immune response in an individual vaccinated with this antigen and identified by individual antibody preparations from individual sera.

Preferred examples of such fragments of a hyperimmune serum-reactive antigen are selected from the group consisting of peptides comprising amino acid sequences of column “predicted immunogenic aa”, and “Location of identified immunogenic region” of Table 1, the serum reactive epitope of Table 3: especially peptides comprising amino acids 63-91, 95-101, 110-116, 134-148, 150-156, 158-164, 188-193, 197-209, 226-241, 247-254, 291-297, 312-319, 338-346, 351-358, 366-378, 404-410, 420-438, 448-454, 465-473, 482-488, 490-498, 503-510, 512-519, 531-543, 547-554, 568-575, 589-604, 610-631 and 239-308 of Seq ID No 179; 16-29, 35-47, 50-68, 70-79, 91-101, 143-149, 158-163, 185-191, 196-206, 215-224, 230-237, 244-251, 258-278, 290-311, 319-325, 338-351, 365-385, 396-429, 445-454, 458-466, 491-499, 501-521, 17-79 and 218-233 of Seq ID No 180; 4-10, 16-41, 46-66, 77-84, 91-97, 102-118, 125-144, 187-200, 202-214, 245-253, 255-261, 286-295, 300-330, 335-342, 350-361, 363-381, 385-392, 396-416, 435-450 and 460-470 of Seq ID No 181; 11-19, 27-48, 52-59, 77-82, 84-107, 118-125, 127-154, 178-183, 192-209, 215-221, 286-295, 302-313, 350-357, 402-415, 417-431, 453-463, 465-493 and 313-331 of Seq ID No 182; 19-26, 30-43, 47-55, 63-68, 72-80, 97-104, 107-119, 129-146, 160-175, 194-216, 231-251, 254-260 and 26-43 of Seq ID No 183; 7-13, 29-37, 6-81, 110-120, 123-131, 135-152, 230-249, 254-260, 284-290, 292-299, 317-326, 329-336, 403-444, 452-458, 466-477, 490-498, 510-519, 541-550, 557-566 and 533-567 of Seq ID No 184; 5-47, 71-77, 79-86, 89-95, 120-126, 137-144, 176-181, 184-196, 202-208, 211-232, 236-282, 301-313, 317-325, 341-347, 353-384, 394-400, 412-433, 436-443 and 59-75 of Seq ID No 185; 4-18, 22-38, 59-69, 106-112, 116-130, 138-149, 156-170, 175-197, 200-214, 216-223, 233-244, 255-261, 266-276, 279-286, 325-333, 342-348, 366-399, 402-420, 429-441, 1-104 and 130-147 of Seq ID No 186; 50-58, 69-95, 97-113, 131-136, 157-163, 170-175, 188-212, 220-226, 254-259, 265-277, 283-289, 297-308, 311-318, 347-358, 360-369, 378-401, 416-421, 440-450, 454-462, 470-476, 493-502, 506-514, 536-567, 585-590, 598-607, 613-618, 653-659 and 35-46 of Seq ID No 187; 16-29, 32-60, 65-87, 89-123, 128-134, 137-158, 162-173, 178-196, 210-216, 218-228 and 206-225 of Seq ID No 188; 10-20, 26-35, 51-64, 86-91, 94-100, 113-122, 154-160, 185-191, 193-201, 211-217, 225-230, 237-246, 251-257, 298-304, 306-312, 316-328, 340-348, 357-389, 391-397, 415-421, 449-456, 458-471, 488-495, 502-511, 24-55 and 236-341 of Seq ID No 189; 5-22, 41-51, 87-93, 114-122, 127-136, 150-156, 158-166, 223-233, 245-263, 291-296, 9-126 and 127-285 of Seq ID No 190; 30-43, 46-56, 61-70, 72-83, 85-93, 103-113, 119-125, 151-166, 179-191, 212-218, 225-231, 236-243, 262-267, 291-307, 331-44, 349-355, 366-372, 380-386, 414-422, 428-447, 459-464, 469-478, 507-519, 525-544, 563-569, 576-590, 620-626, 633-643, 654-659, 665-671, 684-707, 717-723, 725-733, 747-779, 782-801 and 347-361 of Seq ID No 191; 4-12, 14-26, 37-80, 107-115, 133-139, 144-150, 154-165, 173-180, 191-199, 205-211, 221-231, 237-244, 254-284, 307-340, 342-353, 360-368, 370-380, 479-493, 495-503, 509-522, 525-536, 539-547, 554-560, 565-573, 578-583, 7-23 and 465-479 of Seq ID No 192; 4-17, 47-55, 76-83, 85-100, 104-112, 117-123, 126-135, 142-148, 156-167, 174-182, 267-273 and 258-283 of Seq ID No 193; 8-32, 36-42, 65-88, 102-108, 112-140, 147-163, 170-179, 183-193 and 117-124 of Seq ID No 194; 12-18, 45-50, 62-77, 82-95, 99-113, 115-123, 125-147, 155-177, 187-209, 211-223, 244-253, 259-270, 278-297, 302-307, 311-318, 329-334, 350-356, 359-365, 390-400, 402-413 and 333-350 of Seq ID No 195; 4-13, 15-27, 30-46, 53-58, 68-74, 82-95, 115-126, 134-139, 148-153, 159-176, 182-199, 201-217, 220-225, 227-235, 237-248, 253-266, 300-315, 322-336, 390-396, 412-426, 438-445, 448-459, 477-484, 502-508, 515-527, 529-537, 553-568, 643-651, 658-667, 690-703 and 376-400 of Seq ID No 196; 4-10, 24-32, 38-55, 59-67, 70-77, 80-87, 89-97, 123-129, 134-151, 166-172, 178-189, 191-216, 218-235, 245-259, 271-315, 326-339, 341-360 and 73-94 of Seq ID No 197; 13-25, 31-38, 43-57, 79-85, 92-99, 106-112, 117-128, 130-139, 146-158, 160-175, 194-204, 211-222, 225-232, 234-242, 263-270, 278-292, 299-320, 322-333 and 240-256 of Seq ID No 198; 4-17, 55-63, 66-101, 109-131, 135-143, 145-151, 155-161, 164-170, 177-185, 192-198, 213-218, 223-238, 246-256, 258-268, 273-283, 309-314, 322-328 and 195-221 of Seq ID No 199; 13-24, 31-39, 41-50, 63-69, 90-96, 104-109, 116-141, 148-153, 161-167, 173-178, 190-209, 253-258, 265-272, 279-289, 295-312, 317-343, 355-366, 376-389, 400-407, 430-451, 453-464, 466-472, 487-493, 499-505, 523-538, 554-559, 568-579, 584-601 and 344-363 of Seq ID No 200; 5-22, 30-36, 53-59, 61-70, 82-92, 99-106, 120-131, 135-148, 154-167, 169-183, 187-199, 204-212, 231-247 and 111-249 of Seq ID No 201; 17-36, 40-66, 71-144, 148-171, 173-191, 199-214, 220-252, 265-272, 278-288, 298-333, 342-385 and 287-307 of Seq ID No 202; 4-16, 22-28, 30-36, 42-48, 95-116, 154-162, 164-174, 239-252, 258-263, 273-285, 306-313, 323-333, 341-357, 363-369, 372-379, 395-401, 430-436, 438-453, 464-480, 33-44, 233-258 and 349-369 of Seq ID No 203; 4-21, 30-37, 46-53, 59-68, 80-92, 98-104, 118-143, 150-160, 165-185, 187-200, 204-211, 224-236, 241-246, 252-258, 271-280, 288-294, 311-320, 335-341 and 191-350 of Seq ID No 204; 4-16, 37-59, 64-70, 79-87, 93-102, 107-127, 143-165, 172-188, 197-204, 207-218, 221-227, 242-248, 258-277, 289-296, 298-316, 332-338, 344-365, 367-373, 375-382, 400-408, 415-425, 438-446 and 235-250 of Seq ID No 205; 4-37, 39-66, 84-98, 101-127, 140-149, 157-163, 166-172, 175-182, 184-193, 203-208, 215-232, 234-247, 250-299, 303-345 and 183-204 of Seq ID No 206; 10-20, 41-61, 73-87, 112-141, 176-192, 194-201, 205-222, 230-237, 257-264, 276-282, 284-310, 312-318, 330-337, 349-357 and 304-328 of Seq ID No 207; 4-31, 42-103, 105-113, 121-153, 160-181, 188-196, 210-226, 231-264, 272-287, 297-304, 328-336 and 304-318 of Seq ID No 208; 21-43, 46-52, 54-70, 72-79, 94-107, 133-141, 160-166, 217-253, 311-317, 359-365, 374-381, 390-395, 434-440, 488-494, 497-502, 511-522, 554-563, 565-574, 577-585, 591-598, 601-606, 617-625, 633-643, 658-664, 676-682, 694-702, 710-719, 754-760, 782-788, 802-808, 916-921, 942-948, 955-964, 973-979, 992-998, 1006-1011, 1016-1023, 1030-1038, 1046-1053, 1059-1066, 1088-1098, 1119-1126, 1129-1135, 1156-1171, 1173-1181, 1202-1210, 1255-1261, 1268-1280, 1295-1310, 1312-1320, 1375-1381, 1406-1417, 1450-1471, 1478-1492, 1498-1506, 1569-1578, 1603-1608, 1611-1624, 1648-1655, 1663-1670, 1680-1698, 1702-1707, 1713-1719, 1737-1742, 1747-1753, 1762-1769, 1771-1785, 1790-1804, 1811-1818, 1830-1836, 1838-1852, 1874-1886, 1893-1899, 1902-1909, 1942-1948, 1952-1962, 1980-1986, 2001-2017, 2020-2028, 2042-2050, 2052-2068, 2074-2079, 2083-2095, 2107-2113, 2147-2155, 2177-2194, 2203-2211, 2236-2241, 2251-2258, 2267-2274, 2285-2292, 2314-2328, 2330-2340, 2358-2365, 2390-2401, 2408-2418, 2432-2453, 2463-2476, 2486-2507, 2528-2537, 2540-2548, 2552-2558, 2568-2576, 2596-2601, 2610-2622, 2629-2638, 2653-2669, 2718-2727, 2749-2767, 2777-2784, 2789-2795, 2806-2815, 2817-2824, 2835-2843, 2847-2854, 2860-2881, 511-523, 612-630 and 1790-1803 of Seq ID No 209; 4-54, 61-68, 72-82, 86-93, 100-108, 115-130, 147-154, 187-194, 196-207, 224-229, 236-251, 275-287 and 96-109 of Seq ID No 210; 31-39, 62-69, 91-101, 158-172, 175-180, 186-193, 201-208, 210-223, 243-250, 273-286, 293-299, 319-325, 343-354, 356-365, 368-384, 414-435, 471-491, 512-518, 550-556, 567-581, 584-589, 633-639, 680-692, 697-708, 716-721, 747-754, 779-786, 810-816 and 366-503 of Seq ID No 211; 5-20, 22-48, 57-65, 96-101, 111-122, 130-145, 154-164, 170-181, 193-199, 201-216, 224-241, 244-262, 281-323, 342-351, 359-367, 369-396, 406-416, 424-433, 450-456, 485-491, 493-499, 501-515, 517-535 and 289-305 of Seq ID No 212; 4-17, 22-44, 53-60, 66-83, 87-94, 101-106, 110-116, 131-137, 148-183, 189-207, 209-215, 233-242, 251-262, 264-272, 290-296, 308-327, 359-373, 375-380, 397-405, 415-420, 426-433, 444-475, 478-484, 529-536, 548-558 and 106-126 of Seq ID No 213; 4-38, 42-50, 58-64, 72-81, 92-118, 140-146, 157-165, 172-192, 198-204, 208-216, 227-234, 238-258, 271-278, 288-293, 311-322, 327-346, 357-370, 375-383, 395-409, 411-417, 425-432, 436-445, 109-129 and 370-380 of Seq ID No 214; 23-30, 36-49, 52-64, 86-94, 97-104, 121-129, 257-272, 279-286, 288-294, 307-327, 334-340, 369-375, 377-386, 406-412, 418-423, 430-438, 441-447, 459-465, 469-476, 482-488, 510-546, 550-580, 584-622, 638-645, 653-659, 675-683, 692-705, 723-731, 752-761, 788-795 and 54-72 of Seq ID No 215; 11-33, 36-46, 88-104, 116-126, 134-170, 189-195, 199-217, 225-250, 255-261, 266-273, 280-291, 296-313, 334-341, 343-349, 354-360, 362-369, 373-380, 387-401, 406-420 and 259-273 of Seq ID No 216; 9-14, 28-44, 57-64, 72-79, 86-93, 104-111, 116-126, 142-150, 159-164 and 61-86 of Seq ID No 217; 10-17, 26-33, 43-61, 69-95, 101-107, 109-125, 129-135, 137-144, 147-153, 158-169, 177-187, 209-219, 221-232, 235-247, 261-268, 271-282, 296-302, 306-347, 355-362, 364-379, 386-399, 409-418, 424-442, 451-460, 467-479, 490-498 and 60-74 of Seq ID No 218; 8-14, 20-31, 65-84, 94-99, 154-179, 193-207, 238-253 and 96-118 of Seq ID No 219; 4-24, 30-44, 47-62, 84-93, 108-116, 124-133, 136-141, 201-209, 217-223, 228-235, 238-245, 247-270, 275-285, 290-314, 328-338, 342-349, 353-365, 375-383, 386-392, 394-402, 417-427, 443-459, 465-481, 492-514, 516-524, 550-566, 602-617, 630-639, 666-676, 687-693, 719-730, 747-753, 783-790, 799-816, 824-831, 837-842 and 167-189 of Seq ID No 220; 6-15, 18-28, 58-66, 84-101, 106-129, 136-151, 154-165, 182-203, 205-211, 214-220, 222-228, 233-240, 251-260, 270-277, 284-291, 306-315, 322-328, 363-369, 378-388, 392-405, 443-452, 495-501, 512-523, 574-583 and 362-375 of Seq ID No 221; 5-25, 27-34, 47-59, 64-70, 76-86, 145-158, 166-183, 189-202, 217-231, 235-242, 260-270, 278-309 and 1-102 of Seq ID No 222; 4-19, 24-76, 78-83, 90-99, 102-109, 114-122, 137-147, 154-174, 177-188, 203-212, 217-223, 227-239 and 226-325 of Seq ID No 223; 7-37, 71-90, 94-109, 117-128, 141-153, 179-192, 199-206, 225-231, 237-243, 258-264 and 40-51 of Seq ID No 224; 13-19, 25-30, 46-59, 75-91, 101-107, 114-124, 129-135, 137-145, 160-167, 171-179, 187-194, 209-215, 217-222, 229-239, 243-249, 257-265, 269-275, 299-308, 310-327 and 282-300 of Seq ID No 225; 86-100, 216-230, 342-369, 382-388, 424-430, 438-445, 452-458, 488-494, 501-518, 554-560, 568-574, 584-592, 603-609, 611-629, 639-645, 652-661, 669-699, 708-714, 726-738, 747-753, 763-775, 785-791, 794-807, 815-824, 826-845, 854-860, 863-868, 870-883, 892-898, 901-906, 909-921, 930-937, 946-959, 968-974, 977-990, 998-1007, 1009-1027, 1037-1043, 1046-1051, 1053-1066, 1075-1081, 1084-1089, 1092-1103, 1113-1119, 1122-1135, 1143-1152, 1154-1172, 1182-1188, 1191-1196, 1200-1210, 1220-1226, 1229-1235, 1237-1249, 1259-1265, 1268-1281, 1289-1298, 1305-1318, 1328-1334, 1337-1343, 1345-1357, 1367-1373, 1390-1396, 1405-1411, 1418-1423, 1426-1435, 1445-1455, 1474-1483, 1493-1500, 1505-1512, 1517-1524, 1538-1544, 1568-1578, 1595-1601, 1674-1682, 1687-1720, 1728-1736, 1738-1744, 1754-1761, 1764-1774, 1798-1824, 1836-1842, 1886-1893, 1895-1903, 366-781, 782-1518 and 1731-1747 of Seq ID No 226; 4-17, 20-39, 46-55, 60-66, 102-110, 114-122, 125-131, 161-167, 172-178, 185-190, 195-202, 218-232, 236-252, 264-291, 293-302, 309-315, 324-339 and 169-381 of Seq ID No 227; 5-10, 13-40, 42-53, 69-75, 83-89, 120-135, 150-161, 174-190, 203-225, 229-247, 257-287, 318-348 and 30-200 of Seq ID No 228; 7-19, 43-53, 64-72, 124-139, 52-84 and 120-131 of Seq ID No 229; 12-19, 39-48, 58-100, 117-123, 154-162, 164-187, 189-195, 202-216, 218-235, 241-246, 262-278, 315-328, 333-347, 354-366, 372-379, 391-405, 422-429, 431-442, 444-450, 458-466, 478-485, 494-501, 504-510, 520-535, 573-580, 589-598, 615-625, 666-676, 686-698, 722-729, 737-746, 756-767, 787-796, 805-816, 824-829, 833-848, 856-864, 866-876, 879-886, 898-904, 918-924, 927-934, 941-960, 967-978 and 561-575 of Seq ID No 230; 11-29, 49-55, 70-77, 84-100, 102-112, 148-155, 160-177, 181-204 and 1-104 of Seq ID No 231; 27-44, 64-71, 122-133, 151-156, 164-178, 214-220, 226-232, 235-244, 253-262, 282-288, 294-310, 317-325, 350-356, 362-368, 376-383, 438-443, 449-454, 459-464, 492-498, 500-511, 529-535, 538-546, 567-573, 597-603, 660-665, 674-679, 724-734, 763-769, 773-784, 791-801, 807-815, 821-826, 840-848, 863-868, 897-902, 908-928, 932-953, 956-975, 980-987, 990-996, 1012-1018, 1042-1063, 1095-1116, 1149-1157, 1160-1167, 110-357, 358-501 and 502-1161 of Seq ID No 232; 4-21, 64-71, 73-84, 128-138, 144-162, 203-217, 240-263, 288-298, 300-308, 310-317, 325-351, 369-380, 391-411 and 330-345 of Seq ID No 233; 5-11, 25-31, 39-48, 51-79, 89-98, 100-122, 135-148, 166-201, 203-227, 230-250, 254-260, 266-272, 274-282, 299-305, 328-337 and 31-45 of Seq ID No 234; 12-23, 29-48, 51-60, 66-72, 75-81, 83-93, 103-115, 133-148, 168-174, 195-204, 222-229, 231-240, 242-251, 270-280, 286-305, 322-344, 349-360, 364-370, 378-400, 421-441, 448-484, 486-493, 495-501, 504-534, 547-561, 567-590, 597-607, 621-635, 643-649, 658-685, 688-694, 702-711, 717-731, 737-742, 759-765, 767-772, 776-786, 803-809, 815-825, 854-908, 910-919, 923-930, 942-948, 961-975, 994-1014 and 915-940 of Seq ID No 235; 4-9, 32-47, 51-61, 75-96, 139-191 and 1-124 of Seq ID No 236; 4-13, 17-38, 43-49, 55-76, 88-95, 110-121, 128-146, 151-157, 162-214, 222-240, 243-249, 251-273, 275-281, 292-298, 300-309, 312-320, 322-331, 355-369, 376-408, 446-460, 471-482, 485-509 and 191-203 of Seq ID No 237; 4-21, 72-82, 89-103, 106-115, 118-124, 140-146, 174-184, 191-200, 204-213, 218-224, 261-266, 282-293, 299-309, 311-340, 342-358, 362-372, 381-389, 391-402, 413-421, 438-447, 457-464, 470-478, 501-507, 545-560, 578-624, 631-641, 658-670, 680-689, 717-738, 753-759, 795-805, 816-822, 830-838, 842-848, 869-881, 892-898, 33-51 and 818-835 of Seq ID No 238; 4-21, 79-85, 156-177, 183-188, 206-214, 243-249, 261-269, 287-292, 315-322, 334-345, 360-366, 374-390, 402-411, 37-97 and 260-399 of Seq ID No 239; 4-9, 19-54, 58-78, 97-104, 111-120, 126-134, 137-145, 163-173, 178-188, 193-203, 211-224, 246-286, 288-324, 337-346, 355-362, 374-390, 392-398, 409-417 and 240-249 of Seq ID No 240; 5-12, 14-31, 35-41, 43-61, 82-92, 97-105, 134-145, 155-166, 184-203, 215-223, 225-251, 272-279, 281-306, 310-345, 358-418, 435-473, 482-490, 525-532, 538-547, 549-563, 578-604, 613-639 and 144-154 of Seq ID No 241; 53-59, 64-72, 74-100, 133-152, 154-172, 176-181, 207-214, 225-238, 275-297, 304-310, 331-340, 362-367, 384-395, 403-410, 437-443, 448-456, 482-490, 579-597, 602-610, 625-630, 633-651, 699-707, 709-715, 734-743, 750-762 and 544-685 of Seq ID No 242; 12-18, 22-40, 45-83, 89-97, 103-109, 147-153, 159-173, 195-204, 210-219, 243-253, 259-265, 273-282, 303-309, 315-325, 332-340, 346-358, 362-367, 377-390, 393-402, 418-426, 447-455, 467-480, 505-512, 514-525, 548-561, 566-576, 584-596, 619-626, 638-645, 649-659, 661-680, 699-708, 714-720, 753-759, 766-772, 775-781, 801-808, 202-218, 282-299, 339-350 and 617-628 of Seq ID No 243; 5-33, 52-62, 87-101, 111-135, 137-143, 145-152, 190-202, 209-221, 233-245, 253-270 and 151-215 of Seq ID No 244; 19-29, 32-39, 42-48, 75-94, 124-135, 137-145, 152-160, 176-182, 193-203, 215-236, 266-273, 275-291, 297-306, 311-319, 322-342, 348-360, 369-378, 394-401 and 48-64 of Seq ID No 245; 4-11, 13-33, 36-43, 53-63, 65-80, 112-129, 134-141, 143-155, 157-168, 178-188, 191-199, 201-207, 215-229, 242-255, 263-270, 283-315, 320-329, 333-338, 340-349, 412-426, 465-478, 485-490, 498-512, 540-554 and 390-516 of Seq ID No 246; 4-18, 23-32, 41-47, 54-70, 88-99, 104-111, 118-138, 143-148, 150-162, 168-175, 181-188, 203-211, 214-220, 227-245, 251-268, 275-281, 287-296, 323-333 and 1-90 of Seq ID No 247; 8-34, 38-49, 72-83, 85-91, 94-104, 112-125, 134-142, 148-168, 181-189, 191-198, 202-214, 222-233, 242-254, 256-262, 273-278, 287-294, 314-325 and 141-159 of Seq ID No 248; 4-24, 30-36, 47-75, 82-105, 124-134, 151-157, 192-202, 208-214, 219-226, 234-247, 285-290, 318-324, 332-340, 343-349, 380-386, 453-462, 472-478, 484-501, 531-540, 550-557, 604-612, 620-625, 642-648, 652-671, 64-84, 93-180 and 181-446 of Seq ID No 249; 12-18, 24-32, 68-75, 77-83, 96-101, 109-116, 129-136, 152-164, 175-184, 190-199, 206-215, 224-233, 241-250, 258-264, 273-292, 302-312, 319-331, 334-346, 348-368, 387-395, 408-416, 420-429, 437-452 and 364-374 of Seq ID No 250; 11-28, 36-52, 60-67, 74-79, 108-116 and 61-76 of Seq ID No 251; 20-27, 38-49, 69-74, 84-107, 138-145, 161-168, 179-195, 210-226, 228-252, 267-281, 283-296, 305-311, 333-340, 342-356, 361-372, 380-399, 401-414, 458-466, 475-481, 492-507, 515-520 and 146-160 of Seq ID No 252; 43-61, 68-74, 76-90, 120-128, 130-149, 156-161, 164-182, 206-234, 242-252, 269-274, 291-304, 332-345, 349-355, 360-371, 374-388, 434-440, 447-453, 459-465, 469-496, 504-522 and 261-285 of Seq ID No 253; 4-17, 24-30, 37-49, 87-98, 118-124, 126-136, 144-171, 176-188, 206-214, 216-228, 233-240, 246-252, 262-271, 277-297, 307-330, 333-342, 346-352, 355-361, 368-386, 391-400, 413-420, 474-480 and 401-427 of Seq ID No 254; 15-26, 31-46, 51-72, 80-93, 96-109, 131-137, 150-158, 179-185, 189-209, 211-219, 221-234, 241-247, 255-262, 265-271, 283-288 and 173-190 of Seq ID No 255; 28-37, 39-45, 51-58, 77-84, 89-97, 132-148, 171-180, 199-205, 212-218, 220-226, 257-265, 273-300, 307-327, 334-340, 344-365, 385-390, 402-408, 426-436, 450-468, 476-485 and 425-497 of Seq ID No 256; 4-25, 70-76, 80-88, 90-100, 120-128, 162-169, 183-203, 261-277, 279-289, 291-297, 302-308, 321-327, 339-353, 358-377, 392-401, 404-410, 414-422, 443-450, 456-461, 470-488, 490-497, 510-535, 570-611, 618-630, 639-647, 649-660, 668-690, 702-716, 718-724, 737-747, 750-764 and 497-509 of Seq ID No 257; 12-48, 50-64, 99-108, 216-223, 235-241, 244-254, 262-274, 287-293, 310-316, 320-326, 361-366, 377-383, 390-395, 408-414, 418-425, 438-444, 462-469, 494-505, 524-530, 536-547, 551-566, 592-598, 601-613, 678-685, 687-695, 709-717, 727-737, 751-757, 760-765, 772-778, 782-788, 801-807, 822-830, 859-868, 870-878, 884-890, 898-903, 909-919, 953-969, 973-980, 990-1000, 1002-1019, 1041-1047, 1059-1065, 1090-1095, 1116-1127, 1130-1139, 1143-1149, 1151-1168, 1178-1183, 1188-1195, 1197-1209, 1213-1220, 1226-1234, 1236-1247, 1255-1274, 1276-1282, 76-100, 270-284, 309-438, 493-505, 786-942 and 947-967 of Seq ID No 258; 4-9, 24-34, 46-95, 97-109, 119-130 and 138-156 of Seq ID No 259; 9-26, 28-35, 43-53, 55-68, 83-92, 99-105, 110-135, 139-149, 157-162, 164-170, 173-183, 193-208, 210-230, 239-245, 253-259, 263-271, 293-305, 310-320, 322-331, 336-343, 351-364, 367-376, 92-107 and 154-173 of Seq ID No 260; 19-39, 52-62, 108-117, 145-152, 160-168, 194-203, 229-240, 252-268, 280-287, 308-316, 333-339, 383-390, 403-412, 414-424, 438-445, 464-472, 479-484, 489-505, 510-526 and 247-260 of Seq ID No 261; 5-17, 25-52, 60-77, 105-113, 118-125, 162-167, 228-234, 272-279, 328-334, 341-357, 381-395, 400-406, 512-518, 557-569, 586-592, 645-651, 690-695, 701-709, 720-726, 733-743, 751-758, 781-786, 879-886, 929-934, 939-944, 952-960, 965-975, 994-1001, 1039-1045, 1102-1109, 1164-1181, 1198-1206, 1223-1229, 1253-1259, 1283-1292, 1312-1317, 1339-1349, 1360-1370, 1389-1398, 1400-1412, 1452-1465, 1470-1484, 1490-1497, 1519-1525, 1554-1564, 1578-1591, 1623-1636, 1638-1646, 1669-1679, 1685-1697, 1704-1711, 1713-1720, 1730-1736, 1738-1749, 1756-1764, 1778-1786, 1796-1803, 1817-1826, 1849-1866, 1975-1993, 2017-2032, 2044-2053, 2070-2086, 2091-2109, 2116-2127, 2156-2167, 2182-2188, 2197-2202, 2244-2252, 2281-2287, 2290-2307, 2350-2361, 2383-2404, 2425-2433, 2445-2455, 2495-2505 and 394-549 of Seq ID No 262; 9-24, 31-53, 57-67, 69-79, 84-114, 133-141, 144-172, 178-186 and 13-46 of Seq ID No 263; 4-25, 27-35, 43-52, 59-70, 79-91, 115-130, 136-152, 154-163, 170-179 and 1-58 of Seq ID No 264; 4-30, 49-55, 71-80, 96-105, 111-126, 139-146, 149-162, 239-245, 279-285, 290-296, 300-307, 331-337, 343-350 and 250-351 of Seq ID No 265; 9-27, 34-41, 43-51, 92-111, 114-120, 123-131, 139-150, 156-171, 176-186, 188-204, 229-241, 252-258, 266-279, 288-297, 319-334, 338-348, 373-379, 389-398, 431-439, 479-484 and 214-398 of Seq ID No 266; 4-15, 18-27, 47-52, 68-83, 91-97, 104-110, 115-121, 139-147, 157-164, 198-206, 227-236, 241-254, 264-273, 278-289, 311-320, 353-361, 372-383, 405-420, 426-434 and 232-386 of Seq ID No 267; 4-10, 24-34, 91-97, 129-141, 156-163, 184-190, 205-219, 229-235, 256-273, 278-285 and 93-116 of Seq ID No 268; 7-29, 35-54, 71-83, 85-91, 104-111, 122-134, 138-144, 146-154, 158-174, 177-183, 186-201, 207-215, 223-235, 240-247, 262-273, 275-283, 287-292 and 48-66 of Seq ID No 269; 7-27, 31-47, 49-70, 75-102, 110-149, 157-171, 217-223, 235-251, 294-302, 358-364, 367-375, 387-393, 395-412, 423-430, 441-451, 456-470, 472-486, 488-495, 499-509, 515-529, 536-549, 556-570, 574-603, 607-615, 625-633, 642-658, 670-676, 683-702, 708-716, 720-726, 747-756, 763-784, 803-812, 815-826 and 475-490 of Seq ID No 270; 7-22, 30-38, 53-59, 64-75, 83-95, 97-112, 120-131, 133-142, 145-151, 154-166, 172-180, 189-203, 227-238, 277-287, 9-156 and 174-287 of Seq ID No 271; 13-23, 25-32, 111-117, 150-164, 185-193, 207-212, 216-224, 230-236, 263-272, 304-311, 342-348, 374-385, 391-407, 444-458, 480-487, 489-499, 523-542, 544-558, 572-579, 620-640, 686-696, 703-710, 742-755, 765-772, 817-822, 830-837, 865-872, 931-937 and 66-86 of Seq ID No 272; 4-27, 49-56, 62-70, 86-92, 121-127, 151-163, 170-182, 195-202, 212-226, 237-243 and 234-254 of Seq ID No 273; 4-10, 13-24, 39-51, 62-78, 92-104, 107-117, 134-141, 156-161, 166-181, 210-216, 222-229, 256-266, 273-280, 297-304, 313-330, 336-349, 371-376, 433-439, 443-448, 488-493, 506-515, 527-534, 560-572, 575-583, 587-593 and 252-483 of Seq ID No 274; 4-15, 21-38, 45-56, 81-95, 102-108, 118-130, 133-147, 152-162, 166-171, 199-204, 211-218, 230-240, 253-261, 274-283, 288-294, 312-317, 325-336, 344-357, 391-414 and 24-146 of Seq ID No 275; 26-31, 38-56, 65-82, 90-101, 112-119, 123-153, 175-188, 197-216, 234-242, 249-265, 273-286, 290-305, 327-335, 338-346, 361-372, 394-404 and 290-306 of Seq ID No 276; 17-26, 43-48, 50-73, 81-93, 95-107, 139-146, 158-168, 171-176, 190-196, 202-212, 216-223, 243-266, 274-282, 308-313, 324-330, 344-378, 380-387, 403-422, 427-443, 448-455, 457-465, 491-515, 517-528, 553-567, 589-599, 610-617, 642-648, 670-697, 709-717, 726-743, 745-759, 769-803, 807-823, 840-849 and 820-851 of Seq ID No 277; 4-18, 39-48, 53-63, 66-90, 102-117, 125-134, 137-145, 156-162, 169-197, 26-40 and 56-80 of Seq ID No 278; 21-33, 36-42, 49-60, 68-76, 91-105, 123-130, 141-161, 169-178, 185-190, 192-199, 205-214, 223-233, 239-247, 260-269, 284-293, 300-314, 324-352, 357-364, 373-382, 389-403, 420-432, 438-446, 466-471, 477-484, 503-509, 549-556, 558-576, 600-623, 625-635, 654-661, 663-669, 671-687, 702-716, 735-741, 744-750, 757-766, 776-786, 807-815, 824-832, 854-860, 863-897, 909-915, 920-946, 952-959, 982-997, 1024-1038, 1049-1055, 1071-1085, 1104-1113, 1121-1132, 1138-1150, 1187-1196, 1212-1221, 1227-1236, 1257-1262, 1264-1278, 1282-1294, 1307-1318, 1353-1370, 1382-1388, 1396-1409, 1434-1440, 1446-1454, 1465-1478, 1485-1513, 1516-1529, 1540-1545, 1563-1568, 1575-1593, 1607-1616, 1628-1645, 1648-1661, 1676-1682, 1689-1697, 1713-1719, 1739-1749, 1753-1758, 1763-1774, 1797-1803, 1807-1846, 1855-1874, 1877-1891, 1893-1907, 1912-1925, 1931-1943, 1955-1965, 1976-1990, 2032-2043, 2045-2051, 2099-2105, 2131-2138, 2161-2179, 2188-2199, 2205-2216, 2219-2227, 2235-2245, 2247-2267, 2277-2288, 2294-2304, 2314-2326, 2346-2358, 2365-2377, 2383-2402, 2407-2423, 2437-2450, 2454-2473, 2489-2497, 2525-2531, 2557-2570, 2580-2587, 2589-2599, 2621-2641, 2647-2653, 2661-2677, 2685-2690, 2697-2717, 2722-2733, 2739-2777, 2786-2793, 2801-2808, 2811-2822, 2825-2835, 2838-2845, 2859-2871, 2877-2883, 213-344, 954-1080 and 2524-2733 of Seq ID No 279; 10-16, 18-23, 28-41, 63-69, 77-91, 101-109, 118-136, 146-153, 155-162, 168-179, 192-207, 217-226, 229-235, 239-254, 279-286, 294-307, 313-319, 334-341, 344-353, 363-377, 390-396 and 178-328 of Seq ID No 280; 18-42, 68-84, 89-95, 100-105, 107-115, 125-135, 154-177, 189-195, 205-228, 236-243, 252-259, 279-300, 309-316, 323-331, 340-351, 353-364, 377-402 and 85-97 of Seq ID No 281; 4-18, 26-32, 66-76, 100-126, 151-159, 178-186, 188-194, 200-210, 241-248, 253-259, 262-279, 284-291, 307-313, 315-322, 327-337, 376-386, 399-407, 432-441, 467-473, 487-497, 499-505, 543-549, 560-568, 585-593, 598-604, 608-614, 630-642, 647-653, 690-703, 717-730, 21-200 and 468-480 of Seq ID No 282; 17-49, 52-58, 62-73, 78-97, 100-117, 122-172, 185-190, 193-217, 225-236 and 33-42 of Seq ID No 283; 7-39, 50-58, 73-89, 96-107, 109-120, 126-142, 152-170, 178-202, 205-211, 224-244, 249-259, 261-270, 300-310, 312-325 and 158-169 of Seq ID No 284; 4-31, 40-64, 71-82, 85-92, 102-124, 126-139, 147-152, 159-173, 176-188, 195-207, 210-216, 234-241, 249-256, 258-276, 279-293, 296-302, 310-315, 349-356, 363-378, 380-403, 411-426, 435-441, 448-459, 463-476, 488-494 and 201-221 of Seq ID No 285; 5-13, 15-74, 87-104, 107-120, 123-129, 136-145, 150-191, 193-206, 227-248, 250-264, 278-302, 304-323, 332-378, 384-407, 409-419, 425-457, 462-471, 474-497, 511-545, 555-564, 571-578, 585-598, 640-647, 669-675, 682-691, 693-705, 729-743, 752-761, 772-780, 786-804, 808-818, 822-846, 858-880, 884-900, 910-939, 941-947, 962-971, 973-988, 998-1003, 1007-1027 and 236-259 of Seq ID No 286; 4-19, 27-8, 81-111, 121-160 and 60-79 of Seq ID No 287; 4-37, 40-46, 52-57, 199-205, 222-229, 236-244, 250-267, 269-282 and 27-197 of Seq ID No 288; 4-16, 24-30, 32-38, 63-75, 86-92, 98-111, 113-126, 160-165, 170-180, 198-204, 227-233, 239-245, 253-273, 308-314, 352-365, 382-387, 395-403, 423-429, 472-482, 484-493, 501-507, 518-526, 536-541, 543-550, 556-562, 586-600, 626-633, 649-661, 680-688 and 546-559 of Seq ID No 289; 16-33, 48-59, 63-71, 77-92, 94-109, 117-124, 139-151, 169-181, 184-227, 233-249, 251-261, 263-275, 282-294, 297-321, 326-332, 341-355, 383-399 and 258-272 of Seq ID No 290; 11-26, 31-39, 43-52, 55-62, 64-70, 80-94, 123-133, 135-141, 172-181, 185-206, 209-218, 224-230, 238-244, 251-262, 264-271, 290-301, 306-324, 333-340, 350-357, 367-375, 390-397, 434-441, 443-448, 77-226 and 350-429 of Seq ID No 291; 4-13, 22-27, 31-45, 50-59, 72-96, 99-114, 131-141, 143-150, 159-176, 180-186, 189-198, 208-214, 234-253, 271-287, 294-299, 310-366, 382-390, 398-416, 424-443 and 283-305 of Seq ID No 292; 9-26, 30-53, 62-72, 86-95, 112-122, 136-145, 153-160, 209-221, 227-237, 241-268, 281-288, 291-298, 308-314, 321-328, 336-346, 351-379, 388-397, 409-416, 423-433, 443-481, 511-519 and 213-232 of Seq ID No 293; 12-18, 25-31, 38-50, 59-67, 71-82, 96-126 and 76-88 of Seq ID No 294; 4-25, 39-44, 64-71, 74-88, 100-113, 128-138, 151-162, 164-177, 185-190, 204-213, 233-239, 246-254, 281-286, 293-306, 309-318, 333-347, 349-359, 385-398, 404-423, 458-465, 477-484, 490-499, 501-533, 554-566, 582-590, 596-616, 624-629, 631-639, 654-680, 694-720, 735-743 and 2-100 of Seq ID No 295; 4-16, 36-41, 52-75, 98-107, 109-117, 122-128, 133-139, 141-155, 159-165, 169-182, 187-193, 195-201, 211-224, 230-236, 247-269, 278-290 and 75-92 of Seq ID No 296; 7-21, 25-33, 37-43, 87-94, 103-120, 131-147, 168-174, 197-203, 207-212, 227-237, 247-257, 263-271, 279-287, 298-306, 320-325, 332-340, 363-374, 379-384, 390-401, 403-414, 428-433, 448-457, 462-475, 483-390, 513-519, 525-535, 543-554, 559-566, 571-620, 625-631, 636-642, 659-670, 688-706, 708-723, 770-779, 787-793, 796-807, 820-840, 848-854, 863-874, 895-905, 912-919, 934-942, 968-975, 983-1000, 1012-1019, 1026-1036, 1050-1060, 1064-1070, 1081-1091, 1094-1108, 1112-1118, 1140-1152, 1164-1169, 1172-1180, 1187-1192 and 732-748 of Seq ID No 297; 23-40, 42-59, 66-73, 78-97, 111-128, 130-141, 157-166, 178-183 and 53-71 of Seq ID No 298; 4-27, 38-44, 47-57, 59-85, 99-106, 114-121, 154-166, 181-186, 193-198, 238-244, 253-262, 272-278, 287-299, 314-320, 338-350, 358-368, 382-388, 407-416, 433-446, 456-461, 463-473 and 86-195 of Seq ID No 299; 5-24, 38-59, 64-80, 87-99, 105-126, 134-142, 149-163, 165-179, 181-202, 205-220, 227-233, 243-250, 257-263 and 87-245 of Seq ID No 300; 5-32, 47-53, 66-79, 81-97, 115-151, 155-174, 183-188, 196-210, 215-226, 230-238, 253-258, 263-270, 276-282, 295-301, 304-325, 334-344, 360-390, 397-412, 425-432, 434-462, 478-494, 508-526, 539-564, 571-579, 347-371 and 375-386 of Seq ID No 301; 4-15, 36-44, 49-56, 60-66, 68-82, 84-103, 109-115, 118-141, 147-154, 160-168, 176-185 and 26-39 of Seq ID No 302; 7-13, 23-33 and 13-21 of Seq ID No 303; 2-10 of Seq ID No 304; 4-9, 12-18, 35-42, 49-62 and 6-18 of Seq ID No 305; 19-25 and 1-13 of Seq ID No 306; 15-21, 27-45 and 12-25 of Seq ID No 307; 14-20 and 1-14 of Seq ID No 308; 4-18 and 13-26 of Seq ID No 309; 8-21 and 2-20 of Seq ID No 310; 4-14 and 4-16 of Seq ID No 311; 3-12 of Seq ID No 312; 6-14, 6-25, 35-57 and 2-14 of Seq ID No 313; 6-25, 35-57 and 17-31 of Seq ID No 314; 14-25, 32-46 and 5-19 of Seq ID No 315; 18-31 and 5-16 of Seq ID No 316; 19-24 and 4-26 of Seq ID No 317; 13-21, 29-34, 47-58, 61-73 and 36-47 of Seq ID No 318; 4-15 and 5-24 of Seq ID No 319; 6-18 of Seq ID No 320; 13-20 and 4-13 of Seq ID No 321; 15-23 of Seq ID No 322; 4-9 and 7-21 of Seq ID No 323; 1-10 of Seq ID No 324; 4-14 of Seq ID No 325; 4-17, 35-41, 46-89, 93-98 and 70-88 of Seq ID No 326; 1-13 of Seq ID No 327; 4-16, 26-32 and 25-38 of Seq ID No 328; 8-15, 23-28 and 4-17 of Seq ID No 329; 4-12 and 1-15 of Seq ID No 330; 4-29, 31-42, 52-58 and 6-16 of Seq ID No 331; 4-9, 24-32 and 9-19 of Seq ID No 332; 4-12, 18-27 and 5-18 of Seq ID No 333; 4-11, 37-56, 58-92 and 18-29 of Seq ID No 334; 8-28 and 20-35 of Seq ID No 335; 4-15 of Seq ID No 336; 4-23, 27-39, 55-63 and 35-58 of Seq ID No 337; 6-26, 28-54 and 28-47 of Seq ID No 338; 4-10, 38-52, 58-82 and 30-49 of Seq ID No 339; 4-22, 29-35, 44-50, 53-68, 70-80 and 20-33 of Seq ID No 340; 22-28, 30-36 and 18-33 of Seq ID No 341; 4-11, 13-21, 25-30 and 20-30 of Seq ID No 342; 10-22 and 10-23 of Seq ID No 343; 4-11 and 9-20 of Seq ID No 344; 14-25, 32-46 and 6-19 of Seq ID No 345; 5-30 and 14-33 of Seq ID No 346; 4-15, 28-35, 46-55, 59-65, 76-84 and 9-24 of Seq ID No 347; 27-33 and 5-19 of Seq ID No 348; 5-13 and 8-18 of Seq ID No 349; 9-22, 24-34 and 21-40 of Seq ID No 350; 4-17, 35-41, 46-89, 93-98 and 71-89 of Seq ID No 351; 4-12, 14-24 and 2-17 of Seq ID No 352; 9-17 and 5-16 of Seq ID No 353; 7-41, 48-58, 63-75, 80-89 and 43-53 of Seq ID No 354; 4-22, 25-30 and 4-14 of Seq ID No 355; 4-55 and 18-33 of Seq ID No 356; 262-280 of Seq ID No 179; 131-146 of Seq ID No 186; 207-224 of Seq ID No 188; 27-50, 203-217 and 313-325 of Seq ID No 189; 110-129 of Seq ID No 192; 156-179, 174-197, 192-215, 210-233, 228-251 and 246-267 of Seq ID No 190; 377-400 of Seq ID No 196; 34-43, 234-257 and 350-367 of Seq ID No 203; 304-327 of Seq ID No 207; 25-48, 43-66 and 61-82 of Seq ID No 222; 398-421, 416-439, 434-457, 452-475, 470-493, 488-511, 506-529, 524-547, 621-644, 639-664, 707-730, 725-748, 743-766, 761-784, 779-802, 797-820, 984-1007, 1002-1025, 1020-1043, 1038-1061, 1056-1079, 1074-1097, 1092-1115, 1286-1309, 1304-1327, 1322-1345, 1340-1363, 1358-1381, 1376-1399, 1394-1417, 1412-1435, 1430-1453, 1448-1471, 1466-1489 and 1484-1507 of Seq ID No 226; 188-211, 206-229, 224-247, 242-265, 260-283 and 278-296 of Seq ID No 227; 56-79 and 122-132 of Seq ID No 229; 35-46 of Seq ID No 231; 178-201, 196-219, 214-237, 232-255, 250-273, 268-291, 379-402, 397-420, 415-438, 433-456, 451-474, 642-665, 660-683, 678-701, 696-719, 714-737, 732-755, 750-773, 768-791, 899-922, 917-940, 935-958, 1037-1060, 1055-1078, 1073-1096 and 1091-1114 of Seq ID No 232; 330-346 of Seq ID No 233; 571-594, 589-612, 607-630, 625-648, 643-666 and 661-684 of Seq ID No 242; 188-207 of Seq ID No 244; 61-84, 308-331, 326-349, 344-367, 362-385, 380-403 and 398-421 of Seq ID No 249; 79-98, 345-366, 844-867, 870-887 and 890-905 of Seq ID No 258; 94-109 of Seq ID No 268; 188-207 of Seq ID No 272; 290-306 of Seq ID No 276; 826-849 of Seq ID No 277; 228-252, 247-270, 265-288, 283-306, 301-324, 955-978, 973-996, 991-1014, 1009-1032, 1027-1050, 1045-1068, 2533-2556, 2551-2574, 2569-2592, 2587-2610, 2605-2628 and 2623-2646 of Seq ID No 279; 86-109 and 104-127 of Seq ID No 288; 546-560 of Seq ID No 289; 260-271 of Seq ID No 290; 106-129, 124-147, 142-165, 160-183, 178-201 and 375-398 of Seq ID No 291; 284-307 of Seq ID No 292; 362-385 of Seq ID No 301, and fragments comprising at least 6, preferably more than 8, especially more than 10 aa of said sequences. All these fragments individually and each independently form a preferred selected aspect of the present invention.

All linear hyperimmune serum reactive fragments of a particular antigen may be identified by analysing the entire sequence of the protein antigen by a set of peptides overlapping by 1 amino acid with a length of at least 10 amino acids. Subsequently, non-linear epitopes can be identified by analysis of the protein antigen with hyperimmune sera using the expressed full-length protein or domain polypeptides thereof. Assuming that a distinct domain of a protein is sufficient to form the 3D structure independent from the native protein, the analysis of the respective recombinant or synthetically produced domain polypeptide with hyperimmune serum would allow the identification of conformational epitopes within the individual domains of multi-domain proteins. For those antigens where a domain possesses linear as well as conformational epitopes, competition experiments with peptides corresponding to the linear epitopes may be used to confirm the presence of conformational epitopes.

It will be appreciated that the invention also relates to, among others, nucleic acid molecules encoding the aforementioned fragments, nucleic acid molecules that hybridise to nucleic acid molecules encoding the fragments, particularly those that hybridise under stringent conditions, and nucleic acid molecules, such as PCR primers, for amplifying nucleic acid molecules that encode the fragments. In these regards, preferred nucleic acid molecules are those that correspond to the preferred fragments, as discussed above.

The present invention also relates to vectors, which comprise a nucleic acid molecule or nucleic acid molecules of the present invention, host cells which are genetically engineered with vectors of the invention and the production of hyperimmune serum reactive antigens and fragments thereof by recombinant techniques.

A great variety of expression vectors can be used to express a hyperimmune serum reactive antigen or fragment thereof according to the present invention. Generally, any vector suitable to maintain, propagate or express nucleic acids to express a polypeptide in a host may be used for expression in this regard. In accordance with this aspect of the invention the vector may be, for example, a plasmid vector, a single or double-stranded phage vector, a single or double-stranded RNA or DNA viral vector. Starting plasmids disclosed herein are either commercially available, publicly available, or can be constructed from available plasmids by routine application of well-known, published procedures. Preferred among vectors, in certain respects, are those for expression of nucleic acid molecules and hyperimmune serum reactive antigens or fragments thereof of the present invention. Nucleic acid constructs in host cells can be used in a conventional manner to produce the gene product encoded by the recombinant sequence. Alternatively, the hyperimmune serum reactive antigens and fragments thereof of the invention can be synthetically produced by conventional peptide synthesizers. Mature proteins can be expressed in mammalian cells, yeast, bacteria, or other cells under the control of appropriate promoters. Cell-free translation systems can also be employed to produce such proteins using RNAs derived from the DNA construct of the present invention.

Host cells can be genetically engineered to incorporate nucleic acid molecules and express nucleic acid molecules of the present invention. Representative examples of appropriate hosts include bacterial cells, such as streptococci, staphylococci, E. coli, Streptomyces and Bacillus subtillis cells; fungal cells, such as yeast cells and Aspergillus cells; insect cells such as Drosophila S2 and Spodoptera Sf9 cells; animal cells such as CHO, COS, Hela, C127, 3T; 3, BHK, 293 and Bowes melanoma cells; and plant cells.

The invention also provides a process for producing a H. pylori hyperimmune serum reactive antigen and a fragment thereof comprising expressing from the host cell a hyperimmune serum reactive antigen or fragment thereof encoded by the nucleic acid molecules provided by the present invention. The invention further provides a process for producing a cell, which expresses a H. pylori hyperimmune serum reactive antigen or a fragment thereof comprising transforming or transfecting a suitable host cell with the vector according to the present invention such that the transformed or transfected cell expresses the polypeptide encoded by the nucleic acid contained in the vector.

The polypeptide may be expressed in a modified form, such as a fusion protein, and may include not only secretion signals but also additional heterologous functional regions. Thus, for instance, a region of additional amino acids, particularly charged amino acids, may be added to the N- or C-terminus of the polypeptide to improve stability and persistence in the host cell, during purification or during subsequent handling and storage. Also, regions may be added to the polypeptide to facilitate purification. Such regions may be removed prior to final preparation of the polypeptide. The addition of peptide moieties to polypeptides to engender secretion or excretion, to improve stability or to facilitate, purification, among others, are familiar and routine techniques in the art. A preferred fusion protein comprises a heterologous region from immunoglobulin that is useful to solubilize or purify polypeptides. For example, EP-A-O 464 533 (Canadian counterpart 2045869) discloses fusion proteins comprising various portions of constant region of immunoglobin molecules together with another protein or part thereof. In drug discovery, for example, proteins have been fused with antibody Fc portions for the purpose of high-throughout screening assays to identify antagonists. See for example, {Bennett, D. et al., 1995} and {Johanson, K et al., 1995}.

The H. pylori hyperimmune serum reactive antigen or a fragment thereof can be recovered and purified from recombinant cell cultures by well-known methods including ammonium sulfate or ethanol precipitation, acid extraction, anion or cation exchange chromatography, phosphocellulose chromatography, hydrophobic interaction chromatography, hydroxylapatite chromatography and lectin chromatography.

The hyperimmune serum reactive antigens and fragments thereof according to the present invention can be produced by chemical synthesis as well as by biotechnological means. The latter comprise the transfection or transformation of a host cell with a vector containing a nucleic acid according to the present invention and the cultivation of the transfected or transformed host cell under conditions, which are known to the ones skilled in the art. The production method may also comprise a purification step in order to purify or isolate the polypeptide to be manufactured. In a preferred embodiment the vector is a vector according to the present invention.

The hyperimmune serum reactive antigens and fragments thereof according to the present invention may be used for the detection of the organism or organisms in a sample containing these organisms or polypeptides derived thereof. Preferably such detection is for diagnosis, more preferable for the diagnosis of a disease, most preferably for the diagnosis of diseases related or linked to the presence or abundance of Gram-negative bacteria, especially bacteria selected from the group comprising Helicobacter, Campylobacter and Arcobacter. More preferably, the microorganisms are selected from the group comprising Helicobacter cinaedi and Helicobacter fanneliae, especially the microorganism is Helicobacter pylori.

The present invention also relates to diagnostic assays such as quantitative and diagnostic assays for detecting levels of the hyperimmune serum reactive antigens and fragments thereof of the present invention in cells and tissues, including determination of normal and abnormal levels. Thus, for instance, a diagnostic assay in accordance with the invention for detecting over-expression of the polypeptide compared to normal control tissue samples may be used to detect the presence of an infection, for example, and to identify the infecting organism. Assay techniques that can be used to determine levels of a polypeptide, in a sample derived from a host are well-known to those of skill in the art. Such assay methods include radioimmunoassays, competitive-binding assays, Western Blot analysis and ELISA assays. Among these, ELISAs frequently are preferred. An ELISA assay initially comprises preparing an antibody specific to the polypeptide, preferably a monoclonal antibody. In addition, a reporter antibody generally is prepared which binds to the monoclonal antibody. The reporter antibody is attached to a detectable reagent such as radioactive, fluorescent or enzymatic reagent, such as horseradish peroxidase enzyme.

The hyperimmune serum reactive antigens and fragments thereof according to the present invention may also be used for the purpose of or in connection with an array. More particularly, at least one of the hyperimmune serum reactive antigens and fragments thereof according to the present invention may be immobilized on a support. Said support typically comprises a variety of hyperimmune serum reactive antigens and fragments thereof whereby the variety may be created by using one or several of the hyperimmune serum reactive antigens and fragments thereof according to the present invention and/or hyperimmune serum reactive antigens and fragments thereof being different. The characterizing feature of such array as well as of any array in general is the fact that at a distinct or predefined region or position on said support or a surface thereof, a distinct polypeptide is immobilized. Because of this any activity at a distinct position or region of an array can be correlated with a specific polypeptide. The number of different hyperimmune serum reactive antigens and fragments thereof immobilized on a support may range from as little as 10 to several 1000 different hyperimmune serum reactive antigens and fragments thereof. The density of hyperimmune serum reactive antigens and fragments thereof per cm² is in a preferred embodiment as little as 10 peptides/polypeptides per cm² to at least 400 different peptides/polypeptides per cm² and more particularly at least 1000 different hyperimmune serum reactive antigens and fragments thereof per cm².

The manufacture of such arrays is known to the one skilled in the art and, for example, described in U.S. Pat. No. 5,744,309. The array preferably comprises a planar, porous or non-porous solid support having at least a first surface. The hyperimmune serum reactive antigens and fragments thereof as disclosed herein, are immobilized on said surface. Preferred support materials are, among others, glass or cellulose. It is also within the present invention that the array is used for any of the diagnostic applications described herein. Apart from the hyperimmune serum reactive antigens and fragments thereof according to the present invention also the nucleic acid molecules according to the present invention may be used for the generation of an array as described above. This applies as well to an array made of antibodies, preferably monoclonal antibodies as, among others, described herein.

In a further aspect the present invention relates to an antibody directed to any of the hyperimmune serum reactive antigens and fragments thereof, derivatives or fragments thereof according to the present invention. The present invention includes, for example, monoclonal and polyclonal antibodies, chimeric, single chain, and humanized antibodies, as well as Fab fragments, or the product of a Fab expression library. It is within the present invention that the antibody may be chimeric, i.e. that different parts thereof stem from different species or at least the respective sequences are taken from different species.

Antibodies generated against the hyperimmune serum reactive antigens and fragments thereof corresponding to a sequence of the present invention can be obtained by direct injection of the hyperimmune serum reactive antigens and fragments thereof into an animal or by administering the hyperimmune serum reactive antigens and fragments thereof to an animal, preferably a non-human. The antibody so obtained will then bind the hyperimmune serum reactive antigens and fragments thereof itself. In this manner, even a sequence encoding only a fragment of a hyperimmune serum reactive antigen and fragments thereof can be used to generate antibodies binding the whole native hyperimmune serum reactive antigen and fragments thereof. Such antibodies can then be used to isolate the hyperimmune serum reactive antigens and fragments thereof from tissue expressing those hyperimmune serum reactive antigens and fragments thereof.

For preparation of monoclonal antibodies, any technique known in the art, which provides antibodies produced by continuous cell line cultures can be used. (as described originally in {Kohler, G. et al., 1975}.

Techniques described for the production of single chain antibodies (U.S. Pat. No. 4,946,778) can be adapted to produce single chain antibodies to immunogenic hyperimmune serum reactive antigens and fragments thereof according to this invention. Also, transgenic mice, or other organisms such as other mammals, may be used to express humanized antibodies to immunogenic hyperimmune serum reactive antigens and fragments thereof according to this invention.

Alternatively, phage display technology or ribosomal display could be utilized to select antibody genes with binding activities towards the hyperimmune serum reactive antigens and fragments thereof either from repertoires of PCR amplified v-genes of lymphocytes from humans screened for possessing respective target antigens or from naïve libraries {McCafferty, J. et al., 1990}; {Marks, J. et al., 1992}. The affinity of these antibodies can also be improved by chain shuffling {Clackson, T. et al., 1991}.

If two antigen binding domains are present, each domain may be directed against a different epitope—termed ‘bispecific’ antibodies.

The above-described antibodies may be employed to isolate or to identify clones expressing the hyperimmune serum reactive antigens and fragments thereof or purify the hyperimmune serum reactive antigens and fragments thereof of the present invention by attachment of the antibody to a solid support for isolation and/or purification by affinity chromatography.

Thus, among others, antibodies against the hyperimmune serum reactive antigens and fragments thereof of the present invention may be employed to inhibit and/or treat infections, particularly bacterial infections and especially infections arising from H. pylori.

Hyperimmune serum reactive antigens and fragments thereof include antigenically, epitopically or immunologically equivalent derivatives, which form a particular aspect of this invention. The term “antigenically equivalent derivative” as used herein encompasses a hyperimmune serum reactive antigen and fragments thereof or its equivalent which will be specifically recognized by certain antibodies which, when raised to the protein or hyperimmune serum reactive antigen and fragments thereof according to the present invention, interfere with the interaction between pathogen and mammalian host. The term “immunologically equivalent derivative” as used herein encompasses a peptide or its equivalent which when used in a suitable formulation to raise antibodies in a vertebrate, the antibodies act to interfere with the interaction between pathogen and mammalian host.

The hyperimmune serum reactive antigens and fragments thereof, such as an antigenically or immunologically equivalent derivative or a fusion protein thereof can be used as an antigen to immunize a mouse or other animal such as a rat or chicken. The fusion protein may provide stability to the hyperimmune serum reactive antigens and fragments thereof. The antigen may be associated, for example by conjugation, with an immunogenic carrier protein, for example bovine serum albumin (BSA) or keyhole limpet haemocyanin (KLH). Alternatively, an antigenic peptide comprising multiple copies of the protein or hyperimmune serum reactive antigen and fragments thereof, or an antigenically or immunologically equivalent hyperimmune serum reactive antigen and fragments thereof, may be sufficiently antigenic to improve immunogenicity so as to obviate the use of a carrier.

Preferably the antibody or derivative thereof is modified to make it less immunogenic in the individual. For example, if the individual is human the antibody may most preferably be “humanized”, wherein the complimentarity determining region(s) of the hybridoma-derived antibody has been transplanted into a human monoclonal antibody, for example as described in {Jones, P. et al., 1986} or {Tempest, P. et al., 1991}.

The use of a polynucleotide of the invention in genetic immunization will preferably employ a suitable delivery method such as direct injection of plasmid DNA into muscle, delivery of DNA complexed with specific protein carriers, coprecipitation of DNA with calcium phosphate, encapsulation of DNA in various forms of liposomes, particle bombardment {Tang D. et al., 1992}, {Eisenbraun, M. et al., 1993} and in vivo infection using cloned retroviral vectors {Seeger, C. et al., 1984}.

In a further aspect the present invention relates to a peptide binding to any of the hyperimmune serum reactive antigens and fragments thereof according to the present invention, and a method for the manufacture of such peptides whereby the method is characterized by the use of the hyperimmune serum reactive antigens and fragments thereof according to the present invention and the basic steps are known to the one skilled in the art.

Such peptides may be generated by using methods according to the state of the art such as phage display or ribosome display. In case of phage display, basically a library of peptides is generated, in form of phages, and this kind of library is contacted with the target molecule, in the present case a hyperimmune serum reactive antigen and fragments thereof according to the present invention. Those peptides binding to the target molecule are subsequently removed, preferably as a complex with the target molecule, from the respective reaction. It is known to the one skilled in the art that the binding characteristics, at least to a certain extent, depend on the particularly realized experimental set-up such as the salt concentration and the like. After separating those peptides binding to the target molecule with a higher affinity or a bigger force, from the non-binding members of the library, and optionally also after removal of the target molecule from the complex of target molecule and peptide, the respective peptide(s) may subsequently be characterised. Prior to the characterisation optionally an amplification step is realized such as, e.g. by propagating the peptide encoding phages. The characterisation preferably comprises the sequencing of the target binding peptides. Basically, the peptides are not limited in their lengths, however, preferably peptides having a lengths from about 8 to 20 amino acids are preferably obtained in the respective methods. The size of the libraries may be about 10² to 10¹⁸, preferably 10⁸ to 10¹⁵ different peptides, however, is not limited thereto.

A particular form of target binding hyperimmune serum reactive antigens and fragments thereof are the so-called “anticalines” which are, among others, described in the German patent application DE 197 42 706.

In a further aspect the present invention relates to functional nucleic acids interacting with any of the hyperimmune serum reactive antigens and fragments thereof according to the present invention, and a method for the manufacture of such functional nucleic acids whereby the method is characterized by the use of the hyperimmune serum reactive antigens and fragments thereof according to the present invention and the basic steps are known to the one skilled in the art. The functional nucleic acids are preferably aptamers and spiegelmers.

Aptamers are D-nucleic acids, which are either single stranded or double stranded and which specifically interact with a target molecule. The manufacture or selection of aptamers is, e.g., described in European patent EP 0 533 838. Basically the following steps are realized. First, a mixture of nucleic acids, i.e. potential aptamers, is provided whereby each nucleic acid typically comprises a segment of several, preferably at least eight subsequent randomised nucleotides. This mixture is subsequently contacted with the target molecule whereby the nucleic acid(s) bind to the target molecule, such as based on an increased affinity towards the target or with a bigger force thereto, compared to the candidate mixture. The binding nucleic acid(s) are/is subsequently separated from the remainder of the mixture. Optionally, the thus obtained nucleic acid(s) is amplified using, e.g. polymerase chain reaction. These steps may be repeated several times giving at the end a mixture having an increased ratio of nucleic acids specifically binding to the target from which the final binding nucleic acid is then optionally selected. These specifically binding nucleic acid(s) are referred to as aptamers. It is obvious that at any stage of the method for the generation or identification of the aptamers samples of the mixture of individual nucleic acids may be taken to determine the sequence thereof using standard techniques. It is within the present invention that the aptamers may be stabilized such as, e.g., by introducing defined chemical groups which are known to the one skilled in the art of generating aptamers. Such modification may for example reside in the introduction of an amino group at the 2′-position of the sugar moiety of the nucleotides. Aptamers are currently used as therapeutical agents. However, it is also within the present invention that the thus selected or generated aptamers may be used for target validation and/or as lead substance for the development of medicaments, preferably of medicaments based on small molecules. This is actually done by a competition assay whereby the specific interaction between the target molecule and the aptamer is inhibited by a candidate drug whereby upon replacement of the aptamer from the complex of target and aptamer it may be assumed that the respective drug candidate allows a specific inhibition of the interaction between target and aptamer, and if the interaction is specific, said candidate drug will, at least in principle, be suitable to block the target and thus decrease its biological availability or activity in a respective system comprising such target. The thus obtained small molecule may then be subject to further derivatisation and modification to optimise its physical, chemical, biological and/or medical characteristics such as toxicity, specificity, biodegradability and bioavailability.

Spiegelmers and their generation or manufacture is based on a similar principle. The manufacture of spiegelmers is described in international patent application WO 98/08856. Spiegelmers are L-nucleic acids, which means that they are composed of L-nucleotides rather than D-nucleotides as aptamers are. Spiegelmers are characterized by the fact that they have a very high stability in biological systems and, comparable to aptamers, specifically interact with the target molecule against which they are directed. In the process of generating spiegelmers, a heterogeonous population of D-nucleic acids is created and this population is contacted with the optical antipode of the target molecule, in the present case for example with the D-enantiomer of the naturally occurring L-enantiomer of the hyperimmune serum reactive antigens and fragments thereof according to the present invention. Subsequently, those D-nucleic acids are separated which do not interact with the optical antipode of the target molecule. But those D-nucleic acids interacting with the optical antipode of the target molecule are separated, optionally identified and/or sequenced and subsequently the corresponding L-nucleic acids are synthesized based on the nucleic acid sequence information obtained from the D-nucleic acids. These L-nucleic acids which are identical in terms of sequence with the aforementioned D-nucleic acids interacting with the optical antipode of the target molecule, will specifically interact with the naturally occurring target molecule rather than with the optical antipode thereof. Similar to the method for the generation of aptamers it is also possible to repeat the various steps several times and thus to enrich those nucleic acids specifically interacting with the optical antipode of the target molecule.

In a further aspect the present invention relates to functional nucleic acids interacting with any of the nucleic acid molecules according to the present invention, and a method for the manufacture of such functional nucleic acids whereby the method is characterized by the use of the nucleic acid molecules and their respective sequences according to the present invention and the basic steps are known to the one skilled in the art. The functional nucleic acids are preferably ribozymes, antisense oligonucleotides and siRNA.

Ribozymes are catalytically active nucleic acids, which preferably consist of RNA which basically comprises two moieties. The first moiety shows a catalytic activity whereas the second moiety is responsible for the specific interaction with the target nucleic acid, in the present case the nucleic acid coding for the hyperimmune serum reactive antigens and fragments thereof according to the present invention. Upon interaction between the target nucleic acid and the second moiety of the ribozyme, typically by hybridisation and Watson-Crick base pairing of essentially complementary stretches of bases on the two hybridising strands, the catalytically active moiety may become active which means that it catalyses, either intramolecularly or intermolecularly, the target nucleic acid in case the catalytic activity of the ribozyme is a phosphodiesterase activity. Subsequently, there may be a further degradation of the target nucleic acid, which in the end results in the degradation of the target nucleic acid as well as the protein derived from the said target nucleic acid. Ribozymes, their use and design principles are known to the one skilled in the art, and, for example described in {Doherty, E. et al., 2001} and {Lewin, A. et al., 2001}.

The activity and design of antisense oligonucleotides for the manufacture of a medicament and as a diagnostic agent, respectively, is based on a similar mode of action. Basically, antisense oligonucleotides hybridise based on base complementarity, with a target RNA, preferably with a mRNA, thereby activating RNase H. RNase H is activated by both phosphodiester and phosphorothioate-coupled DNA. Phosphodiester-coupled DNA, however, is rapidly degraded by cellular nucleases with the exception of phosphorothioate-coupled DNA. These resistant, non-naturally occurring DNA derivatives do not inhibit RNase H upon hybridisation with RNA. In other words, antisense polynucleotides are only effective as DNA RNA hybride complexes. Examples for this kind of antisense oligonucleotides are described, among others, in US-patent U.S. Pat. No. 5,849,902 and U.S. Pat. No. 5,989,912. In other words, based on the nucleic acid sequence of the target molecule which in the present case are the nucleic acid molecules for the hyperimmune serum reactive antigens and fragments thereof according to the present invention, either from the target protein from which a respective nucleic acid sequence may in principle be deduced, or by knowing the nucleic acid sequence as such, particularly the mRNA, suitable antisense oligonucleotides may be designed base on the principle of base complementarity.

Particularly preferred are antisense-oligonucleotides, which have a short stretch of phosphorothioate DNA (3 to 9 bases). A minimum of 3 DNA bases is required for activation of bacterial RNase H and a minimum of 5 bases is required for mammalian RNase H activation. In these chimeric oligonucleotides there is a central region that forms a substrate for RNase H that is flanked by hybridising “arms” comprised of modified nucleotides that do not form substrates for RNase H. The hybridising arms of the chimeric oligonucleotides may be modified such as by 2′-O-methyl or 2′-fluoro. Alternative approaches used methylphosphonate or phosphoramidate linkages in said arms. Further embodiments of the antisense oligonucleotide useful in the practice of the present invention are P-methoxyoligonucleotides, partial P-methoxyoligodeoxyribonucleotides or P-methoxyoligonucleotides.

Of particular relevance and usefulness for the present invention are those antisense oligonucleotides as more particularly described in the above two mentioned US patents. These oligonucleotides contain no naturally occurring 5′→3′-linked nucleotides. Rather the oligonucleotides have two types of nucleotides: 2′-deoxyphosphorothioate, which activate RNase H, and 2′-modified nucleotides, which do not. The linkages between the 2′-modified nucleotides can be phosphodiesters, phosphorothioate or P-ethoxyphosphodiester. Activation of RNase H is accomplished by a contiguous RNase H-activating region, which contains between 3 and 5 2′-deoxyphosphorothioate nucleotides to activate bacterial RNase H and between 5 and 10 2′-deoxyphosphorothioate nucleotides to activate eucaryotic and, particularly, mammalian RNase H. Protection from degradation is accomplished by making the 5′ and 3′ terminal bases highly nuclease resistant and, optionally, by placing a 3′ terminal blocking group.

More particularly, the antisense oligonucleotide comprises a 5′ terminus and a 3′ terminus; and from position 11 to 59 5′→3′-linked nucleotides independently selected from the group consisting of 2′-modified phosphodiester nucleotides and 2′-modified P-alkyloxyphosphotriester nucleotides; and wherein the 5′-terminal nucleoside is attached to an RNase H-activating region of between three and ten contiguous phosphorothioate-linked deoxyribonucleotides, and wherein the 3′-terminus of said oligonucleotide is selected from the group consisting of an inverted deoxyribonucleotide, a contiguous stretch of one to three phosphorothioate 2′-modified ribonucleotides, a biotin group and a P-alkyloxyphosphotriester nucleotide.

Also an antisense oligonucleotide may be used wherein not the 5′ terminal nucleoside is attached to an RNase H-activating region but the 3′ terminal nucleoside as specified above. Also, the 5′ terminus is selected from the particular group rather than the 3′ terminus of said oligonucleotide.

The nucleic acids as well as the hyperimmune serum reactive antigens and fragments thereof according to the present invention may be used as or for the manufacture of pharmaceutical compositions, especially vaccines. Preferably such pharmaceutical composition, preferably vaccine is for the prevention or treatment of diseases caused by, related to or associated with H. pylori. In so far another aspect of the invention relates to a method for inducing an immunological response in an individual, particularly a mammal, which comprises inoculating the individual with the hyperimmune serum reactive antigens and fragments thereof of the invention, or a fragment or variant thereof, adequate to produce antibodies to protect said individual from infection, particularly Helicobacter infection and most particularly H. pylori infections.

Yet another aspect of the invention relates to a method of inducing an immunological response in an individual which comprises, through gene therapy or otherwise, delivering a nucleic acid functionally encoding hyperimmune serum reactive antigens and fragments thereof, or a fragment or a variant thereof, for expressing the hyperimmune serum reactive antigens and fragments thereof, or a fragment or a variant thereof in vivo in order to induce an immunological response to produce antibodies or a cell mediated T cell response, either cytokine-producing T cells or cytotoxic T cells, to protect said individual from disease, whether that disease is already established within the individual or not. One way of administering the gene is by accelerating it into the desired cells as a coating on particles or otherwise.

A further aspect of the invention relates to an immunological composition which, when introduced into a host capable of having induced within it an immunological response, induces an immunological response in such host, wherein the composition comprises recombinant DNA which codes for and expresses an antigen of the hyperimmune serum reactive antigens and fragments thereof of the present invention. The immunological response may be used therapeutically or prophylactically and may take the form of antibody immunity or cellular immunity such as that arising from CTL or CD4+ T cells.

The hyperimmune serum reactive antigens and fragments thereof of the invention or a fragment thereof may be fused with a co-protein which may not by itself produce antibodies, but is capable of stabilizing the first protein and producing a fused protein which will have immunogenic and protective properties. This fused recombinant protein preferably further comprises an antigenic co-protein, such as Glutathione-S-transferase (GST) or beta-galactosidase, relatively large co-proteins which solubilise the protein and facilitate production and purification thereof. Moreover, the co-protein may act as an adjuvant in the sense of providing a generalized stimulation of the immune system. The co-protein may be attached to either the amino or carboxy terminus of the first protein.

Also, provided by this invention are methods using the described nucleic acid molecule or particular fragments thereof in such genetic immunization experiments in animal models of infection with H. pylori. Such fragments will be particularly useful for identifying protein epitopes able to provoke a prophylactic or therapeutic immune response. This approach can allow for the subsequent preparation of monoclonal antibodies of particular value from the requisite organ of the animal successfully resisting or clearing infection for the development of prophylactic agents or therapeutic treatments of H. pylori infection in mammals, particularly humans.

The hyperimmune serum reactive antigens and fragments thereof may be used as an antigen for vaccination of a host to produce specific antibodies which protect against invasion of bacteria, for example by blocking adherence of bacteria to damaged tissue. Examples of tissue damage include wounds in skin or connective tissue caused e.g. by mechanical, chemical or thermal damage or by implantation of indwelling devices, or wounds in the mucous membranes, such as the mouth, mammary glands, urethra or vagina.

The present invention also includes a vaccine formulation, which comprises the immunogenic recombinant protein together with a suitable carrier. Since the protein may be broken down in the stomach, it is preferably administered parenterally, including, for example, administration that is subcutaneous, intramuscular, intravenous, intradermal intranasal or transdermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampoules and vials, and may be stored in a freeze-dried condition requiring only the addition of the sterile liquid carrier immediately prior to use. The vaccine formulation may also include adjuvant systems for enhancing the immunogenicity of the formulation, such as oil-in-water systems and other systems known in the art. The dosage will depend on the specific activity of the vaccine and can be readily determined by routine experimentation.

According to another aspect, the present invention relates to a pharmaceutical composition comprising such a hyperimmune serum-reactive antigen or a fragment thereof as provided in the present invention for H. pylori. Such a pharmaceutical composition may comprise one, preferably at least two or more hyperimmune serum reactive antigens or fragments thereof against H. pylori. Optionally, such H. pylori hyperimmune serum reactive antigens or fragments thereof may also be combined with antigens against other pathogens in a combination pharmaceutical composition. Preferably, said pharmaceutical composition is a vaccine for preventing or treating an infection caused by H. pylori and/or other pathogens against which the antigens have been included in the vaccine.

According to a further aspect, the present invention relates to a pharmaceutical composition comprising a nucleic acid molecule encoding a hyperimmune serum-reactive antigen or a fragment thereof as identified above for H. pylori Such a pharmaceutical composition may comprise one or more nucleic acid molecules encoding hyperimmune serum reactive antigens or fragments thereof against H. pylori. Optionally, such H. pylori nucleic acid molecules encoding hyperimmune serum reactive antigens or fragments thereof may also be combined with nucleic acid molecules encoding antigens against other pathogens in a combination pharmaceutical composition. Preferably, said pharmaceutical composition is a vaccine for preventing or treating an infection caused by H. pylori and/or other pathogens against which the antigens have been included in the vaccine.

The pharmaceutical composition may contain any suitable auxiliary substances, such as buffer substances, stabilisers or further active ingredients, especially ingredients known in connection of pharmaceutical composition and/or vaccine production.

A preferable carrier/or excipient for the hyperimmune serum-reactive antigens, fragments thereof or a coding nucleic acid molecule thereof according to the present invention is an immunostimulatory compound for further stimulating the immune response to the given hyperimmune serum-reactive antigen, fragment thereof or a coding nucleic acid molecule thereof. Preferably the immunostimulatory compound in the pharmaceutical preparation according to the present invention is selected from the group of polycationic substances, especially polycationic peptides, immunostimulatory nucleic acids molecules, preferably immunostimulatory deoxynucleotides, alum, Freund's complete adjuvants, Freund's incomplete adjuvants, neuroactive compounds, especially human growth hormone, or combinations thereof.

It is also within the scope of the present invention that the pharmaceutical composition, especially vaccine, comprises apart from the hyperimmune serum reactive antigens, fragments thereof and/or coding nucleic acid molecules thereof according to the present invention other compounds which are biologically or pharmaceutically active. Preferably, the vaccine composition comprises at least one polycationic peptide. The polycationic compound(s) to be used according to the present invention may be any polycationic compound, which shows the characteristic effects according to the WO 97/30721. Preferred polycationic compounds are selected from basic polyppetides, organic polycations, basic polyamino acids or mixtures thereof. These polyamino acids should have a chain length of at least 4 amino acid residues (WO 97/30721). Especially preferred are substances like polylysine, polyarginine and polypeptides containing more than 20%, especially more than 50% of basic amino acids in a range of more than 8, especially more than 20, amino acid residues or mixtures thereof. Other preferred polycations and their pharmaceutical compositions are described in WO 97/30721 (e.g. polyethyleneimine) and WO 99/38528. Preferably these polypeptides contain between 20 and 500 amino acid residues, especially between 30 and 200 residues.

These polycationic compounds may be produced chemically or recombinantly or may be derived from natural sources.

Cationic (poly)peptides may also be anti-microbial with properties as reviewed in {Ganz, T., 1999}. These (poly)peptides may be of prokaryotic or animal or plant origin or may be produced chemically or recombinantly (WO 02/13857). Peptides may also belong to the class of defensins (WO 02/13857). Sequences of such peptides can be, for example, found in the Antimicrobial Sequences Database under the following internet address:
http://www.bbcm.univ.trieste.it/˜tossi/pag2.html

Such host defence peptides or defensives are also a preferred form of the polycationic polymer according to the present invention. Generally, a compound allowing as an end product activation (or down-regulation) of the adaptive immune system, preferably mediated by APCs (including dendritic cells) is used as polycationic polymer.

Especially preferred for use as polycationic substances in the present invention are cathelicidin derived antimicrobial peptides or derivatives thereof (International patent application WO 02/13857, incorporated herein by reference), especially antimicrobial peptides derived from mammalian cathelicidin, preferably from human, bovine or mouse.

Polycationic compounds derived from natural sources include HIV-REV or HIV-TAT (derived cationic peptides, antennapedia peptides, chitosan or other derivatives of chitin) or other peptides derived from these peptides or proteins by biochemical or recombinant production. Other preferred polycationic compounds are cathelin or related or derived substances from cathelin. For example, mouse cathelin is a peptide, which has the amino acid sequence NH₂-RLAGLLRKGGEKIGEKLKKIGOKIKNFFQKLVPQPE-COOH. Related or derived cathelin substances contain the whole or parts of the cathelin sequence with at least 15-20 amino acid residues. Derivations may include the substitution or modification of the natural amino acids by amino acids, which are not among the 20 standard amino acids. Moreover, further cationic residues may be introduced into such cathelin molecules. These cathelin molecules are preferred to be combined with the antigen. These cathelin molecules surprisingly have turned out to be also effective as an adjuvant for an antigen without the addition of further adjuvants. It is therefore possible to use such cathelin molecules as efficient adjuvants in vaccine formulations with or without further immunactivating substances.

Another preferred polycationic substance to be used according to the present invention is a synthetic peptide containing at least 2 KLK-motifs separated by a linker of 3 to 7 hydrophobic amino acids (International patent application WO 02/32451, incorporated herein by reference).

The pharmaceutical composition of the present invention may further comprise immunostimulatory nucleic acid(s). Immunostimulatory nucleic acids are e.g. neutral or artificial CpG containing nucleic acids, short stretches of nucleic acids derived from non-vertebrates or in form of short oligonucleotides (ODNs) containing non-methylated cytosine-guanine di-nucleotides (CpG) in a certain base context (e.g. described in WO 96/02555). Alternatively, also nucleic acids based on inosine and cytidine as e.g. described in the WO 01/93903, or deoxynucleic acids containing deoxy-inosine and/or deoxyuridine residues (described in WO 01/93905 and PCT/EP 02/05448, incorporated herein by reference) may preferably be used as immunostimulatory nucleic acids for the present invention. Preferablly, the mixtures of different immunostimulatory nucleic acids may be used according to the present invention.

It is also within the present invention that any of the aforementioned polycationic compounds is combined with any of the immunostimulatory nucleic acids as aforementioned. Preferably, such combinations are according to the ones as described in WO 01/93905, WO 02/32451, WO 01/54720, WO 01/93903, WO 02/13857 and PCT/EP 02/05448 and the Austrian patent application A 1924/2001, incorporated herein by reference.

In addition or alternatively such vaccine composition may comprise apart from the hyperimmune serum reactive antigens and fragments thereof, and the coding nucleic acid molecules thereof according to the present invention a neuroactive compound. Preferably, the neuroactive compound is human growth factor as, e.g. described in WO 01/24822. Also preferably, the neuroactive compound is combined with any of the polycationic compounds and/or immunostimulatory nucleic acids as afore-mentioned.

In a further aspect the present invention is related to a pharmaceutical composition. Such pharmaceutical composition is, for example, the vaccine described herein. Also a pharmaceutical composition is a pharmaceutical composition which comprises any of the following compounds or combinations thereof: the nucleic acid molecules according to the present invention, the hyperimmune serum reactive antigens and fragments thereof according to the present invention, the vector according to the present invention, the cells according to the present invention, the antibody according to the present invention, the functional nucleic acids according to the present invention and the binding peptides such as the anticalines according to the present invention, any agonists and antagonists screened as described herein. In connection therewith any of these compounds may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of a hyperimmune serum reactive antigen and fragments thereof of the invention and a pharmaceutically acceptable carrier or excipient. Such carriers may include, but are not limited to, saline, buffered saline, dextrose, water, glycerol, ethanol and combinations thereof. The formulation should suit the mode of administration.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

In therapy or as a prophylactic, the active agent may be administered to an individual as an injectable composition, for example as a sterile aqueous dispersion, preferably isotonic.

Alternatively the composition may be formulated for topical application, for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

In addition to the therapy described above, the compositions of this invention may be used generally as a wound treatment agent to prevent adhesion of bacteria to matrix proteins exposed in wound tissue and for prophylactic use in dental treatment as an alternative to, or in conjunction with, antibiotic prophylaxis.

A vaccine composition is conveniently in injectable form. Conventional adjuvants may be employed to enhance the immune response. A suitable unit dose for vaccination is 0.05-5 μg antigen/per kg of body weight, and such dose is preferably administered 1-3 times and with an interval of 1-3 weeks.

With the indicated dose range, no adverse toxicological effects should be observed with the compounds of the invention, which would preclude their administration to suitable individuals.

In a further embodiment the present invention relates to diagnostic and pharmaceutical packs and kits comprising one or more containers filled with one or more of the ingredients of the aforementioned compositions of the invention. The ingredient(s) can be present in a useful amount, dosage, formulation or combination. Associated with such container(s) can be a notice in the form prescribed by a governmental agency regulating the manufacture, use or sale of pharmaceuticals or biological products, reflecting approval by the agency of the manufacture, use or sale of the product for human administration.

In connection with the present invention any disease related use as disclosed herein such as, e.g. use of the pharmaceutical composition or vaccine, is particularly a disease or diseased condition which is caused by, linked or associated with Helicobacter, more preferably, H. pylori. In connection therewith it is to be noted that H. pylori comprises several strains including those disclosed herein. A disease related, caused or associated with the bacterial infection to be prevented and/or treated according to the present invention includes besides others peptic ulcer and associated cancer in humans.

In a still further embodiment the present invention is related to a screening method using any of the hyperimmune serum reactive antigens or nucleic acids according to the present invention. Screening methods as such are known to the one skilled in the art and can be designed such that an agonist or an antagonist is screened. Preferably an antagonist is screened which in the present case inhibits or prevents the binding of any hyperimmune serum reactive antigen and fragment thereof according to the present invention to an interaction partner. Such interaction partner can be a naturally occurring interaction partner or a non-naturally occurring interaction partner.

The invention also provides a method of screening compounds to identify those, which enhance (agonist) or block (antagonist) the function of hyperimmune serum reactive antigens and fragments thereof or nucleic acid molecules of the present invention, such as its interaction with a binding molecule. The method of screening may involve high-throughput.

For example, to screen for agonists or antagonists, the interaction partner of the nucleic acid molecule and nucleic acid, respectively, according to the present invention, maybe a synthetic reaction mix, a cellular compartment, such as a membrane, cell envelope or cell wall, or a preparation of any thereof, may be prepared from a cell that expresses a molecule that binds to the hyperimmune serum reactive antigens and fragments thereof of the present invention. The preparation is incubated with labelled hyperimmune serum reactive antigens and fragments thereof in the absence or the presence of a candidate molecule, which may be an agonist or antagonist. The ability of the candidate molecule to bind the binding molecule is reflected in decreased binding of the labelled ligand. Molecules which bind gratuitously, i.e., without inducing the functional effects of the hyperimmune serum reactive antigens and fragments thereof, are most likely to be good antagonists. Molecules that bind well and elicit functional effects that are the same as or closely related to the hyperimmune serum reactive antigens and fragments thereof are good agonists.

The functional effects of potential agonists and antagonists may be measured, for instance, by determining the activity of a reporter system following interaction of the candidate molecule with a cell or appropriate cell preparation, and comparing the effect with that of the hyperimmune serum reactive antigens and fragments thereof of the present invention or molecules that elicit the same effects as the hyperimmune serum reactive antigens and fragments thereof. Reporter systems that may be useful in this regard include but are not limited to colorimetric labelled substrate converted into product, a reporter gene that is responsive to changes in the functional activity of the hyperimmune serum reactive antigens and fragments thereof, and binding assays known in the art.

Another example of an assay for antagonists is a competitive assay that combines the hyperimmune serum reactive antigens and fragments thereof of the present invention and a potential antagonist with membrane-bound binding molecules, recombinant binding molecules, natural substrates or ligands, or substrate or ligand mimetics, under appropriate conditions for a competitive inhibition assay. The hyperimmune serum reactive antigens and fragments thereof can be labelled such as by radioactivity or a colorimetric compound, such that the molecule number of hyperimmune serum reactive antigens and fragments thereof bound to a binding molecule or converted to product can be determined accurately to assess the effectiveness of the potential antagonist.

Potential antagonists include small organic molecules, peptides, polypeptides and antibodies that bind to a hyperimmune serum reactive antigen and fragments thereof of the invention and thereby inhibit or extinguish its acitivity. Potential antagonists also may be small organic molecules, a peptide, a polypeptide such as a closely related protein or antibody that binds to the same sites on a binding molecule without inducing functional activity of the hyperimmune serum reactive antigens and fragments thereof of the invention.

Potential antagonists include a small molecule, which binds to and occupies the binding site of the hyperimmune serum reactive antigens and fragments thereof thereby preventing binding to cellular binding molecules, such that normal biological activity is prevented. Examples of small molecules include but are not limited to small organic molecules, peptides or peptide-like molecules.

Other potential antagonists include antisense molecules (see {Okano, H. et al., 1991}; OLIGODEOXYNUCLEOTIDES AS ANTISENSE INHIBITORS OF GENE EXPRESSION; CRC Press, Boca Ration, Fla. (1988), for a description of these molecules).

Preferred potential antagonists include derivatives of the hyperimmune serum reactive antigens and fragments thereof of the invention.

As used herein the activity of a hyperimmune serum reactive antigen and fragment thereof according to the present invention is its capability to bind to any of its interaction partner or the extent of such capability to bind to its or any interaction partner.

In a particular aspect, the invention provides the use of the hyperimmune serum reactive antigens and fragments thereof, nucleic acid molecules or inhibitors of the invention to interfere with the initial physical interaction between a pathogen and mammalian host responsible for sequelae of infection. In particular the molecules of the invention may be used: i) in the prevention of adhesion of H. pylori to mammalian extracellular matrix proteins on in-dwelling devices or to extracellular matrix proteins in mucosal wounds; ii) to block protein mediated mammalian cell invasion by, for example, initiating phosphorylation of mammalian tyrosine kinases {Rosenshine, I. et al., 1992} to block bacterial adhesion between mammalian extracellular matrix proteins and bacterial proteins which mediate tissue damage; iv) to block the normal progression of pathogenesis in infections initiated other than by the implantation of in-dwelling devices or by other surgical techniques.

Each of the DNA coding sequences provided herein may be used in the discovery and development of antibacterial compounds. The encoded protein upon expression can be used as a target for the screening of antibacterial drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest.

The antagonists and agonists may be employed, for instance, to inhibit diseases arising from infection with Helicobacter, especially H. pylori, such as peptic ulcer disease and gastric cancer.

In a still further aspect the present invention is related to an affinity device such affinity device comprises as least a support material and any of the hyperimmune serum reactive antigens and fragments thereof according to the present invention, which is attached to the support material. Because of the specificity of the hyperimmune serum reactive antigens and fragments thereof according to the present invention for their target cells or target molecules or their interaction partners, the hyperimmune serum reactive antigens and fragments thereof allow a selective removal of their interaction partner(s) from any kind of sample applied to the support material provided that the conditions for binding are met. The sample may be a biological or medical sample, including but not limited to, fermentation broth, cell debris, cell preparation, tissue preparation, organ preparation, blood, urine, lymph liquid, liquor and the like.

The hyperimmune serum reactive antigens and fragments thereof may be attached to the matrix in a covalent or non-covalent manner. Suitable support material is known to the one skilled in the art and can be selected from the group comprising cellulose, silicon, glass, aluminium, paramagnetic beads, starch and dextrane.

The present invention is further illustrated by the following figures, examples and the sequence listing from which further features, embodiments and advantages may be taken. It is to be understood that the present examples are given by way of illustration only and not by way of limitation of the disclosure.

In connection with the present invention

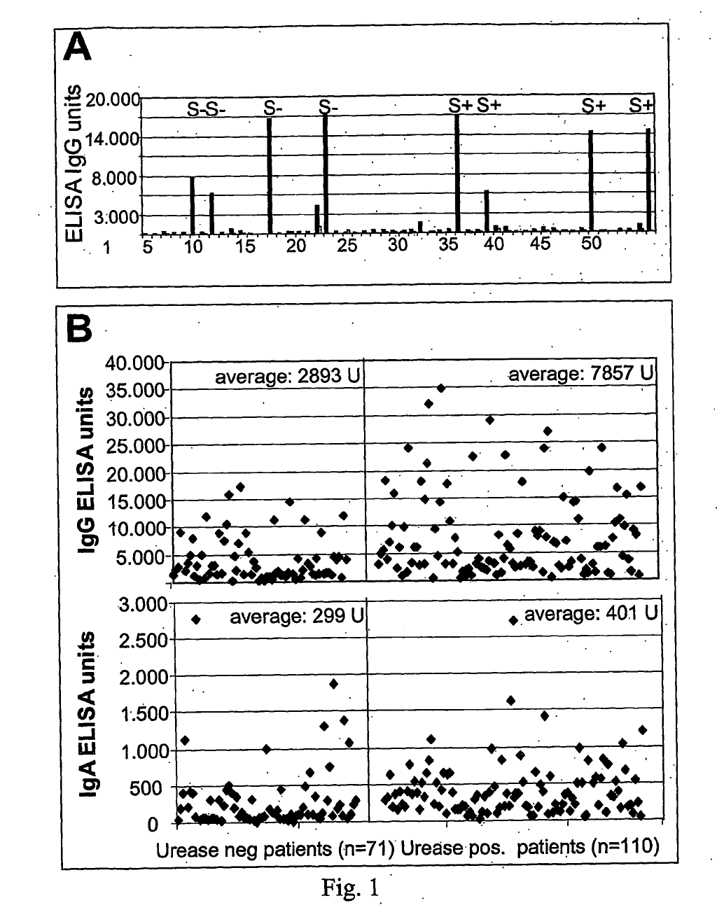

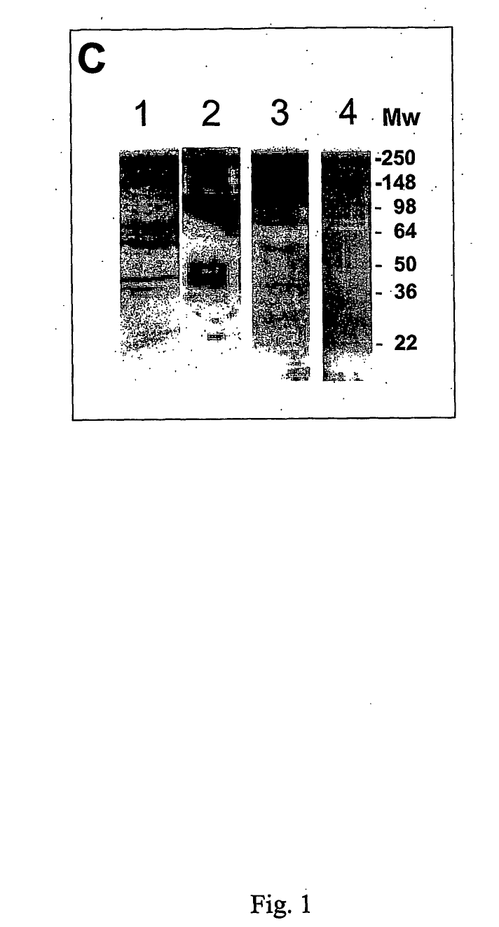

FIG. 1 shows the characterization of H. pylori specific human sera.

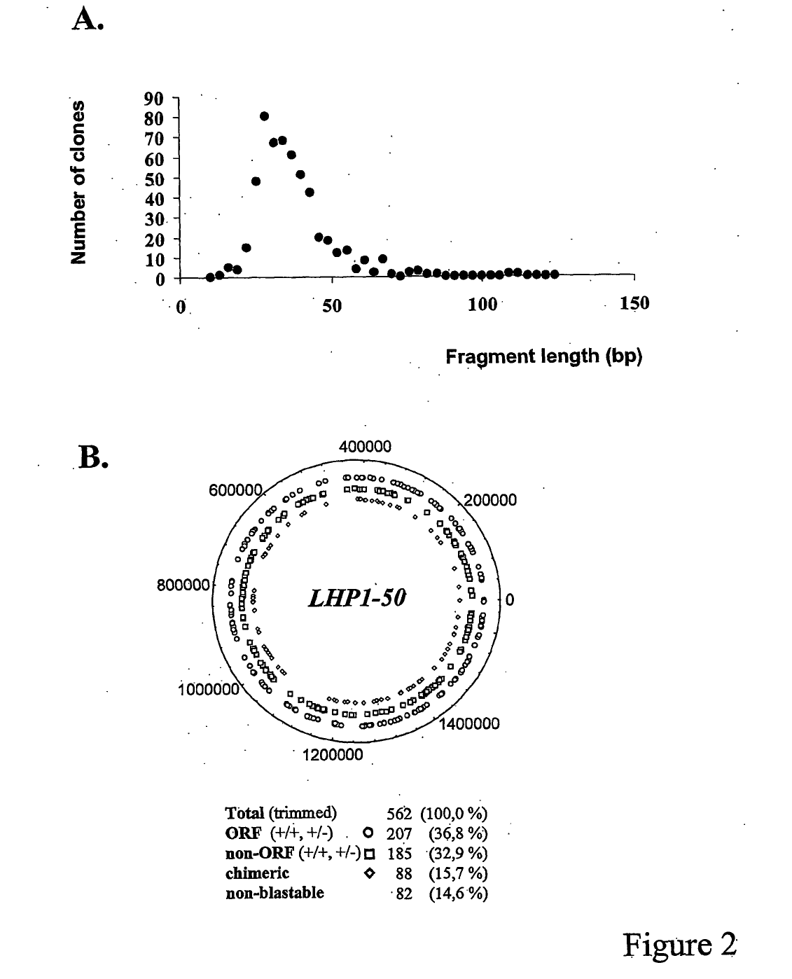

FIG. 2 shows the characterization of the small fragment genomic library LHP1-50 from H. pylori isolate KTH-Ca1.

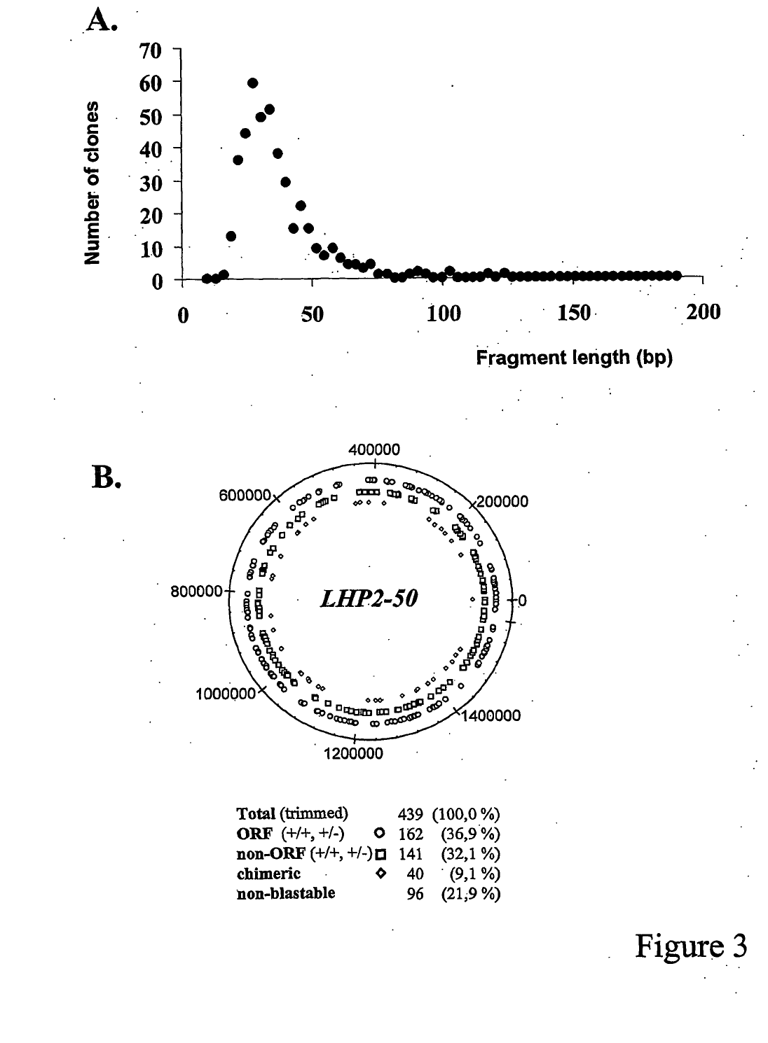

FIG. 3 shows the characterization of the small fragment genomic library LHP2-50 from H. pylori isolate KTH-Du.

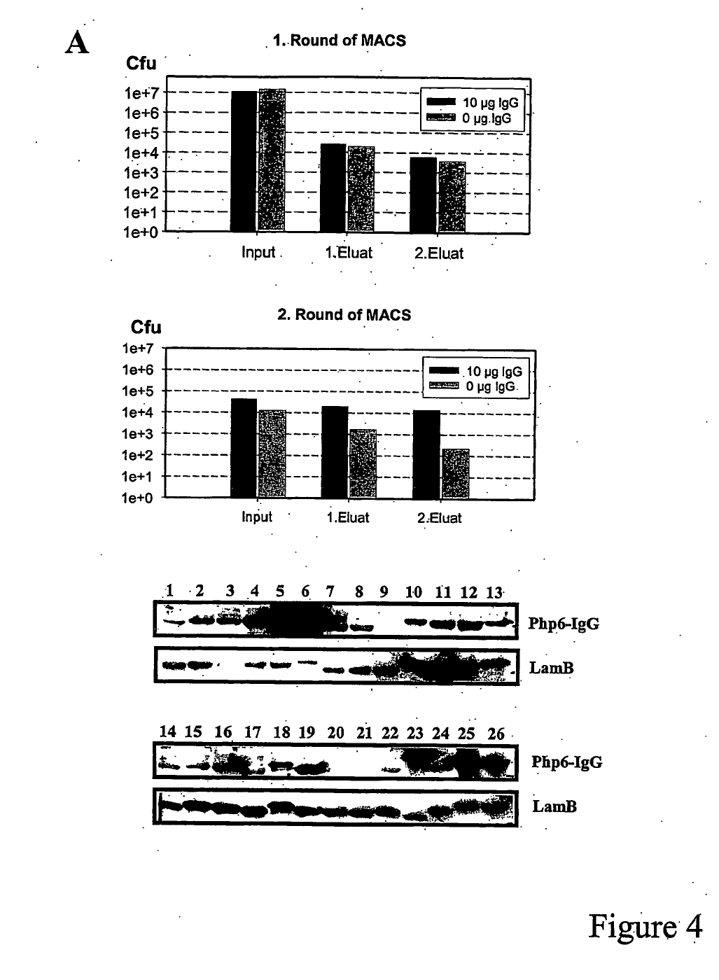

FIG. 4 shows the selection of bacterial cells by MACS using biotinylated human IgGs.

Table 1 shows the summary of all screens performed with genomic H. pylori libraries and human serum.

Table 2 shows the summary of all gene distribution experiments performed with genomic H. pylori DNA from individual isolates and gene specific oligonucleotides.

Table 3 shows the summary of epitope serology analysis with human sera.

The figures to which it might be referred to in the specification are described in the following in more details.

FIG. 1 shows the characterization of human sera for anti-H. pylori antibodies as measured by immune assays. Total anti-H. pylori IgG and IgA antibody levels were measured by standard ELISA using total bacterial lysate prepared from H. pylori KTH Ca1 strain as coating antigen. (A) Serum samples from randomly selected 54 adults were analysed for antibody levels and afterwards interviewed for symptoms (gasctric pain) and previous history of H. pylori infections. Four individuals were identified with high antibody titers without symptoms or disease (S−) and four with acute symptoms or known clinical disease (S+). (B) Sera from patients presenting themselves with typical symptoms of H. pylori diseases were analysed for IgG and IgA levels and grouped based on negative or positive results in the Urease test performed by clinicians. ELISA units are calculated from absorbance readings at two serum dilutions (10.000× and 50.000×). Averages for the two different groups are given. (C) Immunoblot analysis was performed on sera pre-selected by ELISA in order to ensure multiple immune reactivity with protein antigens. Results of a representative experiment using total bacterial lysate prepared from H. pylori KTH Ca1 strain and selected sera at 5.000× dilution are shown. Serum samples are from; Lane 1: a high titer S-individual, Lane 2: a high titer urease-patient, Lane 3 and 4: a high titer urease+patient. Lane 1-3 was developed with anti-human IgG secondary antibody and Lane 4 with anti-human IgA secondary antibody. Mw: molecular weigth markers.

FIG. 2 shows the fragment size distribution of the KTH-Ca1 H. pylori strain small fragment genomic library, LHP1-50. After sequencing 576 randomly selected clones sequences were trimmed to eliminate vector residues and the number of clones with various genomic fragment sizes were plotted. (B) Graphic illustration of the distribution of the same set of randomly sequenced clones of LHP2-50 over the H. pylori chromosome. Blue circles indicate matching sequences to annotated ORFs in both +/+ and +/− orientations. Red rectangles represent fully matched clones to non-coding chromosomal sequences in both +/+ and +/− orientations. Green diamonds positions all clones with chimeric sequences. Numeric distances in base pairs are indicated over each circular genome for orientation. Partitioning of various clone sets within the library is given in numbers and percentage at the bottom of the figure.

FIG. 3 shows the fragment size distribution of the KTH-Du H. pylori strain small fragment genomic library, LHP2-50. After sequencing 576 randomly selected clones sequences were trimmed to eliminate vector residues and the number of clones with various genomic fragment sizes were plotted. (B) Graphic illustration of the distribution of the same set of randomly sequenced clones of LHP2-50 over the H. pylori chromosome. Blue circles indicate matching sequences to annotated ORFs in both +/+ and +/− orientations. Red rectangles represent fully matched clones to non-coding chromosomal sequences in both +/+ and +/− orientations. Green diamonds positions all clones with chimeric sequences. Numeric distances in base pairs are indicated over each circular genome for orientation. Partitioning of various clone sets within the library is given in numbers and percentage at the bottom of the figure.

FIG. 4A shows the MACS selection with biotinylated human IgGs. The LHP1-50 library in pMAL9.1 was screened with 10 μg biotinylated, human serum (P6-IgG) in the first and with 10 μg in the second selection round. As negative control, no serum was added to the library cells for screening. Number of cells selected after the 1^{st} and 2^{nd} elution are shown for each selection round. FIG. 4B shows the reactivity of specific clones (1-26) isolated by bacterial surface display as analysed by Western blot analysis with the human serum (P6-IgG) used for selection by MACS at a dilution of 1:3,000. As a loading control the same blot was also analysed with antibodies directed against the platform protein LamB at a dilution of 1:5,000. LB, Extract from a done expressing LamB without foreign peptide insert.

Table 1: Immunogenic Proteins Identified by Bacterial Surface Display.

A, 300 bp library of H. pylori KTH Ca1 in fhuA with IC7-IgG (757), B, 300 bp library of H. pylori KTH Ca1 in fhuA with P5-IgG (729), C, 300 bp library of H. pylori KTH Ca1 in fhuA with P9-IgG (441), D, 50 bp library of H. pylori KTH Ca1 in lamB with IC7-IgG (448), E, 50 bp library of H. pylori KTH Ca1 in lamB with P5-IgA (1130), F, 50 bp library of H. pylori KTH Ca1 in lamB with P5-IgG (911), G, 50 bp library of H. pylori KTH Ca1 in lamB with P6-IgA (1135), H, 50 bp library of H. pylori KTH Ca1 in lamB with P6-IgG (844), I, 50 bp library of H. pylori KTH Ca1 in lamB with P9-IgG (1121), J, 300 bp library of H. pylori KTH Du in fhuA with P6-IgG (433), K, 300 bp library of H. pylori KTH Du in fhuA with P8-IgG (550), L, 50 bp library of H. pylori KTH Du in lamB with P6-IgG (1077), M, 50 bp library of H. pylori KTH Du in lamB with P8-IgG (740); *, prediction of antigenic sequences longer than 5 amino acids was performed with the program ANTIGENIC {Kolaskar, A. et al., 1990}.

Table 2: Gene Distribution in H. pylori strains.

28 H. pylori strains (including H. pylori KTH-Ca1 and KTH-Du, see example 2) and were tested by PCR with oligonucleotides specific for the genes encoding relevant antigens. The PCR fragment of one selected PCR fragment was sequenced in order to confirm the amplification of the correct DNA fragment. *, number of amino acid substitutions in a H. pylori strain derived from a cancer patient as compared to H. pylori strain KE26695 (aa, amino acids). #, an alternative strain was used for sequencing.

Table 3: Epitope Serology with Human Sera.

Immune reactivity of individual synthetic peptides representing selected epitopes with individual human sera is shown. Extent of reactivity is colour coded; white: neg (<500), light grey: + (500-650), dark grey: ++ (650-800), and black: +++ (>800). Numbers represent ELISA readings generated by measuring OD₄₀₅ₙₘ at serum dilution 200×. S stands for score, calculated as the sum of all reactivities (addition of the number of all +); P1 to P18 sera are from patients with definitive clinical diagnosis of duodenal or gastric ulcer, N1-4 sera are from healthy adults with high anti-H. pylori antibody titers without clinical symptomes of H. pylori disease. Location of synthetic peptides within the antigenic ORFs according to the genome annotation of H. pylori strain 26695 are given in columns from and to indicating the first and last amino acid residues, respectively. Peptide names: HP0009.1 present in annotated ORF HP0009.

### EXAMPLES

### Example 1

### Characterization and Selection of Human Sera Based Anti-H. pylori Antibodies, Preparation of Antibody Screening Reagents

Experimental Procedures

Enzyme Linked Immune Assay (ELISA).

ELISA plates (Maxisorb, Millipore) were coated with 5-10 μg/ml total protein diluted in coating buffer (0.1M sodium carbonate pH 9.2). Three dilutions of sera (2,000×, 10,000×, 50,000×) were made in PBS, BSA. Highly specific Horse Radish Peroxidase (HRP)-conjugated anti-human IgG or anti-human IgA secondary antibodies (Southern Biotech) were used according to the manufacturers' recommendations (dilution: 1,000×). Antigen-antibody complexes were quantified by measuring the conversion of the substrate (ABTS) to colored product based on OD₄₀₅ₙₘ readings by automatic ELIAS reader (TECAN SUNRISE).

Preparation of Bacterial Antigen Extracts

H. pylori KTH DU or KTH Ca1 strains were grown for 48 hours on agar plates, cells collected and lysed by repeated freeze-thaw cycles: incubation on dry ice/ethanol-mixture until frozen (1 min), then thawed at 37° C. (5 min): repeated 3 times. This was followed by sonication and collection of supernatant by centrifugation (3,500 rpm, 15 min, 4° C.).

Immunoblotting

Total bacterial lysate preparations were prepared from in vitro grown H. pylori KTH DU or KTH Ca1 strains. 10 to 25 μg total protein/lane was separated by SDS-PAGE using the BioRad Mini-Protean 3 Cell electrophoresis system and proteins transferred to nitrocellulose membrane (ECL, Amersham Pharmacia). After overnight blocking in 5% milk, human sera were added at 2,000× dilution, and HRPO labeled anti-human IgG was used for detection.

Purification of antibodies for genomic screening. Five sera from both the patient and the healthy group were selected based on the overall anti-streptococcal titers for a serum pool used in the screening procedure. Antibodies against E. coli proteins were removed by incubating the heat-inactivated sera with whole cell E. coli cells (DH5alpha, transformed with pHIE11, grown under the same condition as used for bacterial surface display). Highly enriched preparations of IgGs from the pooled, depleted sera were generated by protein G affinity chromatography, according to the manufacturer's instructions (UltraLink Immobilized Protein G, Pierce). IgA antibodies were purified also by affinity chromatography using biotin-labeled anti-human IgA (Southern Biotech) immobilized on Streptavidin-agarose (GIBCO BRL). The efficiency of depletion and purification was checked by SDS-PAGE, Western blotting, ELISA and protein concentration measurements.

Results

The antibodies produced against H. pylori by the human immune system and present in human sera are indicative of the in vivo expression of the antigenic proteins and their immunogenicity. These molecules are essential for the identification of individual antigens in the approach as described in the present invention, which is based on the interaction of the specific antibodies and the corresponding H. pylori peptides or proteins. To gain access to relevant antibody repertoires, human sera were collected from
I. 54 randomly selected healthy adults. Individuals were interviewed for the presence or absence of clinical symptoms and previously diagnosed H. pylori infection.
II. patients with duodenal ulcer.
III. patients with gastric ulcer and cancer.
   For the patient groups H. pylori infection was confirmed and medical diagnosis was based on medical microbiological tests, Urease test, biopsy or gastroscopy. A total of 191 sera from patients were included in the analysis.

The sera were characterized for anti-H. pylori antibodies by a series of ELISA and immunoblotting assays.

For that purpose two different antigen preparation were used: whole cell extracts prepared from H. pylori strains KTH-Ca1 and KTH-Du and both IgG and IgA antibody levels were determined. Antibody titers were expressed as units calculated from absorbance readings at two different dilutions—10.000× and 50.000× for IgG and 1.000× and 5.000× for IgA—where the response was linear (FIGS. 1A and B). Among the high titer randomly taken individuals eigth out of the 54 included (15%) showed significant IgG and IgA antibody levels. Half of these individuals were known H. pylori ‘patients’ acutely or before, the other half had no medical history or any complains. Sera of these four individuals were pooled and prepared for antigen identification. Since H. pylori infections are common, antibodies are present as a consequence of natural immunization from previous encounters with Helicobacter even without consequent carriage. The value of the ELISA assay imployed were further proved by analyzing patients' sera with or without active disease. Comparing the antibody levels in urease test positive and urease test negative individuals, significantly higher antibody levels were measured in the Urease+group (FIG. 1B). According to literature data, the false negative cases (˜10%) are much more prevalent then the false positives in this test, suggesting that the ELIAS assays is likely to be even more powerful predicting active H. pylori infections. Sera were ranked based on the reactivity against total lysate preparation in both antibody classes, and the highest ones from all three serum donor groups were selected for further analysis by immunoblotting. This latter assay confirmed immune reactivity against multiple H. pylori proteins, as it is examplified on FIG. 1C.

This extensive antibody characterization approach has led to the unambiguous identification of anti-Helicobacter hyperimmune sera and allowed the preparation of 5 donor pools.

### Example 2

### Generation of Highly Random, Frame-Selected, Small-Fragment, Genomic DNA Libraries of Helicobacter pylori

Experimental Procedures

Preparation of helicobacter genomic DNA. Sufficient amounts of bacterial pellets from the KTH-Ca1 and KTH-Du clinical isolates of H. pylori were obtained from Dr. Lars Engstrand. Bacterial pellets were washed 3× with PBS and carefully re-suspended in 0.5 ml of Lysozyme solution (100 mg/ml). 0.1 ml of 10 mg/ml heat treated RNase A and 20 U of RNase T1 were added, mixed carefully and the solution was incubated for 1 h at 37° C. Following the addition of 0.2 ml of 20% SDS solution and 0.1 ml of Proteinase K (10 mg/ml) the tube was incubated overnight at 55° C. 1/3 volume of saturated NaCl was then added and the solution was incubated for 20 min at 4° C. The extract was pelleted in a microfuge (13,000 rpm) and the supernatant transferred into a new tube. The solution was extracted with PhOH/CHCl₃/IAA (25:24:1) and with CHCl₃/IAA (24:1). DNA was precipitated at room temperature by adding 0.6× volume of Isopropanol, spooled from the solution with a sterile Pasteur pipette and transferred into tubes containing 80% ice-cold ethanol. DNA was recovered by centrifuging the precipitates with 10-12,000×g, then dried on air and dissolved in ddH₂O.

Preparation of small genomic DNA fragments. Genomic DNA was mechanically sheared into fragments ranging in size between 150 and 300 bp using a cup-horn sonicator (Bandelin Sonoplus UV 2200 sonicator equipped with a BB5 cup horn, 10 sec. pulses at 100% power output) or into fragments of size between 50 and 70 bp by mild DNase I treatment (Novagen). It was observed that sonication yielded a much tighter fragment size distribution when breaking the DNA into fragments of the 150-300 bp size range. However, despite extensive exposure of the DNA to ultrasonic wave-induced hydromechanical shearing force, subsequent decrease in fragment size could not be efficiently and reproducibly achieved. Therefore, fragments of 50 to 70 bp in size were obtained by mild DNase I treatment using Novagen's shotgun cleavage kit. A 1:20 dilution of DNase I provided with the kit was prepared and the digestion was performed in the presence of MnCl₂ in a 60 μl volume at 20° C. for 5 min to ensure double-stranded cleavage by the enzyme. Reactions were stopped with 2 μl of 0.5 M EDTA and the fragmentation efficiency was evaluated on a 2% TAE-agarose gel. This treatment resulted in total fragmentation of genomic DNA into near 50-70 bp fragments. Fragments were then blunt-ended twice using T4 DNA Polymerase in the presence of 100 μM each of dNTPs to ensure efficient flushing of the ends. Fragments were used immediately in ligation reactions or frozen at −20° C. for subsequent use.

Description of the vectors. The vector pMAL4.31 was constructed on a pASK-IBA backbone {Skerra, k, 1994} with the beta-lactamase (bla) gene exchanged with the Kanamycin resistance gene. In addition, the bla gene was cloned into the multiple cloning site. The sequence encoding mature beta-lactamase is preceded by the leader peptide sequence of ompA to allow efficient secretion across the cytoplasmic membrane. Furthermore a sequence encoding the first 12 amino acids (spacer sequence) of mature beta-lactamase follows the ompA leader peptide sequence to avoid fusion of sequences immediately after the leader peptidase cleavage site, since e.g. clusters of positive charged amino acids in this region would decrease or abolish translocation across the cytoplasmic membrane {Kajava, A. et al., 2000}. A SmaI restriction site serves for library insertion. An upstream FseI site and a downstream NotI site, which were used for recovery of the selected fragment, flank the SmaI site. The three restriction sites are inserted after the sequence encoding the 12 amino acid spacer sequence in such a way that the bla gene is transcribed in the −1 reading frame resulting in a stop codon 15 bp after the NotI site. A +1 bp insertion restores the bla ORF so that beta-lactamase protein is produced with a consequent gain of Ampicillin resistance.

The vector pMAL9.1 was constructed by cloning the lamB gene into the multiple cloning site of pEH1 {Hashemzadeh-Bonehi, L. et al., 1998}. Subsequently, a sequence was inserted in lamB after amino acid 154, containing the restriction sites FseI, SmaI and NotI. The reading frame for this insertion was constructed in such a way that transfer of frame-selected DNA fragments excised by digestion with FseI and NotI from plasmid pMAL4.31 yields a continuous reading frame of lamB and the respective insert.

The vector pHIE11 was constructed by cloning the fhuA gene into the multiple cloning site of pEH1. Thereafter, a sequence was inserted in fhuA after amino acid 405, containing the restriction site FseI, XbaI and NotI. The reading frame for this insertion was chosen in a way that transfer of frame-selected DNA fragments excised by digestion with FseI and NotI from plasmid pMAL4.31 yields a continuous reading frame of fhuA and the respective insert.

Cloning and evaluation of the library for frame selection. Genomic fragments of H. pylori DNA were ligated into the SmaI site of the vector pMAL4.31. Recombinant DNA was electroporated into DH10B electrocompetent E. coli cells (GIBCO BRL) and transformants plated on LB-agar supplemented with Kanamycin (50 μg/ml) and Ampicillin (50 μg/ml). Plates were incubated over night at 37° C. and colonies collected for large scale DNA extraction. A representative plate was stored and saved for collecting colonies for colony PCR analysis and large-scale sequencing. A simple colony PCR assay was used to initially determine the rough fragment size distribution as well as insertion efficiency. From sequencing data the precise fragment size was evaluated, junction intactness at the insertion site as well as the frame selection accuracy (3n+1 rule).

Cloning and evaluation of the library for bacterial surface display. Genomic DNA fragments were excised from the pMAL4.31 vector, containing the H. pylori libraries with the restriction enzymes FseI and NotI. The entire population of fragments was then transferred into plasmids pMAL9.1 (LamB) or pHIE11 (FhuA), which have been digested with FseI and NotI. Using these two restriction enzymes, which recognise an 8 bp GC rich sequence, the reading frame that was selected in the pMAL4.31 vector is maintained in each of the platform vectors. The plasmid library was then transformed into E. coli DH5alpha cells by electroporation. Cells were plated onto large LB-agar plates supplemented with 50 μg/ml Kanamycin and grown over night at 37° C. at a density yielding clearly visible single colonies. Cells were then scraped off the surface of these plates, washed with fresh LB medium and stored in aliquots for library screening at −80° C.

Results

Libraries for frame selection. Four libraries (LHP1-50, LHP2-50, LHP1-300 and LHP2-300) were generated in the pMAL4.31 vector with sizes of approximately 50 and 300 bp, respectively. For each library, ligation and subsequent transformation of approximately 1 μg of pMAL4.31 plasmid DNA and 50 ng of fragmented genomic H. pylori DNA yielded 4×10⁵ to 2×10⁶ clones after frame selection. To assess the randomness of the libraries, approximately 600 randomly chosen clones of LHP1-50 and LHP2-50 were sequenced. The bioinformatic analysis showed that of these clones only very few were present more than once. Furthermore, it was shown for example for LHP2-50 that 90% of the clones fell in the size range between 19 and 64 bp with an average size of 28 bp (FIG. 2, 3). All sequences followed the “3n+1 rule”, showing that all clones were properly frame selected.

Bacterial surface display libraries. The display of peptides on the surface of E. coli required the transfer of the inserts from the LHP libraries from the frame selection vector pMAL4.31 to the display plasmids pMAL9.1 (LamB) or pHIE11 (FhuA). Genomic DNA fragments were excised by FseI and NotI restriction and ligation of 5 ng inserts with 0.1 μg plasmid DNA and subsequent transformation into DH5alpha cells resulted in 2-5×10⁶ clones. The clones were scraped off the LB plates and frozen without further amplification.

### Example 3

### Identification of Highly Immunogenic Peptide Sequences from H. pylori Using Bacterial Surface Displayed Genomic Libraries and Human Serum

Experimental Procedures

MACS screening. Approximately 2.5×10⁸ cells from a given library were grown in 5 ml LB-medium supplemented with 50 μg/ml Kanamycin for 2 h at 37° C. Expression was induced by the addition of 1 mM IPTG for 30 min. Cells were washed twice with fresh LB medium and approximately 2×10⁷ cells re-suspended in 100 μl LB medium and transferred to an Eppendorf tube.

10 μg of biotinylated, human IgGs purified from serum was added to the cells and the suspension incubated over night at 4° C. with gentle shaking. 900 μl of LB medium was added, the suspension mixed and subsequently centrifuged for 10 min at 6,000 rpm at 4° C. (for IgA screens, 10 μg of purified IgAs were used and these captured with biotinylated anti-human-IgA secondary antibodies). Cells were washed once with 1 ml LB and then re-suspended in 100 μl LB medium. 10 μl of MACS microbeads coupled to streptavidin (Miltenyi Biotech, Germany) were added and the incubation continued for 20 min at 4° C. Thereafter 900 μl of LB medium was added and the MACS microbead cell suspension was loaded onto the equilibrated MS column (Miltenyi Biotech, Germany) which was fixed to the magnet. (The MS columns were equilibrated by washing once with 1 ml 70% EtOH and twice with 2 ml LB medium.)

The column was then washed three times with 3 ml LB medium. After removal of the magnet, cells were eluted by washing with 2 ml LB medium. After washing the column with 3 ml LB medium, the 2 ml eluate was loaded a second time on the same column and the washing and elution process repeated. The loading, washing and elution process was performed a third time, resulting in a final eluate of 2 ml.

A second round of screening was performed as follows. The cells from the final eluate were collected by centrifugation and re-suspended in 1 ml LB medium supplemented with 50 μg/ml Kanamycin. The culture was incubated at 37° C. for 90 min and then induced with 1 mM IPTG for 30 min. Cells were subsequently collected, washed once with 1 ml LB medium and suspended in 10 μl LB medium. Since the volume was reduced, 10 μg of human, biotinylated IgGs was added and the suspension incubated over night at 4° C. with gentle shaking. All further steps were exactly the same as in the first selection round. Cells selected after two rounds of selection were plated onto LB-agar plates supplemented with 50 μg/ml Kanamycin and grown over night at 37° C.

Evaluation of selected clones by sequencing and Western blot analysis. Selected clones were grown over night at 37° C. in 3 ml LB medium supplemented with 50 μg/ml Kanamycin to prepare plasmid DNA using standard procedures. Sequencing was performed at MWG (Germany).

For Western blot analysis approximately 10 to 20 μg of total cellular protein was separated by 10% SDS-PAGE and blotted onto HybondC membrane (Amersham Pharmacia Biotech, England). The LamB or FhuA fusion proteins were detected using human serum as the primary antibody at a dilution of approximately 1:5,000 and anti-human IgG or IgA antibodies coupled to HRP at a dilution of 1:5,000 as secondary antibodies. Detection was performed using the ECL detection kit (Amersham Pharmacia Biotech, England). Alternatively, rabbit anti FhuA or mouse anti LamB antibodies were used as primary antibodies in combination with the respective secondary antibodies coupled to HRP for the detection of the fusion proteins.

Results

Screening of bacterial surface display libraries by magnetic activated cell sorting (MACS) using biotinylated Igs. The libraries LHP1-50 and LHP2-50 in pMAL9.1 and LHP1-300 and LHP2-300 in pHIE11 were screened with pools of biotinylated, human IgGs and IgAs from patient sera or sera from healthy individuals (see Example 1: Preparation of antibodies from human serum). The selection procedure was performed as described under Experimental procedures. FIG. 4A shows a representative example of a screen with the LHP1-50 library and P6-IgGs. As can be seen from the colony count after the first selection cycle from MACS screening, the total number of cells recovered at the end is reduced from app. 1 to 2×10⁷ cells to approximately 10⁴ cells, whereby the selection in the presence of serum yielded a slightly higher number of cells than in the absence of antibodies (FIG. 4A). After the second round of screening however, a similar number of cells was recovered with P6-IgG, while only a few hundred cells were recovered when no IgGs from human serum were added, clearly showing that selection was dependent on H. pylori specific antibodies. To evaluate the performance of the screen, approximately 50 selected clones were picked randomly and subjected to Western blot analysis with the same, pooled serum (FIG. 4B). This analysis revealed that 70% of the selected clones showed reactivity with antibodies present in the relevant serum whereas the control strain expressing LamB without a H. pylori specific insert did not react with the same serum. In general, the rate of reactivity was observed to lie within the range of 35 to 75%. Colony PCR analysis confirmed that all selected clones contained an insert in the expected size range.

Subsequent sequencing of a larger number of randomly picked clones (600 to 1200 per screen) led to the identification of the gene and the corresponding peptide or protein sequence that was specifically recognized by the human serum used for screening. The frequency with which a specific clone is selected reflects at least in part the abundance and/or affinity of the specific antibodies in the serum used for selection and recognizing the epitope presented by this done. In that regard it is striking that clones derived from some ORFs (e.g. HP0527, HP0547 and HP1341) were picked more than 100 times, indicating their highly immunogenic property. Table 1 summarizes the data obtained for all 13 performed screens. All clones in Table 1 have been verified by Western blot analysis with whole cellular extracts from single clones to show the indicated reactivity with the pool of human serum used in the respective screen. As seen in Table 1, distinct regions of the identified ORF are identified as immunogenic, since variably sized fragments of the proteins are displayed on the surface by the platform proteins.

It is further worth noticing that most of the genes identified by the bacterial surface display screen encode proteins that are either attached to the surface of H. pylori and/or are secreted. This is in accordance with the expected role of surface attached or secreted proteins in virulence of H. pylori.

### Example 4

### Gene Distribution Studies with Highly Immunogenic Proteins Identified from H. pylori

Experimental Procedures

Gene distribution of H. pylori antigens by PCR. In order to establish whether the genes encoding the identified Helicobacter pylori antigens occur ubiquitously in H. pylori strains, PCR was performed on a series of independent H. pylori isolates with primers specific for the gene of interest. H. pylori isolates were obtained from patients covering various disease conditions associated with H. pylori infection (cancer patients: 11 strains, duodenal ulcer: 6, atrophic gastritis: 5, gastritis: 3, Hiatus hernia: 1, normal controls: 2). Oligonucleotide sequences as primers were designed for all identified ORFs yielding products of approximately 1,000 bp, if possible covering all identified immunogenic epitopes. Genomic DNA of all H. pylori strains was prepared as described under Example 2. PCR was performed in a reaction volume of 25 μl using Taq polymerase (1U), 200 nM dNTPs, 10 pMol of each oligonucleotide and the kit according to the manufacturers instructions (Invitrogen, The Netherlands). As standard, 30 cycles (1×: 5 min. 95° C., 30×: 30 sec. 95° C., 30 sec. 56° C., 30 sec. 72° C., 1×4 min. 72° C.) were performed, unless conditions had to be adapted for individual primer pairs.

Results

Identified genes encoding immunogenic proteins were tested by PCR for their presence in 28 different strains of H. pylori (Table 2). An ideal vaccine antigen would be an antigen that is present in all, or the vast majority of strains of the target organism to which the vaccine is directed. For a large number of antigens, the PCR reaction amplified a DNA fragment of the correct size with all 28 chosen H. pylori strains (e.g. HP0563, HP0887, HP1341). The sequencing of one PCR fragment from one individual strain showed that the amplified DNA fragment corresponds to the correct gene, but it also allows an estimation of the level of variation within this particular gene. While some genes possess a completely identical amino acid sequence in the two compared strains (e.g. HP0121, HP0413, HP1374), most antigens showed some degree of variation as listed in Table 2. The sequencing revealed that the amino acid sequences of approximately 80% of all antigens were to more than 95% identical in the two analysed strains, with only 1 strain showing a level of identity below 85%.

From a total of 106 genes analysed, 76 were present in all strains tested, 14 were present in more than 80% of the strains, while only 16 genes were absent in more than 20% of the tested 28 strains (Table 2). In addition, only 9 genes (e.g. HP115, HP717, HP887, HP913 and HP1119) showed a clear variation in size but were present in all or most H. pylori isolates. Importantly, many of the identified antigens are well conserved in all strains in sequence and size and are therefore novel vaccine candidates to prevent infections by H. pylori.

### Example 5

### Assessment of the Reactivity of Highly Immunogenic Peptide Sequences from H. pylori with Individual Human Sera

Experimental Procedures

Peptide Synthesis

Peptides were synthesized in small scale (4 mg resin; up to 288 in parallel) using standard F-moc chemistry on a Rink amide resin (PepChem, Tübingen, Germany) using a SyroII synthesizer (Multisyntech, Witten, Germany). After the sequence was assembled, peptides were elongated with Fmoc-epsilon-aminohexanoic acid (as a linker) and biotin (Sigma, St. Louis, Mo.; activated like a normal amino acid). Peptides were cleaved off the resin with 93% TFA, 5% triethylsilane, and 2% water for one hour. Peptides were dried under vacuum and freeze dried three times from acetonitrile/water (1:1). The presence of the correct mass was verified by mass spectrometry on a Reflex III MALDI-TOF (Bruker, Bremen Germany). The peptides were used without further purification.

Enzyme Linked Immune Assay (ELISA).

Biotin-labeled peptides (at the N-terminus) were coated on Streptavidin ELISA plates (EXICON) at 10 μg/ml concentration according to the manufacturer's instructions. Highly specific Horse Radish Peroxidase (HRP)-conjugated anti-human IgG secondary antibodies (Southern Biotech) were used according to the manufacturers' recommendations (dilution: 1,000×). Sera were tested at two serum dilutions, 200× and 1,000×. Following manual coating, peptide plates were processed and analyzed by the Gemini 160 ELISA robot (TECAN) with a built-in ELISA reader (GENIOS, TECAN).

Results

Following the bioinformatic analysis of selected clones, corresponding peptides were designed and synthesized. In case of epitopes with more than 26 amino acid residues, overlapping peptides were made. All peptides were synthesized with a N-terminal biotin-tag and used as coating reagents on Streptavidin-coated ELISA plates.

The analysis was performed with 144 peptides and 22 individual human serum samples which were included in the serum pools used for preparations of IgG and IgA screening reagents for bacterial surface display. A summary for serum reactivity of peptides representing H. pylori epitopes from the genomic screen analysed with human sera is shown in Table 3. The peptides were compared by the score calculated for each peptide based on the number of positive sera and the extent of reactivity. Peptides range from highly and widely reactive to weakly positive ones. Among the most reactive ones there are known antigens, some of them are also protective in animal challenge models, such as the CagA (HP0547) and vacuolating cytotoxin (HP0887). Besides the known antigens several novel highly immunogenic proteins and epitopes within those have been identified, such as the siderophore-mediated iron transport protein (HP1341), fumarate reductase flavoprotein subunit (frdA) (HP0192) and HP0087 hypothetical protein among others.

### REFERENCES

Altschul, S., et al. (1990). Journal of Molecular Biology 215: 403-10.
Bennett, D., et al. (1995). J Mol Recognit 8: 52-8.
Bjorkholm, B., et al. (2003). J Intern Med 253: 102-19.
Blaser, M. (2000). Nat Med 6: 376-7.
Censini, S., et al. (1996). Proc Natl Acad Sci USA 93:14648-53.
Clackson, T., et al. (1991). Nature 352: 624-8.
Devereux, J., et al. (1984). Nucleic acids research 12: 387-95.
Doherty, E., et al. (2001). Annu Rev Biophys Biomol Struct 30: 457-475.
Dubreuil, J., et al. (2002). Microbiol Mol Biol Rev 66: 617-29.
Eisenbraun, M., et al. (1993). DNA Cell Biol 12:791-7.
Etz, H., et al. (2001). J Bacteriol 183: 6924-35.
Ferrero, R., et al. (2001). Scand J Immunol 53:443-8.
Fujikawa, A., et al. (2003). Nat Genet 33: 375-81.
Ganz, T. (1999). Science 286:420-421.
Georgiou, G. (1997). Nature Biotechnology 15: 29-34.
Hancock, R. E., et al. (1999). Drugs 57: 469-473
Hashemzadeh-Bonehi, L., et al. (1998). Mol Microbiol 30: 676-678.
Heinje, von G (1987) e.g Sequence Analysis in Molecular Biology, Academic Press
Hemmer, B., et al. (1999). Nat Med 5: 1375-82.
Johanson, K., et al. (1995). J Biol Chem 270: 9459-71.
John Wiley & Sons (1987) Current Protocols in Molecular Biology
Jones, P., et al. (1986). Nature 321:522-5.
Kajava, A., et al. (2000). J Bacteriol 182: 2163-9.
Kersulyte, D., et al. (2000). J Bacteriol 182: 3210-8.
Kohler, G., et al. (1975). Nature 256: 495-7.
Kolaskar, A., et al. (1990). FEBS Lett 276:172-4.
Lewin, A., et al. (2001). Trends Mol Med 7:221-8.
Loughlin, M., et al. (2003). Infect Immun 71: 2022-31.
Marks, J., et al. (1992). Biotechnology (NY) 10: 779-83.
McCafferty, J., et al. (1990). Nature 348: 552-4.
Okano, H., et al. (1991). J Neurochem 56:560-7.
Oliaro, J., et al. (2000). J Med Microbiol 49: 643-50.
Oligodeoxynucleotides as Antisense Inhibitors of Gene Expression; CRC Press, Boca Ration, Fla. (1988) for a description of these molecules.
Prinz, C., et al. (2003). Trends Microbiol 11: 134-8.
Rammensee, H., et al. (1999). Immunogenetics 50: 213-9.
Rosenshine, I., et al. (1992). Infect Immun 60: 2211-7.
Sabarth, N., et al. (2002). Infect Immun 70: 6499-5303.
Seeger, C., et al. (1984). Proc Natl Acad Sci USA 81: 5849-52.
Shibuya, A., et al. (2000) Nature Immunology 1: 441-446
Skerra, A. (1994). Gene 151: 131-5.
Sutton, P. (2001). Vaccine 19: 2286-90.
Svennerholm, A. (2003). Vaccine 21:347-53.
Tang, D., et al. (1992). Nature 356: 152-4.
Tempest, P., et al. (1991). Biotechnology (NY) 9: 266-71.

Tourdot, S., et al. (2000). Eur J Immunol 30: 3411-21.

TABLE 1
Immunogenic proteins identified by bacterial surface display.
A, 300 bp library of H. pylori KTH Ca1 in fhuA with IC7-IgG (757), B, 300 bp library of H. pylori KTH Ca1 in fhuA
with P5-IgG (729), C, 300 bp library of H. pylori KTH Ca1 in fhuA with P9-IgG (441), D, 50 bp library of H. pylori
KTH Ca1 in lamB with IC7-IgG (448), E, 50 bp library of H. pylori KTH Ca1 in lamB with P5-IgA (1130), F, 50 bp
library of H. pylori KTH Ca1 in lamB with P5-IgG (911), G, 50 bp library of H. pylori KTH Ca1 in lamB with P6-
IgA (1135), H, 50 bp library of H. pylori KTH Ca1 in lamB with P6-IgG (844), I, 50 bp library of H. pylori KTH Ca1
in lamB with P9-IgG (1121), J, 300 bp library of H. pylori KTH Du in fhuA with P6-IgG (433), K, 300 bp library of
H. pylori KTH Du in fhuA with P8-IgG (550), L, 50 bp library of H. pylori KTH Du in lamB with P6-IgG (1077),
M, 50 bp library of H. pylori KTH Du in lamB with P8-IgG (740); *, prediction of antigenic sequences longer than 5
amino acids was performed with the program ANTIGENIC (Kolaskar and Tongaonkar, 1990).

| | | | No. of | Location | |
|---|---|---|---|---|---|
| | | | selected | of identified | |
| H. pylori | | | clones per | im- | Seq. |
| antigenic | Putative function | | ORF and | munogenic | ID (DNA, |
| protein | (by homology) | predicted immunogenic aa** | screen | region (aa) | Prot.) |
| HP0009 | outer membrane | 63-91, 95-101, 110-116, 134-148, 150-156, 158-164, | G: 1, H: 2, | 239-308 | 1, 179 |
| | protein (omp1) | 188-193, 197-209, 226-241, 247-254, 291-297, 312-319, | K: 2, M: 38 | | |
| | | 338-346, 351-358, 366-378, 404-410, 420-438, 448-454, | | | |
| | | 465-473, 482-488, 490-498, 503-510, 512-519, 531-543, | | | |
| | | 547-554, 568-575, 589-604, 610-631 | | | |
| HP0010 | chaperone and heat | 16-29, 35-47, 50-68, 70-79, 91-101, 143-149, 158-163, | K: 1 | 17-79, | 2, 180 |
| | shock protein | 185-191, 196-206, 215-224, 230-237, 244-251, 258-278, | | 218-233 | |
| | (groEL) | 290-311, 319-325, 338-351, 365-385, 396-429, 445-454, | | | |
| | | 458-466, 491-499, 501-521 | | | |
| HP0043 | mannose-6- | 4-10, 16-41, 46-66, 77-84, 91-97, 102-118, 125-144, 187-200, | G: 1 | 460-470 | 3, 181 |
| | phosphate | 202-214, 245-253, 255-261, 286-295, 300-330, 335-342, | | | |
| | isomerase (pmi) | 350-361, 363-381, 385-392, 396-416, 435-450 | | | |
| HP0063 | hypothetical protein | 11-19, 27-48, 52-59, 77-82, 84-107, 118-125, 127-154, | D: 4, E: 1, | 313-331 | 4, 182 |
| | | 178-183, 192-209, 215-221, 286-295, 302-313, 350-357, | G: 2 | | |
| | | 402-415, 417-431, 453-463, 465-493 | | | |
| HP0067 | urease accessory | 19-26, 30-43, 47-55, 63-68, 72-80, 97-104, 107-119, 129-146, | H: 1 | 26-43 | 5, 183 |
| | protein (ureH) | 160-175, 194-216, 231-251, 254-260 | | | |
| HP0072 | urease beta subunit | 7-13, 29-37, 65-81, 110-120, 123-131, 135-152, 230-249, | E: 2, G: 3, | 533-567 | 6, 184 |
| | (urea | 254-260, 284-290, 292-299, 317-326, 329-336, 403-444, | H: 1, M: 2 | | |
| | amidohydrolase) | 452-458, 466-477, 490-498, 510-519, 541-550, 557-566 | | | |
| | (ureB) | | | | |
| HP0086 | Conserved | 5-47, 71-77, 79-86, 89-95, 120-126, 137-144, 176-181, | B: 3, C: 1, I: 4 | 59-75 | 7, 185 |
| | hypothetical protein | 184-196, 202-208, 211-232, 236-282, 301-313, 317-325, | | | |
| | | 341-347, 353-384, 394-400, 412-433, 436-443 | | | |
| HP0087 | hypothetical protein | 4-18, 22-38, 59-69, 106-112, 116-130, 138-149, 156-170, | A: 2, B: 2, | 1-104, | 8, 186 |
| | | 175-197, 200-214, 216-223, 233-244, 255-261, 266-276, | E: 2, F: 1, | 130-147 | |
| | | 279-286, 325-333, 342-348, 366-399, 402-420, 429-441 | G: 2, I: 1, L: 2 | | |
| HP0088 | RNA polymerase | 50-58, 69-95, 97-113, 131-136, 157-163, 170-175, 188-212, | A: 2, B: 9, | 35-46 | 9, 187 |
| | sigma-70 factor | 220-226, 254-259, 265-277, 283-289, 297-308, 311-318, | D: 2, F: 3, | | |
| | (rpoD) | 347-358, 360-369, 378-401, 416-421, 440-450, 454-462, | H: 4 | | |
| | | 470-476, 493-502, 506-514, 536-567, 585-590, 598-607, | | | |
| | | 613-618, 653-659 | | | |
| HP0089 | pfs protein (pfs) | 16-29, 32-60, 65-87, 89-123, 128-134, 137-158, 162-173, | E: 11, G: 2 | 206-225 | 10, 188 |
| | | 178-196, 210-216, 218-228 | | | |
| HP0115 | flagellin B (flaB) | 10-20, 26-35, 51-64, 86-91, 94-100, 113-122, 154-160, | F: 2, H: 1, | 24-55, | 11, 189 |
| | | 185-191, 193-201, 211-217, 225-230, 237-246, 251-257, | K: 1, L: 52 | 236-341 | |
| | | 298-304, 306-312, 316-328, 340-348, 357-389, 391-397, | | | |
| | | 415-421, 449-456, 458-471, 488-495, 502-511 | | | |
| HP0175 | cell binding factor 2 | 5-22, 41-51, 87-93, 114-122, 127-136, 150-156, 158-166, | A: 11, C: 7, | 9-126, | 12, 190 |
| | | 223-233, 245-263, 291-296 | K: 2 | 127-285 | |
| HP0121 | phosphoenolpyruvate | 30-43, 46-56, 61-70, 72-83, 85-93, 103-113, 119-125, | H: 3, I: 4, K: 3 | 347-361 | 13, 191 |
| | synthase (ppsA) | 151-166, 179-191, 212-218, 225-231, 236-243, 262-267, | | | |
| | | 291-307, 331-344, 349-355, 366-372, 380-386, 414-422, | | | |
| | | 428-447, 459-464, 469-478, 507-519, 525-544, 563-569, | | | |
| | | 576-590, 620-626, 633-643, 654-659, 665-671, 684-707, | | | |
| | | 717-723, 725-733, 747-779, 782-801 | | | |
| HP0123 | threonyl-tRNA | 4-12, 14-26, 37-80, 107-115, 133-139, 144-150, 154-165, | D: 6, F: 1, | 7-23, | 14, 192 |
| | synthetase (thrS) | 173-180, 191-199, 205-211, 221-231, 237-244, 254-284, | H: 2, M: 1 | 465-479 | |
| | | 307-340, 342-353, 360-368, 370-380, 479-493, 495-503, | | | |
| | | 509-522, 525-536, 539-547, 554-560, 565-573, 578-583 | | | |
| HP0130 | hypothetical protein | 4-17, 47-55, 76-83, 85-100, 104-112, 117-123, 126-135, | A: 1, C: 1, I: | 258-283 | 15, 193 |
| | | 142-148, 156-167, 174-182, 267-273 | 1, K: 1, M: 3 | | |
| HP0150 | hypothetical protein | 8-32, 36-42, 65-88, 102-108, 112-140, 147-163, 170-179, | A: 1, B: 1, | 117-124 | 16, 194 |
| | | 183-193 | D: 1, J: 1, L: 2 | | |
| HP0183 | serine | 12-18, 45-50, 62-77, 82-95, 99-113, 115-123, 125-147, | I: 5 | 333-350 | 17, 195 |
| | hydroxymethyltransferase | 155-177, 187-209, 211-223, 244-253, 259-270, 278-297, | | | |
| | (glyA) | 302-307, 311-318, 329-334, 350-356, 359-365, 390-400, | | | |
| | | 402-413 | | | |
| HP0192 | fumarate reductase, | 4-13, 15-27, 30-46, 53-58, 68-74, 82-95, 115-126, 134-139, | H: 1, I: 4, | 376-400 | 18, 196 |
| | flavoprotein subunit | 148-153, 159-176, 182-199, 201-217, 220-225, 227-235, | L: 20 | | |
| | (frdA) | 237-248, 253-266, 300-315, 322-336, 390-396, 412-426, | | | |
| | | 438-445, 448-459, 477-484, 502-508, 515-527, 529-537, | | | |
| | | 553-568, 643-651, 658-667, 690-703 | | | |
| HP0197 | S- | 4-10, 24-32, 38-55, 59-67, 70-77, 80-87, 89-97, 123-129, | F: 1, H: 1, | 73-94 | 19, 197 |
| | adenosylmethionine | 134-151, 166-172, 178-189, 191-216, 218-235, 245-259, | L: 8 | | |
| | synthetase 2 (metX) | 271-315, 326-339, 341-360 | | | |
| HP0201 | fatty | 13-25, 31-38, 43-57, 79-85, 92-99, 106-112, 117-128, | L: 5 | 240-256 | 20, 198 |
| | acid/phospholipid | 130-139, 146-158, 160-175, 194-204, 211-222, 225-232, | | | |
| | synthesis protein | 234-242, 263-270, 278-292, 299-320, 322-333 | | | |
| | (plsX) | | | | |
| HP0202 | beta-ketoacyl-acyl | 4-17, 55-63, 66-101, 109-131, 135-143, 145-151, 155-161, | F: 1, I: 3 | 195-221 | 21, 199 |
| | carrier protein | 164-170, 177-185, 192-198, 213-218, 223-238, 246-256, | | | |
| | synthase III | 258-268, 273-283, 309-314, 322-328 | | | |
| HP0210 | chaperone and heat | 13-24, 31-39, 41-50, 63-69, 90-96, 104-109, 116-141, | F: 1, M: 2 | 344-363 | 22, 200 |
| | shock protein C62.5 | 148-153, 161-167, 173-178, 190-209, 253-258, 265-272, | | | |
| | (htpG) | 279-289, 295-312, 317-343, 355-366, 376-389, 400-407, | | | |
| | | 430-451, 453-464, 466-472, 487-493, 499-505, 523-538, | | | |
| | | 554-559, 568-579, 584-601 | | | |
| HP0211 | conserved | 5-22, 30-36, 53-59, 61-70, 82-92, 99-106, 120-131, 135-148, | A: 1, C: 1, | 111-249 | 23, 201 |
| | hypothetical | 154-167, 169-183, 187-199, 204-212, 231-247 | K: 1 | | |
| | secreted protein | | | | |
| HP0228 | conserved | 17-36, 40-66, 71-144, 148-171, 173-191, 199-214, 220-255, | A: 1, E: 6, | 287-307 | 24, 202 |
| | hypothetical | 265-272, 278-288, 298-333, 342-385 | F: 1 | | |
| | integral membrane | | | | |
| | protein | | | | |
| HP0229 | outer membrane | 4-16, 22-28, 30-36, 42-48, 95-116, 154-162, 164-174, | E: 1, F: 1, L: | 33-44, | 25, 203 |
| | protein (omp6) | 239-252, 258-263, 273-285, 306-313, 323-333, 341-357, | 14 | 233-258, | |
| | | 363-369, 372-379, 395-401, 430-436, 438-453, 464-480 | | 349-369 | |
| HP0235 | conserved | 4-21, 30-37, 46-53, 59-68, 80-92, 98-104, 118-143, 150-160, | B: 5 | 191-350 | 26, 204 |
| | hypothetical | 165-185, 187-200, 204-211, 224-236, 241-246, 252-258, | | | |
| | secreted protein | 271-280, 288-294, 311-320, 335-341 | | | |
| HP0239 | glutamyl-tRNA | 4-16, 37-59, 64-70, 79-87, 93-102, 107-127, 143-165, | H: 2 | 235-250 | 27, 205 |
| | reductase (hemA) | 172-188, 197-204, 207-218, 221-227, 242-248, 258-277, | | | |
| | | 289-296, 298-316, 332-338, 344-365, 367-373, 375-382, | | | |
| | | 400-408, 415-425, 438-446 | | | |
| HP0258 | conserved | 4-37, 39-66, 84-98, 101-127, 140-149, 157-163, 166-172, | H: 1, L: 1 | 183-204 | 28, 206 |
| | hypothetical | 175-182, 184-193, 203-208, 215-232, 234-247, 250-299, | | | |
| | integral membrane | 303-345 | | | |
| | protein | | | | |
| HP0266 | dihydroorotase | 10-20, 41-61, 73-87, 112-141, 176-192, 194-201, 205-222, | M: 21 | 304-328 | 29, 207 |
| | (pyrC) | 230-237, 257-264, 276-282, 284-310, 312-318 330-337, | | | |
| | | 349-357 | | | |
| HP0279 | lipopolysaccharide | 4-31, 42-103, 105-113, 121-153, 160-181, 188-196, 210-226, | G: 1, L: 6 | 304-318 | 30, 208 |
| | heptosyltransferase- | 231-264, 272-287, 297-304, 328-336 | | | |
| | 1 (rfaC) | | | | |
| HP0289 | toxin-like outer | 21-43, 46-52, 54-70, 72-79, 94-107, 133-141, 160-166, | B: 1, E: 2, | 511-523, | 31, 209 |
| | membrane protein | 217-253, 311-317, 359-365, 374-381, 390-395, 434-440, | H: 2, I: 3, L: 7 | 612-630, | |
| | | 488-494, 497-502, 511-522, 554-563, 565-574, 577-585, | | 1790-1803 | |
| | | 591-598, 601-606, 617-625, 633-643, 658-664, 676-682, | | | |
| | | 694-702, 710-719, 754-760, 782-788, 802-808, 916-921, | | | |
| | | 942-948, 955-964, 973-979, 992-998, 1006-1011, | | | |
| | | 1016-1023, 1030-1038, 1046-1053, 1059-1066, | | | |
| | | 1088-1098, 1119-1126, 1129-1135, 1156-1171, | | | |
| | | 1173-1181, 1202-1210, 1255-1261, 1268-1280, | | | |
| | | 1295-1310, 1312-1320, 1375-1381, 1406-1417, | | | |
| | | 1450-1471, 1478-1492, 1498-1506, 1569-1578, | | | |
| | | 1603-1608, 1611-1624, 1648-1655, 1663-1670, | | | |
| | | 1680-1698, 1702-1707, 1713-1719, 1737-1742, | | | |
| | | 1747-1753, 1762-1769, 1771-1785, 1790-1804, | | | |
| | | 1811-1818, 1830-1836, 1838-1852, 1874-1886, | | | |
| | | 1893-1899, 1902-1909, 1942-1948, 1952-1962, | | | |
| | | 1980-1986, 2001-2017, 2020-2028, 2042-2050, | | | |
| | | 2052-2068, 2074-2079, 2083-2095, 2107-2113, | | | |
| | | 2147-2155, 2177-2194, 2203-2211, 2236-2241, | | | |
| | | 2251-2258, 2267-2274, 2285-2292, 2314-2328, | | | |
| | | 2330-2340, 2358-2365, 2390-2401, 2408-2418, | | | |
| | | 2432-2453, 2463-2476, 2486-2507, 2528-2537, | | | |
| | | 2540-2548, 2552-2558, 2568-2576, 2596-2601, | | | |
| | | 2610-2622, 2629-2638, 2653-2669, 2718-2727, | | | |
| | | 2749-2767, 2777-2784, 2789-2795, 2806-2815, | | | |
| | | 2817-2824, 2835-2843, 2847-2854, 2860-2881 | | | |
| HP0292 | hypothetical protein | 4-54, 61-68, 72-82, 86-93, 100-108, 115-130, 147-154, | E: 14 | 96-109 | 32, 210 |
| | | 187-194, 196-207, 224-229, 236-251, 275-287 | | | |
| HP0295 | flagellin B homolog | 31-39, 62-69, 91-101, 158-172, 175-180, 186-193, 201-208, | A: 1, B: 2, | 366-503 | 33, 211 |
| | (fla) | 210-223, 243-250, 273-286, 293-299, 319-325, 343-354, | C: 3 | | |
| | | 356-365, 368-384, 414-435, 471-491, 512-518, 550-556, | | | |
| | | 567-581, 584-589, 633-639, 680-692, 697-708, 716-721, | | | |
| | | 747-754, 779-786, 810-816 | | | |
| HP0349 | CTP synthetase | 5-20, 22-48, 57-65, 96-101, 111-122, 130-145, 154-164, | M: 2 | 289-305 | 34, 212 |
| | (pyrG) | 170-181, 193-199, 201-216, 224-241, 244-262, 281-323, | | | |
| | | 342-351, 359-367, 369-396, 406-416, 424-433, 450-456, | | | |
| | | 485-491, 493-499, 501-515, 517-535 | | | |
| HP0351 | flagellar basal-body | 4-17, 22-44, 53-60, 66-83, 87-94, 101-106, 110-116, 131-137, | G: 9, I: 1 | 106-126 | 35, 213 |
| | M-ring protein (fliF) | 148-183, 189-207, 209-215, 233-242, 251-262, 264-272, | | | |
| | | 290-296, 308-327, 359-373, 375-380, 397-405, 415-420, | | | |
| | | 426-433, 444-475, 478-484, 529-536, 548-558 | | | |
| HP0380 | glutamate | 4-38, 42-50, 58-64, 72-81, 92-118, 140-146, 157-165, | H: 6, I: 1, L: 4 | 109-129, | 36, 214 |
| | dehydrogenase | 172-192, 198-204, 208-216, 227-234, 238-258, 271-278, | | 370-380 | |
| | (gdhA) | 288-293, 311-322, 327-346, 357-370, 375-383, 395-409, | | | |
| | | 411-417, 425-432, 436-445 | | | |
| HP0392 | histidine kinase | 23-30, 36-49, 52-64, 86-94, 97-104, 121-129, 257-272, | B: 1, F: 2 | 54-72 | 37, 215 |
| | (cheA) | 279-286, 288-294, 307-327, 334-340, 369-375, 377-386, | | | |
| | | 406-412, 418-423, 430-438, 441-447, 459-465, 469-476, | | | |
| | | 482-488, 510-546, 550-580, 584-622, 638-645, 653-659, | | | |
| | | 675-683, 692-705, 723-731, 752-761, 788-795 | | | |
| HP0401 | 3-phosphoshikimate | 11-33, 36-46, 88-104, 116-126, 134-170, 189-195, 199-217, | E: 1, M: 12 | 259-273 | 38, 216 |
| | 1- | 225-250, 255-261, 266-273, 280-291, 296-313, 334-341, | | | |
| | carboxyvinyltransferase | 343-349, 354-360, 362-369, 373-380, 387-401, 406-420 | | | |
| | (aroA) | | | | |
| HP0406 | hypothetical protein | 9-14, 28-44, 57-64, 72-79, 86-93, 104-111, 116-126, 142-150, | M: 2 | 61-86 | 39, 217 |
| | | 159-164 | | | |
| HP0409 | GMP synthase | 10-17, 26-33, 43-61, 69-95, 101-107, 109-125, 129-135, | B: 1, F: 1 | 60-74 | 40, 218 |
| | (guaA) | 137-144, 147-153, 158-169, 177-187, 209-219, 221-232, | | | |
| | | 235-247, 261-268, 271-282, 296-302, 306-347, 355-362, | | | |
| | | 364-379, 386-399, 409-418, 424-442, 451-460, 467-479, | | | |
| | | 490-498 | | | |
| HP0413 | transposase-like | 8-14, 20-31, 65-84, 94-99, 154-179, 193-207, 238-253 | E: 9, F: 1, G: 1 | 96-118 | 41, 219 |
| | protein, PS3IS | | | | |
| HP0459 | virB4 homolog | 4-24, 30-44, 47-62, 84-93, 108-116, 124-133, 136-141, | M: 6 | 167-189 | 42, 220 |
| | (virB4) | 201-209, 217-223, 228-235, 238-245, 247-270, 275-285, | | | |
| | | 290-314, 328-338, 342-349, 353-365, 375-383, 386-392, | | | |
| | | 394-402, 417-427, 443-459, 465-481, 492-514, 516-524, | | | |
| | | 550-566, 602-617, 630-639, 666-676, 687-693, 719-730, | | | |
| | | 747-753, 783-790, 799-816, 824-831, 837-842 | | | |
| HP0480 | GTP-binding | 6-15, 18-28, 58-66, 84-101, 106-129, 136-151, 154-165, | H: 1 | 362-375 | 43, 221 |
| | protein, fusA- | 182-203, 205-211, 214-220, 222-228, 233-240, 251-260, | | | |
| | homolog (yihK) | 270-277, 284-291, 306-315, 322-328, 363-369, 378-388, | | | |
| | | 392-405, 443-452, 495-501, 512-523, 574-583 | | | |
| HP0485 | catalase-like protein | 5-25, 27-34, 47-59, 64-70, 76-86, 145-158, 166-183, 189-202, | A: 1, B: 2, | 1-102 | 44, 222 |
| | | 217-231, 235-242, 260-270, 278-309 | C: 3, K: 2 | | |
| HP0508 | hypothetical protein | 4-19, 24-76, 78-83, 90-99, 102-109, 114-122, 137-147, | A: 1 | 226-325 | 45, 223 |
| | | 154-174, 177-188, 203-212, 217-223, 227-239 | | | |
| HP0519 | conserved | 7-37, 71-90, 94-109, 117-128, 141-153, 179-192, 199-206, | B: 2, H: 1 | 40-51 | 46, 224 |
| | hypothetical protein | 225-231, 237-243, 258-264 | | | |
| HP0525 | virB11 homolog | 13-19, 25-30, 46-59, 75-91, 101-107, 114-124, 129-135, | D: 5 | 282-300 | 47, 225 |
| | | 137-145, 160-167, 171-179, 187-194, 209-215, 217-222, | | | |
| | | 229-239, 243-249, 257-265, 269-275, 299-308, 310-327 | | | |
| HP0527 | cag pathogenicity | 86-100, 216-230, 342-369, 382-388, 424-430, 438-445, | A: 316, B: | 366-781, | 48, 226 |
| | island protein (cag7- | 452-458, 488-494, 501-518, 554-560, 568-574, 584-592, | 88, C: 61, F: | 782-1518, | |
| | cagY) | 603-609, 611-629, 639-645, 652-661, 669-699, 708-714, | 12, G: 1, H: | 1731-1747 | |
| | | 726-738, 747-753, 763-775, 785-791, 794-807, 815-824, | 1, L: 7, J: | | |
| | | 826-845, 854-860, 863-868, 870-883, 892-898, 901-906, | 274, K: 93, | | |
| | | 909-921, 930-937, 946-959, 968-974, 977-990, 998-1007, | L: 14, M: 12 | | |
| | | 1009-1027, 1037-1043, 1046-1051, 1053-1066, | | | |
| | | 1075-1081, 1084-1089, 1092-1103, 1113-1119, | | | |
| | | 1122-1135, 1143-1152, 1154-1172, 1182-1188, | | | |
| | | 1191-1196, 1200-1210, 1220-1226, 1229-1235, | | | |
| | | 1237-1249, 1259-1265, 1268-1281, 1289-1298, | | | |
| | | 1305-1318, 1328-1334, 1337-1343, 1345-1357, | | | |
| | | 1367-1373, 1390-1396, 1405-1411, 1418-1423, | | | |
| | | 1426-1435, 1445-1455, 1474-1483, 1493-1500, | | | |
| | | 1505-1512, 1517-1524, 1538-1544, 1568-1578, | | | |
| | | 1595-1601, 1674-1682, 1687-1720, 1728-1736, | | | |
| | | 1738-1744, 1754-1761, 1764-1774, 1798-1824, | | | |
| | | 1836-1842, 1886-1893, 1895-1903 | | | |
| HP0540 | cag pathogenicity | 4-17, 20-39, 46-55, 60-66, 102-110, 114-122, 125-131, | A: 2, B: 6, | 169-381 | 49, 227 |
| | island protein | 161-167, 172-178, 185-190, 195-202, 218-232, 236-252, | C: 2, I: 1 | | |
| | (cag19) | 264-291, 293-302, 309-315, 324-339 | | | |
| HP0541 | cag pathogenicity | 5-10, 13-40, 42-53, 69-75, 83-89, 120-135, 150-161, 174-190, | B: 2, G: 1 | 30-200 | 50, 228 |
| | island protein | 203-225, 229-247, 257-287, 318-348 | | | |
| | (cag20) | | | | |
| HP0542 | cag pathogenicity | 7-19, 43-53, 64-72, 124-139 | B: 2, L: 18, | 52-84, | 51, 229 |
| | island protein | | M: 2 | 120-131 | |
| | (cag21-cagG) | | | | |
| HP0544 | cag pathogenicity | 12-19, 39-48, 58-100, 117-123, 154-162, 164-187, 189-195, | H: 1 | 561-575 | 52, 230 |
| | island protein | 202-216, 218-235, 241-246, 262-278, 315-328, 333-347, | | | |
| | (cag23-cagE) | 354-366, 372-379, 391-405, 422-429, 431-442, 444-450, | | | |
| | | 458-466, 478-485, 494-501, 504-510, 520-535, 573-580, | | | |
| | | 589-598, 615-625, 666-676, 686-698, 722-729, 737-746, | | | |
| | | 756-767, 787-796, 805-816, 824-829, 833-848, 856-864, | | | |
| | | 866-876, 879-886, 898-904, 918-924, 927-934, 941-960, | | | |
| | | 967-978 | | | |
| HP0545 | cag pathogenicity | 11-29, 49-55, 70-77, 84-100, 102-112, 148-155, 160-177, | A: 1, B: 3, I: | 1-104 | 53, 231 |
| | island protein | 181-204 | 2, K: 1 | | |
| | (cag24) | | | | |
| HP547 | cag pathogenicity | 27-44, 64-71, 122-133, 151-156, 164-178, 214-220, 226-232, | A: 72, B: 65, | 110-357, | 54, 232 |
| | island protein | 235-244, 253-262, 282-288, 294-310, 317-325, 350-356, | C: 175, D: | 358-501, | |
| | (cag26-cagA) | 362-368, 376-383, 438-443, 449-454, 459-464, 492-498, | 74, F: 51, G: | 502-1161 | |
| | | 500-511, 529-535, 538-546, 567-573, 597-603, 660-665, | 7, H: 10, I: | | |
| | | 674-679, 724-734, 763-769, 773-784, 791-801, 807-815, | 108, J: 9, K: | | |
| | | 821-826, 840-848, 863-868, 897-902, 908-928, 932-953, | 23, L: 33, | | |
| | | 956-975, 980-987, 990-996, 1012-1018, 1042-1063, | M: 129 | | |
| | | 1095-1116, 1149-1157, 1160-1167 | | | |
| HP0563 | hypothetical protein | 4-21, 64-71, 73-84, 128-138, 144-162, 203-217, 240-263, | L: 134 | 330-345 | 55, 233 |
| | | 288-298, 300-308, 310-317, 325-351, 369-380, 391-411 | | | |
| HP0604 | uroporphyrinogen | 5-11, 25-31, 39-48, 51-79, 89-98, 100-122, 135-148, 166-201, | A: 2, B: 1, | 31-45 | 56, 234 |
| | decarboxylase | 203-227, 230-250, 254-260, 266-272, 274-282, 299-305, | F: 1, K: 2 | | |
| | (hemE) | 328-337 | | | |
| HP0607 | acriflavine | 12-23, 29-48, 51-60, 66-72, 75-81, 83-93, 103-115, 133-148, | F: 2, G: 1, I: | 915-940 | 57, 235 |
| | resistance protein | 168-174, 195-204, 222-229, 231-240, 242-251, 270-280, | 1, L: 2 | | |
| | (acrB) | 286-305, 322-344, 349-360, 364-370, 378-400, 421-441, | | | |
| | | 448-484, 486-493, 495-501, 504-534, 547-561, 567-590, | | | |
| | | 597-607, 621-635, 643-649, 658-685, 688-694, 702-711, | | | |
| | | 717-731, 737-742, 759-765, 767-772, 776-786, 803-809, | | | |
| | | 815-825, 854-908, 910-919, 923-930, 942-948, 961-975, | | | |
| | | 994-1014 | | | |
| HP0630 | modulator of drug | 4-9, 32-47, 51-61, 75-96, 139-191 | A: 1, F: 1, | 1-124 | 58, 236 |
| | activity (mda66) | | H: 2, I: 1 | | |
| HP0635 | hypothetical protein | 4-13, 17-38, 43-49, 55-76, 88-95, 110-121, 128-146, 151-157, | F: 2, H: 1 | 191-203 | 59, 237 |
| | | 162-214, 222-240, 243-249, 251-273, 275-281, 292-298, | | | |
| | | 300-309, 312-320, 322-331, 355-369, 376-408, 446-460, | | | |
| | | 471-482, 485-509 | | | |
| HP0655 | protective surface | 4-21, 72-82, 89-103, 106-115, 118-124, 140-146, 174-184, | G: 1, H: 2 | 33-51, | 60, 238 |
| | antigen D15 | 191-200, 204-213, 218-224, 261-266, 282-293, 299-309, | | 818-835 | |
| | | 311-340, 342-358, 362-372, 381-389, 391-402, 413-421, | | | |
| | | 438-447, 457-464, 470-478, 501-507, 545-560, 578-624, | | | |
| | | 631-641, 658-670, 680-689, 717-738, 753-759, 795-805, | | | |
| | | 816-822, 830-838, 842-848, 869-881, 892-898 | | | |
| HP0659 | hypothetical protein | 4-21, 79-85, 156-177, 183-188, 206-214, 243-249, 261-269, | A: 4, B: 1, | 37-97, | 61, 239 |
| | | 287-292, 315-322, 334-345, 360-366, 374-390, 402-411 | K: 2 | 260-399 | |
| HP0683 | UDP-N- | 4-9, 19-54, 58-78, 97-104, 111-120, 126-134, 137-145, | G: 8 | 240-249 | 62, 240 |
| | acetylglucosamine | 163-173, 178-188, 193-203, 211-224, 246-286, 288-324, | | | |
| | pyrophosphorylase | 337-346, 355-362, 374-390, 392-398, 409-417 | | | |
| | (glmU) | | | | |
| HP0687 | iron(II) transport | 5-12, 14-31, 35-41, 43-61, 82-92, 97-105, 134-145, 155-166, | F: 1, L: 3 | 144-154 | 63, 241 |
| | protein (feoB) | 184-203, 215-223, 225-251, 272-279, 281-306, 310-345, | | | |
| | | 358-418, 435-473, 482-490, 525-532, 538-547, 549-563, | | | |
| | | 578-604, 613-639 | | | |
| HP0696 | N- | 53-59, 64-72, 74-100, 133-152, 154-172, 176-181, 207-214, | A: 5, B: 6, | 544-685 | 64, 242 |
| | methylhydantoinase | 225-238, 275-297, 304-310, 331-340, 362-367, 384-395, | C: 13, E: 2, | | |
| | | 403-410, 437-443, 448-456, 482-490, 579-597, 602-610, | F: 1, G: 1, L: 1 | | |
| | | 625-630, 633-651, 699-707, 709-715, 734-743, 750-762 | | | |
| HP0701 | DNA gyrase, sub A | 12-18, 22-40, 45-83, 89-97, 103-109, 147-153, 159-173, | G: 2, H: 2, I: | 202-218, | 65, 243 |
| | (gyrA) | 195-204, 210-219, 243-253, 259-265, 273-282, 303-309, | 3, L: 1 | 282-299, | |
| | | 315-325, 332-340, 346-358, 362-367, 377-390, 393-402, | | 339-350, | |
| | | 418-426, 447-455, 467-480, 505-512, 514-525, 548-561, | | 617-628 | |
| | | 566-576, 584-596, 619-626, 638-645, 649-659, 661-680, | | | |
| | | 699-708, 714-720, 753-759, 766-772, 775-781, 801-808 | | | |
| HP0706 | outer membrane | 5-33, 52-62, 87-101, 111-135, 137-143, 145-152, 190-202, | J: 1, K: 1, L: | 151-215 | 66, 244 |
| | protein (omp15) | 209-221, 233-245, 253-270 | 37 | | |
| HP0714 | RNA polymerase | 19-29, 32-39, 42-48, 75-94, 124-135, 137-145, 152-160, | F: 7 | 48-64 | 67, 245 |
| | sigma-54 factor | 176-182, 193-203, 215-236, 266-273, 275-291, 297-306, | | | |
| | (rpoN) | 311-319, 322-342, 348-360, 369-378, 394-401 | | | |
| HP0717 | DNA polymerase III | 4-11, 13-33, 36-43, 53-63, 65-80, 112-129, 134-141, 143-155, | A: 2, B: 3, | 390-516 | 68, 246 |
| | gamma and tau | 157-168, 178-188, 191-199, 201-207, 215-229, 242-255, | M: 2 | | |
| | subunits (dnaX) | 263-270, 283-315, 320-329, 333-338, 340-349, 412-426, | | | |
| | | 465-478, 485-490, 498-512, 540-554 | | | |
| HP0723 | L-asparaginase II | 4-18, 23-32, 41-47, 54-70, 88-99, 104-111, 118-138, 143-148, | B: 1, F: 1, | 1-90 | 69, 247 |
| | (ansB) | 150-162, 168-175, 181-188, 203-211, 214-220, 227-245, | H: 6, K: 2 | | |
| | | 251-268, 275-281, 287-296, 323-333 | | | |
| HP0727 | transcriptional | 8-34, 38-49, 72-83, 85-91, 94-104, 112-125, 134-142, | B: 2, G: 2, I: 2 | 141-159 | 70, 248 |
| | regulator, putative | 148-168, 181-189, 191-198, 202-214, 222-233, 242-254, | | | |
| | | 256-262, 273-278, 287-294, 314-325 | | | |
| HP0752 | flagellar hook- | 4-24, 30-36, 47-75, 82-105, 124-134, 151-157, 192-202, | A: 7, B: 4, | 64-84, | 71, 249 |
| | associated protein 2 | 208-214, 219-226, 234-247, 285-290, 318-324, 332-340, | C: 2, G: 1, | 93-180, | |
| | (fliD) | 343-349, 380-386, 453-462, 472-478, 484-501, 531-540, | H: 1, I: 3, J: 1, | 181-446 | |
| | | 550-557, 604-612, 620-625, 642-648, 652-671 | K: 2 | | |
| HP0760 | conserved | 12-18, 24-32, 68-75, 77-83, 96-101, 109-116, 129-136, | F: 3, G: 1 | 364-374 | 72, 250 |
| | hypothetical protein | 152-164, 175-184, 190-199, 206-215, 224-233, 241-250, | | | |
| | | 258-264, 273-292, 302-312, 319-331, 334-346, 348-368, | | | |
| | | 387-395, 408-416, 420-429, 437-452 | | | |
| HP0836 | hypothetical protein | 11-28, 36-52, 60-67, 74-79, 108-116 | B: 1, L: 4 | 61-76 | 73, 251 |
| HP0850 | type I restriction | 20-27, 38-49, 69-74, 84-107, 138-145, 161-168, 179-195, | H: 2 | 146-160 | 74, 252 |
| | enzyme M protein | 210-226, 228-252, 267-281, 283-296, 305-311, 333-340, | | | |
| | (hsdM) | 342-356, 361-372, 380-399, 401-414, 458-466, 475-481, | | | |
| | | 492-507, 515-520 | | | |
| HP0853 | ABC transporter, | 43-61, 68-74, 76-90, 120-128, 130-149, 156-161, 164-182, | M: 15 | 261-285 | 75, 253 |
| | ATP-binding | 206-234, 242-252, 269-274, 291-304, 332-345, 349-355, | | | |
| | protein (yheS) | 360-371, 374-388, 434-440, 447-453, 459-465, 469-496, | | | |
| | | 504-522 | | | |
| HP0863 | hypothetical protein | 4-17, 24-30, 37-49, 87-98, 118-124, 126-136, 144-171, | E: 1, M: 11 | 401-427 | 76, 254 |
| | | 176-188, 206-214, 216-228, 233-240, 246-252, 262-271, | | | |
| | | 277-297, 307-330, 333-342, 346-352, 355-361, 368-386, | | | |
| | | 391-400, 413-420, 474-480 | | | |
| HP0874 | hypothetical protein | 15-26, 31-46, 51-72, 80-93, 96-109, 131-137, 150-158, | F: 2 | 173-190 | 77, 255 |
| | | 179-185, 189-209, 211-219, 221-234, 241-247, 255-262, | | | |
| | | 265-271, 283-288 | | | |
| HP0875 | catalase | 28-37, 39-45, 51-58, 77-84, 89-97, 132-148, 171-180, | K: 6 | 425-497 | 78, 256 |
| | | 199-205, 212-218, 220-226, 257-265, 273-300, 307-327, | | | |
| | | 334-340, 344-365, 385-390, 402-408, 426-436, 450-468, | | | |
| | | 476-485 | | | |
| HP0876 | iron-regulated outer | 4-25, 70-76, 80-88, 90-100, 120-128, 162-169, 183-203, | B: 1, E: 1, | 497-509 | 79, 257 |
| | membrane protein | 261-277, 279-289, 291-297, 302-308, 321-327, 339-353, | G: 1, I: 1 | | |
| | (frpB) | 358-377, 392-401, 404-410, 414-422, 443-450, 456-461, | | | |
| | | 470-488, 490-497, 510-535, 570-611, 618-630, 639-647, | | | |
| | | 649-660, 668-690, 702-716, 718-724, 737-747, 750-764 | | | |
| HP0887 | vacuolating | 12-48, 50-64, 99-108, 216-223, 235-241, 244-254, 262-274, | A: 1, B: 5, | 76-100, | 80, 258 |
| | cytotoxin | 287-293, 310-316, 320-326, 361-366, 377-383, 390-395, | C: 2, D: 3, | 270-284, | |
| | | 408-414, 418-425, 438-444, 462-469, 494-505, 524-530, | E: 5, G: 2, | 309-438, | |
| | | 536-547, 551-566, 592-598, 601-613, 678-685, 687-695, | H: 3, I: 2, K: | 493-505, | |
| | | 709-717, 727-737, 751-757, 760-765, 772-778, 782-788, | 9, L: 29, M: 7 | 786-942, | |
| | | 801-807, 822-830, 859-868, 870-878, 884-890, 898-903, | | 947-967 | |
| | | 909-919, 953-969, 973-980, 990-1000, 1002-1019, | | | |
| | | 1041-1047, 1059-1065, 1090-1095, 1116-1127, | | | |
| | | 1130-1139, 1143-1149, 1151-1168, 1178-1183, | | | |
| | | 1188-1195, 1197-1209, 1213-1220, 1226-1234, | | | |
| | | 1236-1247, 1255-1274, 1276-1282 | | | |
| HP0891 | conserved | 4-9, 24-34, 46-95, 97-109, 119-130 | F: 2 | 138-156 | 81, 259 |
| | hypothetical protein | | | | |
| HP0910 | adenine specific | 9-26, 28-35, 43-53, 55-68, 83-92, 99-105, 110-135, 139-149, | E: 33, H: 1 | 92-107, | 82, 260 |
| | DNA | 157-162, 164-170, 173-183, 193-208, 210-230, 239-245, | | 154-173 | |
| | methyltransferase | 253-259, 263-271, 293-305, 310-320, 322-331, 336-343, | | | |
| | (HINDIIM) | 351-364, 367-376 | | | |
| HP0913 | outer membrane | 19-39, 52-62, 108-117, 145-152, 160-168, 194-203, 229-240, | C: 1, I: 1 | 247-260 | 83, 261 |
| | protein (omp21) | 252-268, 280-287, 308-316, 333-339, 383-390, 403-412, | | | |
| | | 414-424, 438-445, 464-472, 479-484, 489-505, 510-526 | | | |
| HP0922 | toxin-like outer | 5-17, 25-52, 60-77, 105-113, 118-125, 162-167, 228-234, | A: 1, B: 3, | 394-549 | 84, 262 |
| | membrane protein | 272-279, 328-334, 341-357, 381-395, 400-406, 512-518, | D: 1, F: 4, | | |
| | | 557-569, 586-592, 645-651, 690-695, 701-709, 720-726, | G: 2 | | |
| | | 733-743, 751-758, 781-786, 879-886, 929-934, 939-944, | | | |
| | | 952-960, 965-975, 994-1001, 1039-1045, 1102-1109, | | | |
| | | 1164-1181, 1198-1206, 1223-1229, 1253-1259, | | | |
| | | 1283-1292, 1312-1317, 1339-1349, 1360-1370, | | | |
| | | 1389-1398, 1400-1412, 1452-1465, 1470-1484, | | | |
| | | 1490-1497, 1519-1525, 1554-1564, 1578-1591, | | | |
| | | 1623-1636, 1638-1646, 1669-1679, 1685-1697, | | | |
| | | 1704-1711, 1713-1720, 1730-1736, 1738-1749, | | | |
| | | 1756-1764, 1778-1786, 1796-1803, 1817-1826, | | | |
| | | 1849-1866, 1975-1993, 2017-2032, 2044-2053, | | | |
| | | 2070-2086, 2091-2109, 2116-2127, 2156-2167, | | | |
| | | 2182-2188, 2197-2202, 2244-2252, 2281-2287, | | | |
| | | 2290-2307, 2350-2361, 2383-2404, 2425-2433, | | | |
| | | 2445-2455, 2495-2505 | | | |
| HP0925 | recombinational | 9-24, 31-53, 57-67, 69-79, 84-114, 133-141, 144-172, | E: 1, G: 1 | 13-46 | 85, 263 |
| | DNA repair protein | 178-186 | | | |
| | (recR) | | | | |
| HP0953 | hypothetical protein | 4-25, 27-35, 43-52, 59-70, 79-91, 115-130, 136-152, 154-163, | J: 3 | 1-58 | 86, 264 |
| | | 170-179 | | | |
| HP0973 | hypothetical protein | 4-30, 49-55, 71-80, 96-105, 111-126, 139-146, 149-162, | B: 3, K: 2 | 250-351 | 87, 265 |
| | | 239-245, 279-285, 290-296, 300-307, 331-337, 343-350 | | | |
| HP0977 | conserved | 9-27, 34-41, 43-51, 92-111, 114-120, 123-131, 139-150, | A: 2 | 214-398 | 88, 266 |
| | hypothetical | 156-171, 176-186, 188-204, 229-241, 252-258, 266-279, | | | |
| | secreted protein | 288-297, 319-334, 338-348, 373-379, 389-398, 431-439, | | | |
| | | 479-484 | | | |
| HP1019 | serine protease | 4-15, 18-27, 47-52, 68-83, 91-97, 104-110, 115-121, 139-147, | A: 3, H: 1, | 232-386 | 89, 267 |
| | (htrA) | 157-164, 198-206, 227-236, 241-254, 264-273, 278-289, | I: 1 | | |
| | | 311-320, 353-361, 372-383, 405-420, 426-434 | | | |
| HP1024 | co-chaperone- | 4-10, 24-34, 91-97, 129-141, 156-163, 184-190, 205-219, | E: 1, G: 1, | 93-116 | 90, 268 |
| | curved DNA | 229-235, 256-273, 278-285 | L: 24 | | |
| | binding protein A | | | | |
| | (CbpA) | | | | |
| HP1052 | UDP-3-0-acyl N- | 7-29, 35-54, 71-83, 85-91, 104-111, 122-134, 138-144, | E: 1, F: 5, G: | 48-66 | 91, 269 |
| | acetylglcosamine | 146-154, 158-174, 177-183, 186-201, 207-215, 223-235, | 14 | | |
| | deacetylase (envA) | 240-247, 262-273, 275-283, 287-292 | | | |
| HP1090 | cell division protein | 7-27, 31-47, 49-70, 75-102, 110-149, 157-171, 217-223, | D: 2, F: 1, | 475-490 | 92, 270 |
| | (ftsK) | 235-251, 294-302, 358-364, 367-375, 387-393, 395-412, | G: 1, H: 1 | | |
| | | 423-430, 441-451, 456-470, 472-486, 488-495, 499-509, | | | |
| | | 515-529, 536-549, 556-570, 574-603, 607-615, 625-633, | | | |
| | | 642-658, 670-676, 683-702, 708-716, 720-726, 747-756, | | | |
| | | 763-784, 803-812, 815-826 | | | |
| HP1098 | conserved | 7-22, 30-38, 53-59, 64-75, 83-95, 97-112, 120-131, 133-142, | A: 4, B: 1, | 9-156, | 93, 271 |
| | hypothetical | 145-151, 154-166, 172-180, 189-203, 227-238, 277-287 | F: 1, I: 2, K: 1 | 174-287 | |
| | secreted protein | | | | |
| HP1116 | hypothetical protein | 13-23, 25-32, 111-117, 150-164, 185-193, 207-212, 216-224, | I: 1, K: 2, L: | 66-86 | 94, 272 |
| | | 230-236, 263-272, 304-311, 342-348, 374-385, 391-407, | 31 | | |
| | | 444-458, 480-487, 489-499, 523-542, 544-558, 572-579, | | | |
| | | 620-640, 686-696, 703-710, 742-755, 765-772, 817-822, | | | |
| | | 830-837, 865-872, 931-937 | | | |
| HP1117 | conserved | 4-27, 49-56, 62-70, 86-92, 121-127, 151-163, 170-182, | A: 1, F: 4 | 234-254 | 95, 273 |
| | hypothetical | 195-202, 212-226, 237-243 | | | |
| | secreted protein | | | | |
| HP1119 | flagellar hook- | 4-10, 13-24, 39-51, 62-78, 92-104, 107-117, 134-141, | A: 7, B: 1, | 252-483 | 96, 274 |
| | associated protein 1 | 156-161, 166-181, 210-216, 222-229, 256-266, 273-280, | D: 1, F: 3, J: 1 | | |
| | (HAP1) (flgK) | 297-304, 313-330, 336-349, 371-376, 433-439, 443-448, | | | |
| | | 488-493, 506-515, 527-534, 560-572, 575-583, 587-593 | | | |
| HP1126 | colicin tolerance-like | 4-15, 21-38, 45-56, 81-95, 102-108, 118-130, 133-147, | A: 3, B: 2, J: 1 | 24-146 | 97, 275 |
| | protein (tolB) | 152-162, 166-171, 199-204, 211-218, 230-240, 253-261, | | | |
| | | 274-283, 288-294, 312-317, 325-336, 344-357, 391-414 | | | |
| HP1152 | signal recognition | 26-31, 38-56, 65-82, 90-101, 112-119, 123-153, 175-188, | D: 1, F: 4, | 290-306 | 98, 276 |
| | particle protein (ffh) | 197-216, 234-242, 249-265, 273-286, 290-305, 327-335, | G: 10 | | |
| | | 338-346, 361-372, 394-404 | | | |
| HP1153 | valyl-tRNA | 17-26, 43-48, 50-73, 81-93, 95-107, 139-146, 158-168, | F: 4, G: 3, L: 3 | 820-851 | 99, 277 |
| | synthetase (valS) | 171-176, 190-196, 202-212, 216-223, 243-266, 274-282, | | | |
| | | 308-313, 324-330, 344-378, 380-387, 403-422, 427-443, | | | |
| | | 448-455, 457-465, 491-515, 517-528, 553-567, 589-599, | | | |
| | | 610-617, 642-648, 670-697, 709-717, 726-743, 745-759, | | | |
| | | 769-803, 807-823, 840-849 | | | |
| HP1186 | carbonic anhydrase | 4-18, 39-48, 53-63, 66-90, 102-117, 125-134, 137-145, | D: 5, H: 1 | 26-40, | 100, 278 |
| | | 156-162, 169-197 | | 56-80 | |
| HP1198 | DNA-directed RNA | 21-33, 36-42, 49-60, 68-76, 91-105, 123-130, 141-161, | A: 26, B: 14, | 213-344, | 101, 279 |
| | polymerase, beta | 169-178, 185-190, 192-199, 205-214, 223-233, 239-247, | C: 25, D: 3, | 954-1080, | |
| | subunit (rpoB) | 260-269, 284-293, 300-314, 324-352, 357-364, 373-382, | F: 1, G: 3, I: | 2524-2733 | |
| | | 389-403, 420-432, 438-446, 466-471, 477-484, 503-509, | 3, J: 31, K: 2 | | |
| | | 549-556, 558-576, 600-623, 625-635, 654-661, 663-669, | | | |
| | | 671-687, 702-716, 735-741, 744-750, 757-766, 776-786, | | | |
| | | 807-815, 824-832, 854-860, 863-897, 909-915, 920-946, | | | |
| | | 952-959, 982-997, 1024-1038, 1049-1055, 1071-1085, | | | |
| | | 1104-1113, 1121-1132, 1138-1150, 1187-1196, | | | |
| | | 1212-1221, 1227-1236, 1257-1262, 1264-1278, | | | |
| | | 1282-1294, 1307-1318, 1353-1370, 1382-1388, | | | |
| | | 1396-1409, 1434-1440, 1446-1454, 1465-1478, | | | |
| | | 1485-1513, 1516-1529, 1540-1545, 1563-1568, | | | |
| | | 1575-1593, 1607-1616, 1628-1645, 1648-1661, | | | |
| | | 1676-1682, 1689-1697, 1713-1719, 1739-1749, | | | |
| | | 1753-1758, 1763-1774, 1797-1803, 1807-1846, | | | |
| | | 1855-1874, 1877-1891, 1893-1907, 1912-1925, | | | |
| | | 1931-1943, 1955-1965, 1976-1990, 2032-2043, | | | |
| | | 2045-2051, 2099-2105, 2131-2138, 2161-2179, | | | |
| | | 2188-2199, 2205-2216, 2219-2227, 2235-2245, | | | |
| | | 2247-2267, 2277-2288, 2294-2304, 2314-2326, | | | |
| | | 2346-2358, 2365-2377, 2383-2402, 2407-2423, | | | |
| | | 2437-2450, 2454-2473, 2489-2497, 2525-2531, | | | |
| | | 2557-2570, 2580-2587, 2589-2599, 2621-2641, | | | |
| | | 2647-2653, 2661-2677, 2685-2690, 2697-2717, | | | |
| | | 2722-2733, 2739-2777, 2786-2793, 2801-2808, | | | |
| | | 2811-2822, 2825-2835, 2838-2845, 2859-2871, | | | |
| | | 2877-2883 | | | |
| HP1205 | translation | 10-16, 18-23, 28-41, 63-69, 77-91, 101-109, 118-136, | A: 4, B: 1 | 178-328 | 102, 280 |
| | elongation factor | 146-153, 155-162, 168-179, 192-207, 217-226, 229-235, | | | |
| | EF-Tu (tufB) | 239-254, 279-286, 294-307, 313-319, 334-341, 344-353, | | | |
| | | 363-377, 390-396 | | | |
| HP1229 | aspartokinase (lysC) | 18-42, 68-84, 89-95, 100-105, 107-115, 125-135, 154-177, | I: 1, K: 1, L: 3 | 85-97 | 103, 281 |
| | | 189-195, 205-228, 236-243, 252-259, 279-300, 309-316, | | | |
| | | 323-331, 340-351, 353-364, 377-402 | | | |
| HP1243 | outer membrane | 4-18, 26-32, 66-76, 100-126, 151-159, 178-186, 188-194, | B: 3, F: 1, G: | 21-200, | 104, 282 |
| | protein (omp28) | 200-210, 241-248, 253-259, 262-279, 284-291, 307-313, | 1, H: 1 | 468-480 | |
| | | 315-322, 327-337, 376-386, 399-407, 432-441, 467-473, | | | |
| | | 487-497, 499-505, 543-549, 560-568, 585-593, 598-604, | | | |
| | | 608-614, 630-642, 647-653, 690-703, 717-730 | | | |
| HP1254 | biotin synthesis | 17-49, 52-58, 62-73, 78-97, 100-117, 122-172, 185-190, | H: 2, I: 2 | 33-42 | 105, 283 |
| | protein (bioC) | 193-217, 225-236 | | | |
| HP1265 | hypothetical protein | 7-39, 50-58, 73-89, 96-107, 109-120, 126-142, 152-170, | F: 2, G: 6, I: 1 | 158-169 | 106, 284 |
| | | 178-202, 205-211, 224-244, 249-259, 261-270, 300-310, | | | |
| | | 312-325 | | | |
| HP1282 | anthranilate | 4-31, 40-64, 71-82, 85-92, 102-124, 126-139, 147-152, | E: 20, G: 1, | 201-221 | 107, 285 |
| | synthase component | 159-173, 176-188, 195-207, 210-216, 234-241, 249-256, | I: 1 | | |
| | I (trpE) | 258-276, 279-293, 296-302, 310-315, 349-356, 363-378, | | | |
| | | 380-403, 411-426, 435-441, 448-459, 463-476, 488-494 | | | |
| HP1329 | cation efflux system | 5-13, 15-74, 87-104, 107-120, 123-129, 136-145, 150-191, | B: 2, F: 1, J: | 236-259 | 108, 286 |
| | protein (czcA) | 193-206, 227-248, 250-264, 278-302, 304-323, 332-378, | 1, L: 2 | | |
| | | 384-407, 409-419, 425-457, 462-471, 474-497, 511-545, | | | |
| | | 555-564, 571-578, 585-598, 640-647, 669-675, 682-691, | | | |
| | | 693-705, 729-743, 752-761, 772-780, 786-804, 808-818, | | | |
| | | 822-846, 858-880, 884-900, 910-939, 941-947, 962-971, | | | |
| | | 973-988, 998-1003, 1007-1027 | | | |
| HP1339 | biopolymer | 4-19, 27-68, 81-111, 121-160 | F: 5, I: 4 | 60-79 | 109, 287 |
| | transport protein | | | | |
| | (exbB) | | | | |
| HP1341 | siderophore- | 4-37, 40-46, 52-57, 199-205, 222-229, 236-244, 250-267, | A: 20, B: 23, | 27-197 | 110, 288 |
| | mediated iron | 269-282 | C: 30, E: 31, | | |
| | transport protein | | F: 5, G: 3, | | |
| | | | H: 6, I: 1, J: | | |
| | | | 2, K: 5 | | |
| HP1342 | outer membrane | 4-16, 24-30, 32-38, 63-75, 86-92, 98-111, 113-126, | A: 1, B: 3, | 546-559 | 111, 289 |
| | protein (omp29) | 160-165, 170-180, 198-204, 227-233, 239-245, 253-273, | C: 1, D: 1, | | |
| | | 308-314, 352-365, 382-387, 395-403, 423-429, | E: 1, F: 2, | | |
| | | 472-482, 484-493, 501-507, 518-526, 536-541, 543-550, | H: 7 | | |
| | | 556-562, 586-600, 626-633, 649-661, 680-688 | | | |
| HP1345 | phosphoglycerate | 16-33, 48-59, 63-71, 77-92, 94-109, 117-124, 139-151, | D: 20, I: 6 | 258-272 | 112, 290 |
| | kinase | 169-181, 184-227, 233-249, 251-261, 263-275, 282-294, | | | |
| | | 297-321, 326-332, 341-355, 383-399 | | | |
| HP1350 | protease | 11-26, 31-39, 43-52, 55-62, 64-70, 80-94, 123-133, 135-141, | A: 3, B: 1, | 77-226, | 113, 291 |
| | | 172-181, 185-206, 209-218, 224-230, 238-244, 251-262, | C: 1, G: 1, | 350-429 | |
| | | 264-271, 290-301, 306-324, 333-340, 350-357, 367-375, | K: 2, M: 12 | | |
| | | 390-397, 434-441, 443-448 | | | |
| HP1374 | ATP-dependent | 4-13, 22-27, 31-45, 50-59, 72-96, 99-114, 131-141, 143-150, | G: 9, M: 2 | 283-305 | 114, 292 |
| | protease ATPase | 159-176, 180-186, 189-198, 208-214, 234-253, 271-287, | | | |
| | subunit (clpX) | 294-299, 310-366, 382-390, 398-416, 424-443 | | | |
| HP1393 | DNA repair protein | 9-26, 30-53, 62-72, 86-95, 112-122, 136-145, 153-160, | E: 1, F: 2, G: | 213-232 | 115, 293 |
| | (recN) | 209-221, 227-237, 241-268, 281-288, 291-298, 308-314, | 7, I: 1 | | |
| | | 321-328, 336-346, 351-379, 388-397, 409-416, 423-433, | | | |
| | | 443-481, 511-519 | | | |
| HP1448 | ribonuclease P, | 12-18, 25-31, 38-50, 59-67, 71-82, 96-126 | G: 6 | 76-88 | 116, 294 |
| | protein component | | | | |
| | (rnpA) | | | | |
| HP1453 | conserved | 4-25, 39-44, 64-71, 74-88, 100-113, 128-138, 151-162, | B: 1, D: 4, | 2-100 | 117, 295 |
| | hypothetical protein | 164-177, 185-190, 204-213, 233-239, 246-254, 281-286, | F: 2, J: 1, K: | | |
| | | 293-306, 309-318, 333-347, 349-359, 385-398, 404-423, | 11, L: 3 | | |
| | | 458-465, 477-484, 490-499, 501-533, 554-566, 582-590, | | | |
| | | 596-616, 624-629, 631-639, 654-680, 694-720, 735-743 | | | |
| HP1454 | hypothetical protein | 4-16, 36-41, 52-75, 98-107, 109-117, 122-128, 133-139, | B: 1, M: 8 | 75-92 | 118, 296 |
| | | 141-155, 159-165, 169-182, 187-193, 195-201, 211-224, | | | |
| | | 230-236, 247-269, 278-290 | | | |
| HP1460 | DNA polymerase III | 7-21, 25-33, 37-43, 87-94, 103-120, 131-147, 168-174, | F: 3, G: 2, I: 3 | 732-748 | 119, 297 |
| | alpha-subunit | 197-203, 207-212, 227-237, 247-257, 263-271, 279-287, | | | |
| | (dnaE) | 298-306, 320-325, 332-340, 363-374, 379-384, 390-401, | | | |
| | | 403-414, 428-433, 448-457, 462-475, 483-490, 513-519, | | | |
| | | 525-535, 543-554, 559-566, 571-620, 625-631, 636-642, | | | |
| | | 659-670, 688-706, 708-723, 770-779, 787-793, 796-807, | | | |
| | | 820-840, 848-854, 863-874, 895-905, 912-919, 934-942, | | | |
| | | 968-975, 983-1000, 1012-1019, 1026-1036, 1050-1060, | | | |
| | | 1064-1070, 1081-1091, 1094-1108, 1112-1118, | | | |
| | | 1140-1152, 1164-1169, 1172-1180, 1187-1192 | | | |
| HP1497 | peptidyl-tRNA | 23-40, 42-59, 66-73, 78-97, 111-128, 130-141, 157-166, | E: 6, F: 1, | 53-71 | 120, 298 |
| | hydrolase (pth) | 178-183 | H: 2 | | |
| HP1527 | hypothetical protein | 4-27, 38-44, 47-57, 59-85, 99-106, 114-121, 154-166, | B: 3, C: 1, I: 7 | 86-195 | 121, 299 |
| | | 181-186, 193-198, 238-244, 253-262, 272-278, 287-299, | | | |
| | | 314-320, 338-350, 358-368, 382-388, 407-416, 433-446, | | | |
| | | 456-461, 463-473 | | | |
| HP1564 | outer membrane | 5-24, 38-59, 64-80, 87-99, 105-126, 134-142, 149-163, | A: 2, B: 1, | 87-245 | 122, 300 |
| | protein | 165-179, 181-202, 205-220, 227-233, 243-250, 257-263 | C: 3, F: 1, | | |
| | | | H: 1 | | |
| HP1565 | pencillin-binding | 5-32, 47-53, 66-79, 81-97, 115-151, 155-174, 183-188, | A: 1, F: 2, | 347-371, | 123, 301 |
| | protein 2 (pbp2) | 196-210, 215-226, 230-238, 253-258, 263-270, 276-282, | H: 1, L: 5 | 375-386 | |
| | | 295-301, 304-325, 334-344, 360-390, 397-412, 425-432, | | | |
| | | 434-462, 478-494, 508-526, 539-564, 571-579 | | | |
| HP1574 | riboflavin synthase | 4-15, 36-44, 49-56, 60-66, 68-82, 84-103, 109-115, 118-141, | H: 1, M: 5 | 26-39 | 124, 302 |
| | alpha subunit (ribC) | 147-154, 160-168, 176-185 | | | |
| ARF0044 | Hypothetical | 7-13, 23-33 | H: 1, I: 2 | 13-21 | 125, 303 |
| | protein | | | | |
| ARF0048 | Hypothetical | none | F: 1, G: 1, I: | 2-10 | 126, 304 |
| | protein | | 4, L: 8 | | |
| ARF0143 | Hypothetical | 4-9, 12-18, 35-42, 49-62 | F: 1 | 6-18 | 127, 305 |
| | protein | | | | |
| ARF0184 | Hypothetical | 19-25 | A: 1, I: 6 | 1-13 | 128, 306 |
| | protein | | | | |
| ARF0219 | Hypothetical | 15-21, 27-45 | E: 7 | 12-25 | 129, 307 |
| | protein | | | | |
| ARF0308 | Hypothetical | 14-20 | E: 41 | 1-14 | 130, 308 |
| | protein | | | | |
| ARF0349 | Hypothetical | 4-18 | G: 11 | 13-26 | 131, 309 |
| | protein | | | | |
| ARF0387 | Hypothetical | 8-21 | G: 12, H: 2, | 2-20 | 132, 310 |
| | protein | | K: 1 | | |
| ARF0402 | Hypothetical | 4-14 | F: 2, G: 10, | 4-16 | 133, 311 |
| | protein | | H: 1, M: 2 | | |
| ARF0501 | Hypothetical | none | M: 2 | 3-12 | 134, 312 |
| | protein | | | | |
| ARF0509 | Hypothetical | 6-14, 6-25, 35-57 | G: 5, H: 1, | 2-14 | 135, 313 |
| | protein | | M: 3 | | |
| ARF0522 | Hypothetical | 6-25, 35-57 | F: 1, I: 3, K: 2 | 17-31 | 136, 314 |
| | protein | | | | |
| ARF0578 | Hypothetical | 14-25, 32-46 | E: 12, G: 1 | 5-19 | 137, 315 |
| | protein | | | | |
| ARF0629 | Hypothetical | 18-31 | G: 1, I: 7 | 5-16 | 138, 316 |
| | protein | | | | |
| ARF0665 | Hypothetical | 19-24 | L: 2, M: 5 | 4-26 | 139, 317 |
| | protein | | | | |
| ARF0693 | Hypothetical | 13-21, 29-34, 47-58, 61-73 | G: 1, I: 6 | 36-47 | 140, 318 |
| | protein | | | | |
| ARF0752 | Hypothetical | 4-15 | D: 3 | 5-24 | 141, 319 |
| | protein | | | | |
| ARF0788 | Hypothetical | none | B: 1, H: 1 | 6-18 | 142, 320 |
| | protein | | | | |
| ARF0819 | Hypothetical | 13-20 | F: 14 | 4-13 | 143, 321 |
| | protein | | | | |
| ARF0839 | Hypothetical | none | A: 2, C: 1, I: | 15-23 | 144, 322 |
| | protein | | 1, K: 3 | | |
| ARF0868 | Hypothetical | 4-9 | F: 2, G: 1 | 7-21 | 145, 323 |
| | protein | | | | |
| ARF0948 | Hypothetical | none | F: 1 | 1-10 | 146, 324 |
| | protein | | | | |
| ARF0969 | Hypothetical | none | B: 1, G: 1, | 4-14 | 147, 325 |
| | protein | | M: 2 | | |
| ARF1100 | Hypothetical | 4-17, 35-41, 46-89, 93-98 | A: 1, B: 1, | 70-88 | 148, 326 |
| | protein | | H: 1, I: 2, | | |
| | | | M: 5 | | |
| ARF1164 | Hypothetical | none | G: 1, H: 3, J: | 1-13 | 149, 327 |
| | protein | | 1, M: 2 | | |
| ARF1470 | Hypothetical | 4-16, 26-32 | G: 1, I: 1, | 25-38 | 150, 328 |
| | protein | | M: 2 | | |
| ARF1553 | Hypothetical | 8-15, 23-28 | B: 2, F: 6, G: | 4-17 | 151, 329 |
| | protein | | 2, H: 1, I: 1, | | |
| | | | K: 1 | | |
| CRF0017 | Hypothetical | 4-12 | H: 1, I: 1, | 1-15 | 152, 330 |
| | protein | | M: 5 | | |
| CRF0025 | Hypothetical | 4-29, 31-42, 52-58 | E: 1, G: 1, | 6-16 | 153, 331 |
| | protein | | L: 3, M: 1 | | |
| CRF0090 | Hypothetical | 4-9, 24-32 | F: 13, G: 1, | 9-19 | 154, 332 |
| | protein | | H: 1, L: 1 | | |
| CRF0127 | Hypothetical | 4-12, 18-27 | L: 3, M: 10 | 5-18 | 155, 333 |
| | protein | | | | |
| CRF0169 | Hypothetical | 4-11, 37-56, 58-92 | D: 3 | 18-29 | 156, 334 |
| | protein | | | | |
| CRF0190 | Hypothetical | 8-28 | M: 14 | 20-35 | 157, 335 |
| | protein | | | | |
| CRF0251 | Hypothetical | none | D: 16, E: 2, | 4-15 | 158, 336 |
| | protein | | G: 3 | | |
| CRF0258 | Hypothetical | 4-23, 27-39, 55-63 | A: 1, B: 1, | 35-58 | 159, 337 |
| | protein | | C: 1, F: 1, L: 5 | | |
| CRF0354 | Hypothetical | 6-26, 28-54 | F: 8, H: 1 | 28-47 | 160, 338 |
| | protein | | | | |
| CRF0388 | Hypothetical | 4-10, 38-52, 58-82 | H: 1, L: 3 | 30-49 | 161, 339 |
| | protein | | | | |
| CRF0409 | Hypothetical | 4-22, 29-35, 44-50, 53-68, 70-80 | G: 2, H: 1, | 20-33 | 162, 340 |
| | protein | | M: 10 | | |
| CRF0421 | Hypothetical | 22-28, 30-36 | F: 4 | 18-33 | 163, 341 |
| | protein | | | | |
| CRF0480 | Hypothetical | 4-11, 13-21, 25-30 | E: 1, L: 8 | 20-30 | 164, 342 |
| | protein | | | | |
| CRF0552 | Hypothetical | 10-22 | G: 1, M: 5 | 10-23 | 165, 343 |
| | protein | | | | |
| CRF0563 | Hypothetical | 4-11 | G: 1, L: 1, | 9-20 | 166, 344 |
| | protein | | M: 6 | | |
| CRF0578 | Hypothetical | 14-25, 32-46 | G: 1, L: 7, | 6-19 | 167, 345 |
| | protein | | M: 12 | | |
| CRF0626 | Hypothetical | 5-30 | L: 43 | 14-33 | 168, 346 |
| | protein | | | | |
| CRF0870 | Hypothetical | 4-15, 28-35, 46-55, 59-65, 76-84 | H: 6, I: 1, L: 8 | 9-24 | 169, 347 |
| | protein | | | | |
| CRF0894 | Hypothetical | 27-33 | L: 5 | 5-19 | 170, 348 |
| | protein | | | | |
| CRF0922 | Hypothetical | 5-13 | E: 11, F: 3, | 8-18 | 171, 349 |
| | protein | | G: 1, H: 7, I: | | |
| | | | 1, L: 4 | | |
| CRF1012 | Hypothetical | 9-22, 24-34 | L: 3 | 21-40 | 172, 350 |
| | protein | | | | |
| CRF1100 | Hypothetical | 4-17, 35-41, 46-89, 93-98 | E: 1, H: 5, I: | 71-89 | 173, 351 |
| | protein | | 2, L: 4 | | |
| CRF1301 | Hypothetical | 4-12, 14-24 | H: 9, I: 2 | 2-17 | 174, 352 |
| | protein | | | | |
| CRF1354 | Hypothetical | 9-17 | I: 2, M: 16 | 5-16 | 175, 353 |
| | protein | | | | |
| CRF1422 | Hypothetical | 7-41, 48-58, 63-75, 80-89 | G: 2, H: 1, | 43-53 | 176, 354 |
| | protein | | L: 62 | | |
| CRF1489 | Hypothetical | 4-22, 25-30 | E: 1, F: 1, | 4-14 | 177, 355 |
| | protein | | H: 5, L: 10 | | |
| CRF1549 | Hypothetical | 4-55 | G: 1, M: 7 | 18-33 | 178, 356 |
| | protein | | | | |

TABLE 2
Gene distribution in H. pylori strains.

| H. pylori antigenic | Putative function | Gene distribution | Amino acid substitutions (in | Seq. ID |
|---|---|---|---|---|
| protein | (by homology) | (presence in 28 strains) | cancer patient isolate)* | (DNA, Prot.) |
| HP0009 | outer membrane protein (omp1) | n.d. | n.d. | 1, 179 |
| HP0010 | chaperone and heat shock | n.d. | n.d. | 2, 180 |
| | protein (groEL) | | | |
| HP0043 | mannose-6-phosphate isomerase | 28/28 | 11/247 | 3, 181 |
| | (pmi) | | | |
| HP0063 | hypothetical protein | n.d. | n.d. | 4, 182 |
| HP0067 | urease accessory protein (ureH) | 13/28 | 9/230 | 5, 183 |
| HP0072 | urease beta subunit (urea | n.d. | n.d. | 6, 184 |
| | amidohydrolase) (ureB) | | | |
| HP0086 | Conserved hypothetical protein | 28/28 | 1/221 | 7, 185 |
| HP0087 | hypothetical protein | 28/28 | 15/241 | 8, 186 |
| HP0088 | RNA polymerase sigma-70 factor | 28/28 | 4/226 | 9, 187 |
| | (rpoD) | | | |
| HP0089 | pfs protein (pfs) | 28/28 | 5/199 | 10, 188 |
| HP0115 | flagellin B (flaB) | 28/28 | 2/249 | 11, 189 |
| HP0175 | cell binding factor 2 | n.d. | n.d. | 12, 190 |
| HP0121 | phosphoenolpyruvate synthase | 28/28 | 0/240 | 13, 191 |
| | (ppsA) | | | |
| HP0123 | threonyl-tRNA synthetase (thrS) | 28/28 | 7/238 | 14, 192 |
| HP0130 | hypothetical protein | 28/28 | 8/241 | 15, 193 |
| HP0150 | hypothetical protein | 28/28 | 5/111 | 16, 194 |
| HP0183 | serine hydroxymethyltransferase | 28/28 | 2/227 | 17, 195 |
| | (glyA) | | | |
| HP0192 | fumarate reductase, flavoprotein | 28/28 | 3/219 | 18, 196 |
| | subunit (frdA) | | | |
| HP0197 | S-adenosylmethionine | 28/28 | 1/243 | 19, 197 |
| | synthetase 2 (metX) | | | |
| HP0201 | fatty acid/phospholipid synthesis | 28/28 | 0/176 | 20, 198 |
| | protein (plsX) | | | |
| HP0202 | beta-ketoacyl-acyl carrier protein | 28/28 | 7/232 | 21, 199 |
| | synthase III | | | |
| HP0210 | chaperone and heat shock | n.d. | n.d. | 22, 200 |
| | protein C62.5 (htpG) | | | |
| HP0211 | conserved hypothetical secreted | 28/28 | 3/201 | 23, 201 |
| | protein | | | |
| HP0228 | conserved hypothetical integral | 28/28 | 3/248 | 24, 202 |
| | membrane protein | | | |
| HP0229 | outer membrane protein (omp6) | n.d. | n.d. | 25, 203 |
| HP0235 | conserved hypothetical secreted | n.d. | n.d. | 26, 204 |
| | protein | | | |
| HP0239 | glutamyl-tRNA reductase | 7/28 | 4/257^{#} | 27, 205 |
| | (hemA) | | | |
| HP0258 | conserved hypothetical integral | 28/28 | 14/254 | 28, 206 |
| | membrane protein | | | |
| HP0266 | dihydroorotase (pyrC) | 15/28 | 14/253 | 29, 207 |
| HP0279 | lipopolysaccharide | 28/28 | 15/246 | 30, 208 |
| | heptosyltransferase-1 (rfaC) | | | |
| HP0289 | toxin-like outer membrane | 28/28 | 12/241 | 31, 209 |
| | protein | | | |
| HP0292 | hypothetical protein | 28/28 | 9/245 | 32, 210 |
| HP0295 | flagellin B homolog (fla) | 14/28 | 9/224^{#} | 33, 211 |
| HP0349 | CTP synthetase (pyrG) | 28/28 | 2/249 | 34, 212 |
| HP0351 | flagellar basal-body M-ring | 28/28 | 2/249 | 35, 213 |
| | protein (fliF) | | | |
| HP0380 | glutamate dehydrogenase | 28/28 | 9/253 | 36, 214 |
| | (gdhA) | | | |
| HP0392 | histidine kinase (cheA) | 27/28 | 4/259; 5 aa inserted, 4 aa | 37, 215 |
| | | | deleted | |
| HP0401 | 3-phosphoshikimate 1- | 28/28 | 14/248 | 38, 216 |
| | carboxyvinyltransferase (aroA) | | | |
| HP0406 | hypothetical protein | 28/28 | 2/146 | 39, 217 |
| HP0409 | GMP synthase (guaA) | 28/28 | 9/252 | 40, 218 |
| HP0413 | transposase-like protein, PS3IS | 10/28 | 0/268^{#} | 41, 219 |
| HP0459 | virB4 homolog (virB4) | 9/28 | 6/248^{#} | 42, 220 |
| HP0480 | GTP-binding protein, fusA- | n.d. | n.d. | 43, 221 |
| | homolog (yihK) | | | |
| HP0485 | catalase-like protein | 28/28 | 7/252 | 44, 222 |
| HP0508 | hypothetical protein | 28/28 | 3/260 | 45, 223 |
| HP0519 | conserved hypothetical protein | 28/28 | 6/224; 1 aa inserted, 1 aa | 46, 224 |
| | | | deleted | |
| HP0525 | vitB11 homolog | 24/28 | 0/257 | 47, 225 |
| HP0527 | cag pathogenicity island protein | 22/28 | 4/214 | 48, 226 |
| | (cag7-cagY) | | | |
| HP0540 | cag pathogenicity island protein | 25/28 | 5/251 | 49, 227 |
| | (cag19) | | | |
| HP0541 | cag pathogenicity island protein | 26/28 | 3/251 | 50, 228 |
| | (cag20) | | | |
| HP0542 | cag pathogenicity island protein | 23/28 | 0/115 | 51, 229 |
| | (cag21-cagG) | | | |
| HP0544 | cag pathogenicity island protein | 11/28 | n.d. | 52, 230 |
| | (cag23-cagE) | | | |
| HP0545 | cag pathogenicity island protein | 25/28 | 1/153 | 53, 231 |
| | (cag24) | | | |
| HP547 | cag pathogenicity island protein | 24/28 | 20/256; 1 aa inserted^{#} | 54, 232 |
| | (cag26-cagA) | | | |
| HP0563 | hypothetical protein | 28/28 | 9/192 | 55, 233 |
| HP0604 | uroporphyrinogen | 28/28 | 5/260 | 56, 234 |
| | decarboxylase (hemE) | | | |
| HP0607 | acriflavine resistance protein | 28/28 | 0/254 | 57, 235 |
| | (acrB) | | | |
| HP0630 | modulator of drug activity | 28/28 | 2/112^{#} | 58, 236 |
| | (mda66) | | | |
| HP0635 | hypothetical protein | 24/28 | 13/135 | 59, 237 |
| HP0655 | protective surface antigen D15 | 28/28 | n.d. | 60, 238 |
| HP0659 | hypothetical protein | 6/28 | 14/187 | 61, 239 |
| HP0683 | UDP-N-acetylglucosamine | 28/28 | 8/193 | 62, 240 |
| | pyrophosphorylase (glmU) | | | |
| HP0687 | iron(II) transport protein (feoB) | 28/28 | 2/203 | 63, 241 |
| HP0696 | N-methylhydantoinase | 28/28 | 2/206 | 64, 242 |
| HP0701 | DNA gyrase, sub A (gyrA) | 28/28 | 5/224 | 65, 243 |
| HP0706 | outer membrane protein (omp15) | 28/28 | 2/167 | 66, 244 |
| HP0714 | RNA polymerase sigma-54 factor | 28/28 | 8/200 | 67, 245 |
| | (rpoN) | | | |
| HP0717 | DNA polymerase III gamma and | 28/28 | 15/137; 2 aa inserted^{#} | 68, 246 |
| | tau subunits (dnaX) | | | |
| HP0723 | L-asparaginase II (ansB) | 28/28 | 12/220 | 69, 247 |
| HP0727 | transcriptional regulator, | 28/28 | 4/207 | 70, 248 |
| | putative | | | |
| HP0752 | flagellar hook-associated protein | 28/28 | 2/191 | 71, 249 |
| | 2 (fliD) | | | |
| HP0760 | conserved hypothetical protein | 28/28 | 2/211 | 72, 250 |
| HP0836 | hypothetical protein | 28/28 | 1/82 | 73, 251 |
| HP0850 | type I restriction enzyme M | 28/28 | 10/181 | 74, 252 |
| | protein (hsdM) | | | |
| HP0853 | ABC transporter, ATP-binding | 19/28 | 2/198^{#} | 75, 253 |
| | protein (yheS) | | | |
| HP0863 | hypothetical protein | 27/28 | 2/161 | 76, 254 |
| HP0874 | hypothetical protein | 28/28 | 3/243 | 77, 255 |
| HP0875 | catalase | n.d. | n.d. | 78, 256 |
| HP0876 | iron-regulated outer membrane | 28/28 | 4/193 | 79, 257 |
| | protein (frpB) | | | |
| HP0887 | vacuolating cytotoxin | 28/28 | 9/228^{#} | 80, 258 |
| HP0891 | conserved hypothetical protein | 18/28 | 2/149^{#} | 81, 259 |
| HP0910 | adenine specific DNA | 27/28 | 3/205 | 82, 260 |
| | methyltransferase (HINDIIM) | | | |
| HP0913 | outer membrane protein (omp21) | 28/28 | 12/172; 1 aa deleted | 83, 261 |
| HP0922 | toxin-like outer membrane | 28/28 | 27/198 | 84, 262 |
| | protein | | | |
| HP0925 | recombinational DNA repair | 28/28 | 1/159 | 85, 263 |
| | protein (recR) | | | |
| HP0953 | hypothetical protein | 28/28 | 2/164 | 86, 264 |
| HP0973 | hypothetical protein | 19/28 | 6/248; 1 aa deleted | 87, 265 |
| HP0977 | conserved hypothetical secreted | 28/28 | 9/238 | 88, 266 |
| | protein | | | |
| HP1019 | serine protease (htrA) | n.d. | n.d. | 89, 267 |
| HP1024 | co-chaperone-curved DNA | 25/28 | 5/167 | 90, 268 |
| | binding protein A (CbpA) | | | |
| HP1052 | UDP-3-0-acyl N- | 28/28 | 5/186 | 91, 269 |
| | acetylglcosamine deacetylase | | | |
| | (envA) | | | |
| HP1090 | cell division protein (ftsK) | 28/28 | 2/223 | 92, 270 |
| HP1098 | conserved hypothetical secreted | n.d. | n.d. | 93, 271 |
| | protein | | | |
| HP1116 | hypothetical protein | 28/28 | 192/283 | 94, 272 |
| HP1117 | conserved hypothetical secreted | n.d. | n.d. | 95, 273 |
| | protein | | | |
| HP1119 | flagellar hook-associated protein | 28/28 | 7/213 | 96, 274 |
| | 1 (HAP1) (flgK) | | | |
| HP1126 | colicin tolerance-like protein | 28/28 | 4/241 | 97, 275 |
| | (tolB) | | | |
| HP1152 | signal recognition particle | n.d. | n.d. | 98, 276 |
| | protein (ffh) | | | |
| HP1153 | valyl-tRNA synthetase (valS) | 28/28 | 14/243 | 99, 277 |
| HP1186 | carbonic anhydrase | | | 100, 278 |
| HP1198 | DNA-directed RNA polymerase, | 28/28 | 7/232 | 101, 279 |
| | beta subunit (rpoB) | | | |
| HP1205 | translation elongation factor EF- | n.d. | n.d. | 102, 280 |
| | Tu (tufB) | | | |
| HP1229 | aspartokinase (lysC) | 2728 | 4/245^{#} | 103, 281 |
| HP1243 | outer membrane protein (omp28) | n.d. | n.d. | 104, 282 |
| HP1254 | biotin synthesis protein (bioC) | 28/28 | 9/169 | 105, 283 |
| HP1265 | hypothetical protein | 28/28 | 19/216 | 106, 284 |
| HP1282 | anthranilate synthase component | 28/28 | 12/193 | 107, 285 |
| | I (trpE) | | | |
| HP1329 | cation efflux system protein | 28/28 | 3/196 | 108, 286 |
| | (czcA) | | | |
| HP1339 | biopolymer transport protein | 27/28 | 1/109^{#} | 109, 287 |
| | (exbB) | | | |
| HP1341 | siderophore-mediated iron | 28/28 | 12/179^{#} | 110, 288 |
| | transport protein | | | |
| HP1342 | outer membrane protein (omp29) | n.d. | n.d. | 111, 289 |
| HP1345 | phosphoglycerate kinase | 28/28 | 3/220 | 112, 290 |
| HP1350 | protease | n.d. | n.d. | 113, 291 |
| HP1374 | ATP-dependent protease ATPase | 28/28 | 0/211 | 114, 292 |
| | subunit (clpX) | | | |
| HP1393 | DNA repair protein (recN) | 28/28 | 4/209 | 115, 293 |
| HP1448 | ribonuclease P, protein | 19/28 | 5/124^{#} | 116, 294 |
| | component (rnpA) | | | |
| HP1453 | conserved hypothetical protein | 28/28 | 8/200 | 117, 295 |
| HP1454 | hypothetical protein | n.d. | n.d. | 118, 296 |
| HP1460 | DNA polymerase III alpha- | 28/28 | 2/225 | 119, 297 |
| | subunit (dnaE) | | | |
| HP1497 | peptidyl-tRNA hydrolase (pth) | 28/28 | 4/155 | 120, 298 |
| HP1527 | hypothetical protein | 28/28 | 14/202 | 121, 299 |
| HP1564 | outer membrane protein | n.d. | n.d. | 122, 300 |
| HP1565 | penicillin-binding protein 2 | 28/28 | 3/178 | 123, 301 |
| | (pbp2) | | | |
| HP1574 | riboflavin synthase alpha | 23/28 | 4/153^{#} | 124, 302 |
| | subunit (ribC) | | | |
| ARF0044 | Hypothetical protein | n.d. | n.d. | 125, 303 |
| ARF0048 | Hypothetical protein | n.d. | n.d. | 126, 304 |
| ARF0143 | Hypothetical protein | n.d. | n.d. | 127, 305 |
| ARF0184 | Hypothetical protein | n.d. | n.d. | 128, 306 |
| ARF0219 | Hypothetical protein | n.d. | n.d. | 129, 307 |
| ARF0308 | Hypothetical protein | n.d. | n.d. | 130, 308 |
| ARF0349 | Hypothetical protein | n.d. | n.d. | 131, 309 |
| ARF0387 | Hypothetical protein | n.d. | n.d. | 132, 310 |
| ARF0402 | Hypothetical protein | n.d. | n.d. | 133, 311 |
| ARF0501 | Hypothetical protein | n.d. | n.d. | 134, 312 |
| ARF0509 | Hypothetical protein | n.d. | n.d. | 135, 313 |
| ARF0522 | Hypothetical protein | n.d. | n.d. | 136, 314 |
| ARF0578 | Hypothetical protein | n.d. | n.d. | 137, 315 |
| ARF0629 | Hypothetical protein | n.d. | n.d. | 138, 316 |
| ARF0665 | Hypothetical protein | n.d. | n.d. | 139, 317 |
| ARF0693 | Hypothetical protein | n.d. | n.d. | 140, 318 |
| ARF0752 | Hypothetical protein | n.d. | n.d. | 141, 319 |
| ARF0788 | Hypothetical protein | n.d. | n.d. | 142, 320 |
| ARF0819 | Hypothetical protein | n.d. | n.d. | 143, 321 |
| ARF0839 | Hypothetical protein | n.d. | n.d. | 144, 322 |
| ARF0868 | Hypothetical protein | n.d. | n.d. | 145, 323 |
| ARF0948 | Hypothetical protein | n.d. | n.d. | 146, 324 |
| ARF0969 | Hypothetical protein | n.d. | n.d. | 147, 325 |
| ARF1100 | Hypothetical protein | n.d. | n.d. | 148, 326 |
| ARF1164 | Hypothetical protein | n.d. | n.d. | 149, 327 |
| ARF1470 | Hypothetical protein | n.d. | n.d. | 150, 328 |
| ARF1553 | Hypothetical protein | n.d. | n.d. | 151, 329 |
| CRF0017 | Hypothetical protein | n.d. | n.d. | 152, 330 |
| CRF0025 | Hypothetical protein | n.d. | n.d. | 153, 331 |
| CRF0090 | Hypothetical protein | n.d. | n.d. | 154, 332 |
| CRF0127 | Hypothetical protein | n.d. | n.d. | 155, 333 |
| CRF0169 | Hypothetical protein | n.d. | n.d. | 156, 334 |
| CRF0190 | Hypothetical protein | n.d. | n.d. | 157, 335 |
| CRF0251 | Hypothetical protein | n.d. | n.d. | 158, 336 |
| CRF0258 | Hypothetical protein | n.d. | n.d. | 159, 337 |
| CRF0354 | Hypothetical protein | n.d. | n.d. | 160, 338 |
| CRF0388 | Hypothetical protein | n.d. | n.d. | 161, 339 |
| CRF0409 | Hypothetical protein | n.d. | n.d. | 162, 340 |
| CRF0421 | Hypothetical protein | n.d. | n.d. | 163, 341 |
| CRF0480 | Hypothetical protein | n.d. | n.d. | 164, 342 |
| CRF0552 | Hypothetical protein | n.d. | n.d. | 165, 343 |
| CRF0563 | Hypothetical protein | n.d. | n.d. | 166, 344 |
| CRF0578 | Hypothetical protein | n.d. | n.d. | 167, 345 |
| CRF0626 | Hypothetical protein | n.d. | n.d. | 168, 346 |
| CRF0870 | Hypothetical protein | n.d. | n.d. | 169, 347 |
| CRF0894 | Hypothetical protein | n.d. | n.d. | 170, 348 |
| CRF0922 | Hypothetical protein | n.d. | n.d. | 171, 349 |
| CRF1012 | Hypothetical protein | n.d. | n.d. | 172, 350 |
| CRF1100 | Hypothetical protein | n.d. | n.d. | 173, 351 |
| CRF1301 | Hypothetical protein | n.d. | n.d. | 174, 352 |
| CRF1354 | Hypothetical protein | n.d. | n.d. | 175, 353 |
| CRF1422 | Hypothetical protein | n.d. | n.d. | 176, 354 |
| CRF1489 | Hypothetical protein | n.d. | n.d. | 177, 355 |
| CRF1549 | Hypothetical protein | n.d. | n.d. | 178, 356 |

TABLE 3

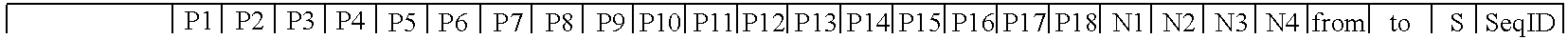

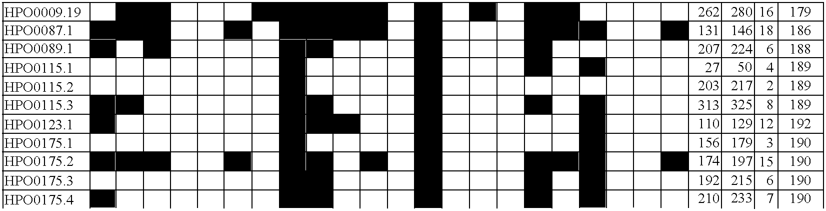

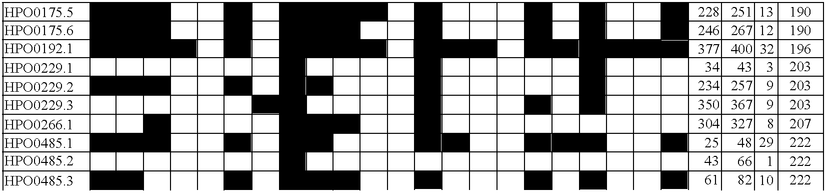

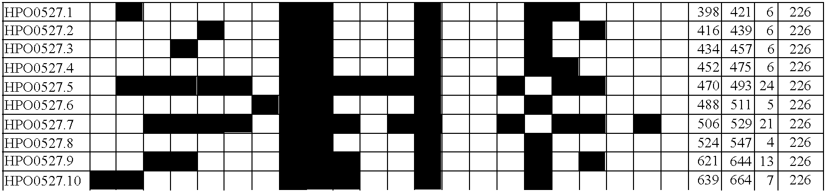

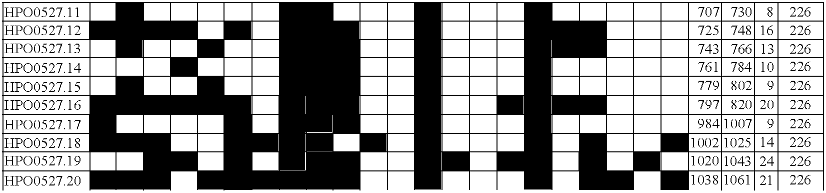

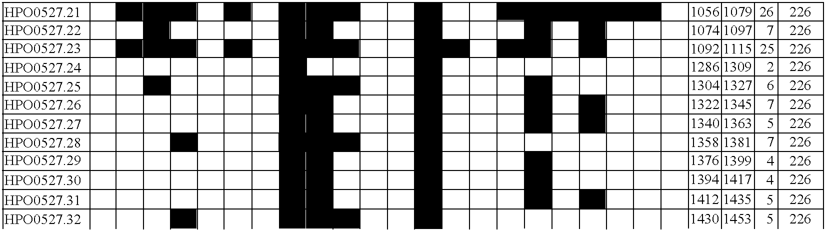

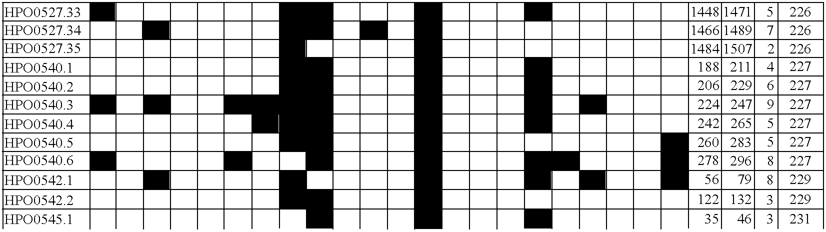

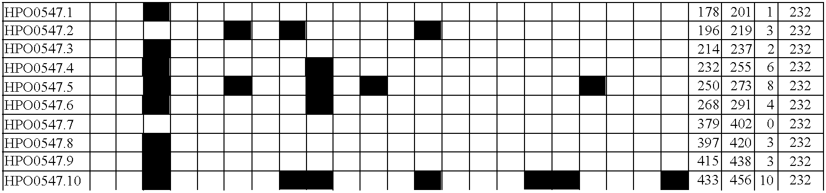

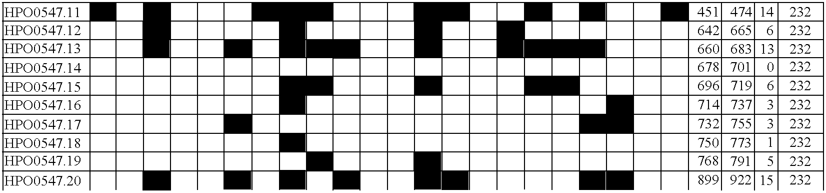

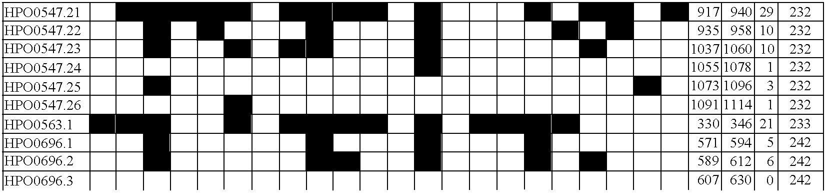

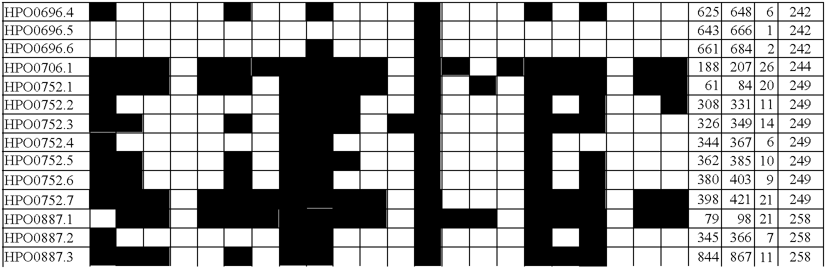

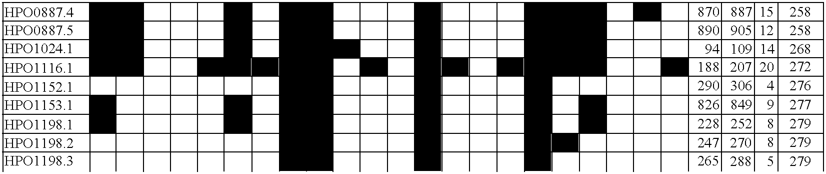

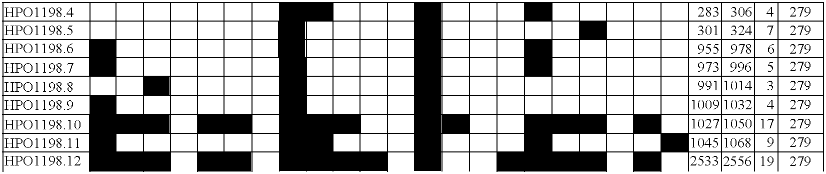

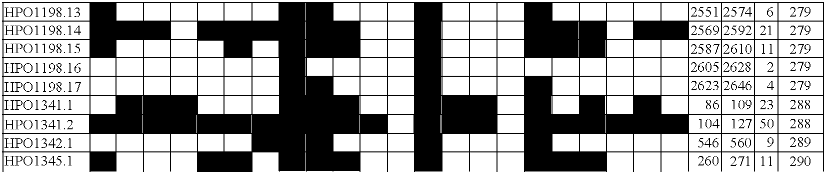

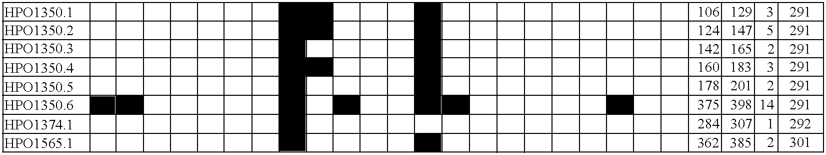

1-37. (canceled)

38. A hyperimmune serum-reactive antigen comprising an amino acid sequence from any of SEQ ID NOs: 179-356 or fragments thereof.

39. The hyperimmune serum-reactive antigen or fragment of claim 38, further defined as a peptide comprising an amino acid sequence described in the “predicted immunogenic aa” and/or “location of identified immunogenic region” of Table 1 or of a serum reactive epitope of Table 3.

40. The hyperimmune serum reactive antigen or fragment of claim 38, further defined as comprising an amino acid sequence of amino acids: 63-91, 95-101, 110-116, 134-148, 150-156, 158-164, 188-193, 197-209, 226-241, 247-254, 291-297, 312-319, 338-346, 351-358, 366-378, 404-410, 420-438, 448-454, 465-473, 482-488, 490-498, 503-510, 512-519, 531-543, 547-554, 568-575, 589-604, 610-631 and 239-308 of SEQ ID NO:179; 16-29, 35-47, 50-68, 70-79, 91-101, 143-149, 158-163, 185-191, 196-206, 215-224, 230-237, 244-251, 258-278, 290-311, 319-325, 338-351, 365-385, 396-429, 445-454, 458-466, 491-499, 501-521, 17-79 and 218-233 of SEQ ID NO:180; 4-10, 16-41, 46-66, 77-84, 91-97, 102-118, 125-144, 187-200, 202-214, 245-253, 255-261, 286-295, 300-330, 335-342, 350-361, 363-381, 385-392, 396-416, 435-450 and 460-470 of SEQ ID NO:181; 11-19, 27-48, 52-59, 77-82, 84-107, 118-125, 127-154, 178-183, 192-209, 215-221, 286-295, 302-313, 350-357, 402-415, 417-431, 453-463, 465-493 and 313-331 of SEQ ID NO:182; 19-26, 30-43, 47-55, 63-68, 72-80, 97-104, 107-119, 129-146, 160-175, 194-216, 231-251, 254-260 and 26-43 of SEQ ID NO:183; 7-13, 29-37, 65-81, 110-120, 123-131, 135-152, 230-249, 254-260, 284-290, 292-299, 317-326, 329-336, 403-444, 452-458, 466-477, 490-498, 510-519, 541-550, 557-566 and 533-567 of SEQ ID NO:184; 5-47, 71-77, 79-86, 89-95, 120-126, 137-144, 176-181, 184-196, 202-208, 211-232, 236-282, 301-313, 317-325, 341-347, 353-384, 394-400, 412-433, 436-443 and 59-75 of SEQ ID NO:185; 4-18, 22-38, 59-69, 106-112, 116-130, 138-149, 156-170, 175-197, 200-214, 216-223, 233-244, 255-261, 266-276, 279-286, 325-333, 342-348, 366-399, 402-420, 429-441, 1-104 and 130-147 of SEQ ID NO:186; 50-58, 69-95, 97-113, 131-136, 157-163, 170-175, 188-212, 220-226, 254-259, 265-277, 283-289, 297-308, 311-318, 347-358, 360-369, 378-401, 416-421, 440-450, 454-462, 470-476, 493-502, 506-514, 536-567, 585-590, 598-607, 613-618, 653-659 and 35-46 of SEQ ID NO:187; 16-29, 32-60, 65-87, 89-123, 128-134, 137-158, 162-173, 178-196, 210-216, 218-228 and 206-225 of SEQ ID NO:188; 10-20, 26-35, 51-64, 86-91, 94-100, 113-122, 154-160, 185-191, 193-201, 211-217, 225-230, 237-246, 251-257, 298-304, 306-312, 316-328, 340-348, 357-389, 391-397, 415-421, 449-456, 458-471, 488-495, 502-511, 24-55 and 236-341 of SEQ ID NO:189; 5-22, 41-51, 87-93, 114-122, 127-136, 150-156, 158-166, 223-233, 245-263, 291-296, 9-126 and 127-285 of SEQ ID NO:190; 30-43, 46-56, 61-70, 72-83, 85-93, 103-113, 119-125, 151-166, 179-191, 212-218, 225-231, 236-243, 262-267, 291-307, 331-344, 349-355, 366-372, 380-386, 414-422, 428-447, 459-464, 469-478, 507-519, 525-544, 563-569, 576-590, 620-626, 633-643, 654-659, 665-671, 684-707, 717-723, 725-733, 747-779, 782-801 and 347-361 of SEQ ID NO:191; 4-12, 14-26, 37-80, 107-115, 133-139, 144-150, 154-165, 173-180, 191-199, 205-211, 221-231, 237-244, 254-284, 307-340, 342-353, 360-368, 370-380, 479-493, 495-503, 509-522, 525-536, 539-547, 554-560, 565-573, 578-583, 7-23 and 465-479 of SEQ ID NO:192; 4-17, 47-55, 76-83, 85-100, 104-112, 117-123, 126-135, 142-148, 156-167, 174-182, 267-273 and 258-283 of SEQ ID NO:193; 8-32, 36-42, 65-88, 102-108, 112-140, 147-163, 170-179, 183-193 and 117-124 of SEQ ID NO:194; 12-18, 45-50, 62-77, 82-95, 99-113, 115-123, 125-147, 155-177, 187-209, 211-223, 244-253, 259-270, 278-297, 302-307, 311-318, 329-334, 350-356, 359-365, 390-400, 402-413 and 333-350 of SEQ ID NO:195; 4-13, 15-27, 30-46, 53-58, 68-74, 82-95, 115-126, 134-139, 148-153, 159-176, 182-199, 201-217, 220-225, 227-235, 237-248, 253-266, 300-315, 322-336, 390-396, 412-426, 438-445, 448-459, 477-484, 502-508, 515-527, 529-537, 553-568, 643-651, 658-667, 690-703 and 376-400 of SEQ ID NO:196; 4-10, 24-32, 38-55, 59-67, 70-77, 80-87, 89-97, 123-129, 134-151, 166-172, 178-189, 191-216, 218-235, 245-259, 271-315, 326-339, 341-360 and 73-94 of SEQ ID NO:197; 13-25, 31-38, 43-57, 79-85, 92-99, 106-112, 117-128, 130-139, 146-158, 160-175, 194-204, 211-222, 225-232, 234-242, 263-270, 278-292, 299-320, 322-333 and 240-256 of SEQ ID NO:198; 4-17, 55-63, 66-101, 109-131, 135-143, 145-151, 155-161, 164-170, 177-185, 192-198, 213-218, 223-238, 246-256, 258-268, 273-283, 309-314, 322-328 and 195-221 of SEQ ID NO:199; 13-24, 31-39, 41-50, 63-69, 90-96, 104-109, 116-141, 148-153, 161-167, 173-178, 190-209, 253-258, 265-272, 279-289, 295-312, 317-343, 355-366, 376-389, 400-407, 430-451, 453-464, 466-472, 487-493, 499-505, 523-538, 554-559, 568-579, 584-601 and 344-363 of SEQ ID NO:200; 5-22, 30-36, 53-59, 61-70, 82-92, 99-106, 120-131, 135-148, 154-167, 169-183, 187-199, 204-212, 231-247 and 111-249 of SEQ ID NO:201; 17-36, 40-66, 71-144, 148-171, 173-191, 199-214, 220-252, 265-272, 278-288, 298-333, 342-385 and 287-307 of SEQ ID NO:202; 4-16, 22-28, 30-36, 42-48, 95-116, 154-162, 164-174, 239-252, 258-263, 273-285, 306-313, 323-333, 341-357, 363-369, 372-379, 395-401, 430-436, 438-453, 464-480, 33-44, 233-258 and 349-369 of SEQ ID NO:203; 4-21, 30-37, 46-53, 59-68, 80-92, 98-104, 118-143, 150-160, 165-185, 187-200, 204-211, 224-236, 241-246, 252-258, 271-280, 288-294, 311-320, 335-341 and 191-350 of SEQ ID NO:204; 4-16, 37-59, 64-70, 79-87, 93-102, 107-127, 143-165, 172-188, 197-204, 207-218, 221-227, 242-248, 258-277, 289-296, 298-316, 332-338, 344-365, 367-373, 375-382, 400-408, 415-425, 438-446 and 235-250 of SEQ ID NO:205; 4-37, 39-66, 84-98, 101-127, 140-149, 157-163, 166-172, 175-182, 184-193, 203-208, 215-232, 234-247, 250-299, 303-345 and 183-204 of SEQ ID NO:206; 10-20, 41-61, 73-87, 112-141, 176-192, 194-201, 205-222, 230-237, 257-264, 276-282, 284-310, 312-318, 330-337, 349-357 and 304-328 of SEQ ID NO:207; 4-31, 42-103, 105-113, 121-153, 160-181, 188-196, 210-226, 231-264, 272-287, 297-304, 328-336 and 304-318 of SEQ ID NO:208; 21-43, 46-52, 54-70, 72-79, 94-107, 133-141, 160-166, 217-253, 311-317, 359-365, 374-381, 390-395, 434-440, 488-494, 497-502, 511-522, 554-563, 565-574, 577-585, 591-598, 601-606, 617-625, 633-643, 658-664, 676-682, 694-702, 710-719, 754-760, 782-788, 802-808, 916-921, 942-948, 955-964, 973-979, 992-998, 1006-1011, 1016-1023, 1030-1038, 1046-1053, 1059-1066, 1088-1098, 1119-1126, 1129-1135, 1156-1171, 1173-1181, 1202-1210, 1255-1261, 1268-1280, 1295-1310, 1312-1320, 1375-1381, 1406-1417, 1450-1471, 1478-1492, 1498-1506, 1569-1578, 1603-1608, 1611-1624, 1648-1655, 1663-1670, 1680-1698, 1702-1707, 1713-1719, 1737-1742, 1747-1753, 1762-1769, 1771-1785, 1790-1804, 1811-1818, 1830-1836, 1838-1852, 1874-1886, 1893-1899, 1902-1909, 1942-1948, 1952-1962, 1980-1986, 2001-2017, 2020-2028, 2042-2050, 2052-2068, 2074-2079, 2083-2095, 2107-2113, 2147-2155, 2177-2194, 2203-2211, 2236-2241, 2251-2258, 2267-2274, 2285-2292, 2314-2328, 2330-2340, 2358-2365, 2390-2401, 2408-2418, 2432-2453, 2463-2476, 2486-2507, 2528-2537, 2540-2548, 2552-2558, 2568-2576, 2596-2601, 2610-2622, 2629-2638, 2653-2669, 2718-2727, 2749-2767, 2777-2784, 2789-2795, 2806-2815, 2817-2824, 2835-2843, 2847-2854, 2860-2881, 511-523, 612-630 and 1790-1803 of SEQ ID NO:209; 4-54, 61-68, 72-82, 86-93, 100-108, 115-130, 147-154, 187-194, 196-207, 224-229, 236-251, 275-287 and 96-109 of SEQ ID NO:210; 31-39, 62-69, 91-101, 158-172, 175-180, 186-193, 201-208, 210-223, 243-250, 273-286, 293-299, 319-325, 343-354, 356-365, 368-384, 414-435, 471-491, 512-518, 550-556, 567-581, 584-589, 633-639, 680-692, 697-708, 716-721, 747-754, 779-786, 810-816 and 366-503 of SEQ ID NO:211; 5-20, 22-48, 57-65, 96-101, 111-122, 130-145, 154-164, 170-181, 193-199, 201-216, 224-241, 244-262, 281-323, 342-351, 359-367, 369-396, 406-416, 424-433, 450-456, 485-491, 493-499, 501-515, 517-535 and 289-305 of SEQ ID NO:212; 4-17, 22-44, 53-60, 66-83, 87-94, 101-106, 110-116, 131-137, 148-183, 189-207, 209-215, 233-242, 251-262, 264-272, 290-296, 308-327, 359-373, 375-380, 397-405, 415-420, 426-433, 444-475, 478-484, 529-536, 548-558 and 106-126 of SEQ ID NO:213; 4-38, 42-50, 58-64, 72-81, 92-118, 140-146, 157-165, 172-192, 198-204, 208-216, 227-234, 238-258, 271-278, 288-293, 311-322, 327-346, 357-370, 375-383, 395-409, 411-417, 425-432, 436-445, 109-129 and 370-380 of SEQ ID NO:214; 23-30, 36-49, 52-64, 86-94, 97-104, 121-129, 257-272, 279-286, 288-294, 307-327, 334-340, 369-375, 377-386, 406-412, 418-423, 430-438, 441-447, 459-465, 469-476, 482-488, 510-546, 550-580, 584-622, 638-645, 653-659, 675-683, 692-705, 723-731, 752-761, 788-795 and 54-72 of SEQ ID NO:215; 11-33, 36-46, 88-104, 116-126, 134-170, 189-195, 199-217, 225-250, 255-261, 266-273, 280-291, 296-313, 334-341, 343-349, 354-360, 362-369, 373-380, 387-401, 406-420 and 259-273 of SEQ ID NO:216; 9-14, 28-44, 57-64, 72-79, 86-93, 104-111, 116-126, 142-150, 159-164 and 61-86 of SEQ ID NO:217; 10-17, 26-33, 43-61, 69-95, 101-107, 109-125, 129-135, 137-144, 147-153, 158-169, 177-187, 209-219, 221-232, 235-247, 261-268, 271-282, 296-302, 306-347, 355-362, 364-379, 386-399, 409-418, 424-442, 451-460, 467-479, 490-498 and 60-74 of SEQ ID NO:218; 8-14, 20-31, 65-84, 94-99, 154-179, 193-207, 238-253 and 96-118 of SEQ ID NO:219; 4-24, 30-44, 47-62, 84-93, 108-116, 124-133, 136-141, 201-209, 217-223, 228-235, 238-245, 247-270, 275-285, 290-314, 328-338, 342-349, 353-365, 375-383, 386-392, 394-402, 417-427, 443-459, 465-481, 492-514, 516-524, 550-566, 602-617, 630-639, 666-676, 687-693, 719-730, 747-753, 783-790, 799-816, 824-831, 837-842 and 167-189 of SEQ ID NO:220; 6-15, 18-28, 58-66, 84-101, 106-129, 136-151, 154-165, 182-203, 205-211, 214-220, 222-228, 233-240, 251-260, 270-277, 284-291, 306-315, 322-328, 363-369, 378-388, 392-405, 443-452, 495-501, 512-523, 574-583 and 362-375 of SEQ ID NO:221; 5-25, 27-34, 47-59, 64-70, 76-86, 145-158, 166-183, 189-202, 217-231, 235-242, 260-270, 278-309 and 1-102 of SEQ ID NO:222; 4-19, 24-76, 78-83, 90-99, 102-109, 114-122, 137-147, 154-174, 177-188, 203-212, 217-223, 227-239 and 226-325 of SEQ ID NO:223; 7-37, 71-90, 94-109, 117-128, 141-153, 179-192, 199-206, 225-231, 237-243, 258-264 and 40-51 of SEQ ID NO:224; 13-19, 25-30, 46-59, 75-91, 101-107, 114-124, 129-135, 137-145, 160-167, 171-179, 187-194, 209-215, 217-222, 229-239, 243-249, 257-265, 269-275, 299-308, 310-327 and 282-300 of SEQ ID NO:225; 86-100, 216-230, 342-369, 382-388, 424-430, 438-445, 452-458, 488-494, 501-518, 554-560, 568-574, 584-592, 603-609, 611-629, 639-645, 652-661, 669-699, 708-714, 726-738, 747-753, 763-775, 785-791, 794-807, 815-824, 826-845, 854-860, 863-868, 870-883, 892-898, 901-906, 909-921, 930-937, 946-959, 968-974, 977-990, 998-1007, 1009-1027, 1037-1043, 1046-1051, 1053-1066, 1075-1081, 1084-1089, 1092-1103, 1113-1119, 1122-1135, 1143-1152, 1154-1172, 1182-1188, 1191-1196, 1200-1210, 1220-1226, 1229-1235, 1237-1249, 1259-1265, 1268-1281, 1289-1298, 1305-1318, 1328-1334, 1337-1343, 1345-1357, 1367-1373, 1390-1396, 1405-1411, 1418-1423, 1426-1435, 1445-1455, 1474-1483, 1493-1500, 1505-1512, 1517-1524, 1538-1544, 1568-1578, 1595-1601, 1674-1682, 1687-1720, 1728-1736, 1738-1744, 1754-1761, 1764-1774, 1798-1824, 1836-1842, 1886-1893, 1895-1903, 366-781, 782-1518 and 1731-1747 of SEQ ID NO:226; 4-17, 20-39, 46-55, 60-66, 102-110, 114-122, 125-131, 161-167, 172-178, 185-190, 195-202, 218-232, 236-252, 264-291, 293-302, 309-315, 324-339 and 169-381 of SEQ ID NO:227; 5-10, 13-40, 42-53, 69-75, 83-89, 120-135, 150-161, 174-190, 203-225, 229-247, 257-287, 318-348 and 30-200 of SEQ ID NO:228; 7-19, 43-53, 64-72, 124-139, 52-84 and 120-131 of SEQ ID NO:229; 12-19, 39-48, 58-100, 117-123, 154-162, 164-187, 189-195, 202-216, 218-235, 241-246, 262-278, 315-328, 333-347, 354-366, 372-379, 391-405, 422-429, 431-442, 444-450, 458-466, 478-485, 494-501, 504-510, 520-535, 573-580, 589-598, 615-625, 666-676, 686-698, 722-729, 737-746, 756-767, 787-796, 805-816, 824-829, 833-848, 856-864, 866-876, 879-886, 898-904, 918-924, 927-934, 941-960, 967-978 and 561-575 of SEQ ID NO:230; 11-29, 49-55, 70-77, 84-100, 102-112, 148-155, 160-177, 181-204 and 1-104 of SEQ ID NO:231; 27-44, 64-71, 122-133, 151-156, 164-178, 214-220, 226-232, 235-244, 253-262, 282-288, 294-310, 317-325, 350-356, 362-368, 376-383, 438-443, 449-454, 459-464, 492-498, 500-511, 529-535, 538-546, 567-573, 597-603, 660-665, 674-679, 724-734, 763-769, 773-784, 791-801, 807-815, 821-826, 840-848, 863-868, 897-902, 908-928, 932-953, 956-975, 980-987, 990-996, 1012-1018, 1042-1063, 1095-1116, 1149-1157, 1160-1167, 110-357, 358-501 and 502-1161 of SEQ ID NO:232; 4-21, 64-71, 73-84, 128-138, 144-162, 203-217, 240-263, 288-298, 300-308, 310-317, 325-351, 369-380, 391-411 and 330-345 of SEQ ID NO:233; 5-11, 25-31, 39-48, 51-79, 89-98, 100-122, 135-148, 166-201, 203-227, 230-250, 254-260, 266-272, 274-282, 299-305, 328-337 and 31-45 of SEQ ID NO:234; 12-23, 29-48, 51-60, 66-72, 75-81, 83-93, 103-115, 133-148, 168-174, 195-204, 222-229, 231-240, 242-251, 270-280, 286-305, 322-344, 349-360, 364-370, 378-400, 421-441, 448-484, 486-493, 495-501, 504-534, 547-561, 567-590, 597-607, 621-635, 643-649, 658-685, 688-694, 702-711, 717-731, 737-742, 759-765, 767-772, 776-786, 803-809, 815-825, 854-908, 910-919, 923-930, 942-948, 961-975, 994-1014 and 915-940 of SEQ ID NO:235; 4-9, 32-47, 51-61, 75-96, 139-191 and 1-124 of SEQ ID NO:236; 4-13, 17-38, 43-49, 55-76, 88-95, 110-121, 128-146, 151-157, 162-214, 222-240, 243-249, 251-273, 275-281, 292-298, 300-309, 312-320, 322-331, 355-369, 376-408, 446-460, 471-482, 485-509 and 191-203 of SEQ ID NO:237; 4-21, 72-82, 89-103, 106-115, 118-124, 140-146, 174-184, 191-200, 204-213, 218-224, 261-266, 282-293, 299-309, 311-340, 342-358, 362-372, 381-389, 391-402, 413-421, 438-447, 457-464, 470-478, 501-507, 545-560, 578-624, 631-641, 658-670, 680-689, 717-738, 753-759, 795-805, 816-822, 830-838, 842-848, 869-881, 892-898, 33-51 and 818-835 of SEQ ID NO:238; 4-21, 79-85, 156-177, 183-188, 206-214, 243-249, 261-269, 287-292, 315-322, 334-345, 360-366, 374-390, 402-411, 37-97 and 260-399 of SEQ ID NO:239; 4-9, 19-54, 58-78, 97-104, 111-120, 126-134, 137-145, 163-173, 178-188, 193-203, 211-224, 246-286, 288-324, 337-346, 355-362, 374-390, 392-398, 409-417 and 240-249 of SEQ ID NO:240; 5-12, 14-31, 35-41, 43-61, 82-92, 97-105, 134-145, 155-166, 184-203, 215-223, 225-251, 272-279, 281-306, 310-345, 358-418, 435-473, 482-490, 525-532, 538-547, 549-563, 578-604, 613-639 and 144-154 of SEQ ID NO:241; 53-59, 64-72, 74-100, 133-152, 154-172, 176-181, 207-214, 225-238, 275-297, 304-310, 331-340, 362-367, 384-395, 403-410, 437-443, 448-456, 482-490, 579-597, 602-610, 625-630, 633-651, 699-707, 709-715, 734-743, 750-762 and 544-685 of SEQ ID NO:242; 12-18, 22-40, 45-83, 89-97, 103-109, 147-153, 159-173, 195-204, 210-219, 243-253, 259-265, 273-282, 303-309, 315-325, 332-340, 346-358, 362-367, 377-390, 393-402, 418-426, 447-455, 467-480, 505-512, 514-525, 548-561, 566-576, 584-596, 619-626, 638-645, 649-659, 661-680, 699-708, 714-720, 753-759, 766-772, 775-781, 801-808, 202-218, 282-299, 339-350 and 617-628 of SEQ ID NO:243; 5-33, 52-62, 87-101, 111-135, 137-143, 145-152, 190-202, 209-221, 233-245, 253-270 and 151-215 of SEQ ID NO:244; 19-29, 32-39, 42-48, 75-94, 124-135, 137-145, 152-160, 176-182, 193-203, 215-236, 266-273, 275-291, 297-306, 311-319, 322-342, 348-360, 369-378, 394-401 and 48-64 of SEQ ID NO:245; 4-11, 13-33, 36-43, 53-63, 65-80, 112-129, 134-141, 143-155, 157-168, 178-188, 191-199, 201-207, 215-229, 242-255, 263-270, 283-315, 320-329, 333-338, 340-349, 412-426, 465-478, 485-490, 498-512, 540-554 and 390-516 of SEQ ID NO:246; 4-18, 23-32, 41-47, 54-70, 88-99, 104-111, 118-138, 143-148, 150-162, 168-175, 181-188, 203-211, 214-220, 227-245, 251-268, 275-281, 287-296, 323-333 and 1-90 of SEQ ID NO:247; 8-34, 38-49, 72-83, 85-91, 94-104, 112-125, 134-142, 148-168, 181-189, 191-198, 202-214, 222-233, 242-254, 256-262, 273-278, 287-294, 314-325 and 141-159 of SEQ ID NO:248; 4-24, 30-36, 47-75, 82-105, 124-134, 151-157, 192-202, 208-214, 219-226, 234-247, 285-290, 318-324, 332-340, 343-349, 380-386, 453-462, 472-478, 484-501, 531-540, 550-557, 604-612, 620-625, 642-648, 652-671, 64-84, 93-180 and 181-446 of SEQ ID NO:249; 12-18, 24-32, 68-75, 77-83, 96-101, 109-116, 129-136, 152-164, 175-184, 190-199, 206-215, 224-233, 241-250, 258-264, 273-292, 302-312, 319-331, 334-346, 348-368, 387-395, 408-416, 420-429, 437-452 and 364-374 of SEQ if D NO:250; 11-28, 36-52, 60-67, 74-79, 108-116 and 61-76 of SEQ ID NO:251; 20-27, 38-49, 69-74, 84-107, 138-145, 161-168, 179-195, 210-226, 228-252, 267-281, 283-296, 305-311, 333-340, 342-356, 361-372, 380-399, 401-414, 458-466, 475-481, 492-507, 515-520 and 146-160 of SEQ ID NO:252; 43-61, 68-74, 76-90, 120-128, 130-149, 156-161, 164-182, 206-234, 242-252, 269-274, 291-304, 332-345, 349-355, 360-371, 374-388, 434-440, 447-453, 459-465, 469-496, 504-522 and 261-285 of SEQ ID NO:253; 4-17, 24-30, 37-49, 87-98, 118-124, 126-136, 144-171, 176-188, 206-214, 216-228, 233-240, 246-252, 262-271, 277-297, 307-330, 333-342, 346-352, 355-361, 368-386, 391-400, 413-420, 474-480 and 401-427 of SEQ ID NO:254; 15-26, 31-46, 51-72, 80-93, 96-109, 131-137, 150-158, 179-185, 189-209, 211-219, 221-234, 241-247, 255-262, 265-271, 283-288 and 173-190 of SEQ ID NO:255; 28-37, 39-45, 51-58, 77-84, 89-97, 132-148, 171-180, 199-205, 212-218, 220-226, 257-265, 273-300, 307-327, 334-340, 344-365, 385-390, 402-408, 426-436, 450-468, 476-485 and 425-497 of SEQ ID NO:256; 4-25, 70-76, 80-88, 90-100, 120-128, 162-169, 183-203, 261-277, 279-289, 291-297, 302-308, 321-327, 339-353, 358-377, 392-401, 404-410, 414-422, 443-450, 456-461, 470-488, 490-497, 510-535, 570-611, 618-630, 639-647, 649-660, 668-690, 702-716, 718-724, 737-747, 750-764 and 497-509 of SEQ ID NO:257; 12-48, 50-64, 99-108, 216-223, 235-241, 244-254, 262-274, 287-293, 310-316, 320-326, 361-366, 377-383, 390-395, 408-414, 418-425, 438-444, 462-469, 494-505, 524-530, 536-547, 551-566, 592-598, 601-613, 678-685, 687-695, 709-717, 727-737, 751-757, 760-765, 772-778, 782-788, 801-807, 822-830, 859-868, 870-878, 884-890, 898-903, 909-919, 953-969, 973-980, 990-1000, 1002-1019, 1041-1047, 1059-1065, 1090-1095, 1116-1127, 1130-1139, 1143-1149, 1151-1168, 1178-1183, 1188-1195, 1197-1209, 1213-1220, 1226-1234, 1236-1247, 1255-1274, 1276-1282, 76-100, 270-284, 309-438, 493-505, 786-942 and 947-967 of SEQ ID NO:258; 4-9, 24-34, 46-95, 97-109, 119-130 and 138-156 of SEQ ID NO:259; 9-26, 28-35, 43-53, 55-68, 83-92, 99-105, 110-135, 139-149, 157-162, 164-170, 173-183, 193-208, 210-230, 239-245, 253-259, 263-271, 293-305, 310-320, 322-331, 336-343, 351-364, 367-376, 92-107 and 154-173 of SEQ ID NO:260; 19-39, 52-62, 108-117, 145-152, 160-168, 194-203, 229-240, 252-268, 280-287, 308-316, 333-339, 383-390, 403-412, 414-424, 438-445, 464-472, 479-484, 489-505, 510-526 and 247-260 of SEQ ID NO:261; 5-17, 25-52, 60-77, 105-113, 118-125, 162-167, 228-234, 272-279, 328-334, 341-357, 381-395, 400-406, 512-518, 557-569, 586-592, 645-651, 690-695, 701-709, 720-726, 733-743, 751-758, 781-786, 879-886, 929-934, 939-944, 952-960, 965-975, 994-1001, 1039-1045, 1102-1109, 1164-1181, 1198-1206, 1223-1229, 1253-1259, 1283-1292, 1312-1317, 1339-1349, 1360-1370, 1389-1398, 1400-1412, 1452-1465, 1470-1484, 1490-1497, 1519-1525, 1554-1564, 1578-1591, 1623-1636, 1638-1646, 1669-1679, 1685-1697, 1704-1711, 1713-1720, 1730-1736, 1738-1749, 1756-1764, 1778-1786, 1796-1803, 1817-1826, 1849-1866, 1975-1993, 2017-2032, 2044-2053, 2070-2086, 2091-2109, 2116-2127, 2156-2167, 2182-2188, 2197-2202, 2244-2252, 2281-2287, 2290-2307, 2350-2361, 2383-2404, 2425-2433, 2445-2455, 2495-2505 and 394-549 of SEQ ID NO:262; 9-24, 31-53, 57-67, 69-79, 84-114, 133-141, 144-172, 178-186 and 13-46 of SEQ ID NO:263; 4-25, 27-35, 43-52, 59-70, 79-91, 115-130, 136-152, 154-163, 170-179 and 1-58 of SEQ ID NO:264; 4-30, 49-55, 71-80, 96-105, 111-126, 139-146, 149-162, 239-245, 279-285, 290-296, 300-307, 331-337, 343-350 and 250-351 of SEQ ID NO:265; 9-27, 34-41, 43-51, 92-111, 114-120, 123-131, 139-150, 156-171, 176-186, 188-204, 229-241, 252-258, 266-279, 288-297, 319-334, 338-348, 373-379, 389-398, 431-439, 479-484 and 214-398 of SEQ ID NO:266; 4-15, 18-27, 47-52, 68-83, 91-97, 104-110, 115-121, 139-147, 157-164, 198-206, 227-236, 241-254, 264-273, 278-289, 311-320, 353-361, 372-383, 405-420, 426-434 and 232-386 of SEQ ID NO:267; 4-10, 24-34, 91-97, 129-141, 156-163, 184-190, 205-219, 229-235, 256-273, 278-285 and 93-116 of SEQ ID NO:268; 7-29, 35-54, 71-83, 85-91, 104-111, 122-134, 138-144, 146-154, 158-174, 177-183, 186-201, 207-215, 223-235, 240-247, 262-273, 275-283, 287-292 and 48-66 of SEQ ID NO:269; 7-27, 31-47, 49-70, 75-102, 110-149, 157-171, 217-223, 235-251, 294-302, 358-364, 367-375, 387-393, 395-412, 423-430, 441-451, 456-470, 472-486, 488-495, 499-509, 515-529, 536-549, 556-570, 574-603, 607-615, 625-633, 642-658, 670-676, 683-702, 708-716, 720-726, 747-756, 763-784, 803-812, 815-826 and 475-490 of SEQ ID NO:270; 7-22, 30-38, 53-59, 64-75, 83-95, 97-112, 120-131, 133-142, 145-151, 154-166, 172-180, 189-203, 227-238, 277-287, 9-156 and 174-287 of SEQ ID NO:271; 13-23, 25-32, 111-117, 150-164, 185-193, 207-212, 216-224, 230-236, 263-272, 304-311, 342-348, 374-385, 391-407, 444-458, 480-487, 489-499, 523-542, 544-558, 572-579, 620-640, 686-696, 703-710, 742-755, 765-772, 817-822, 830-837, 865-872, 931-937 and 66-86 of SEQ ID NO:272; 4-27, 49-56, 62-70, 86-92, 121-127, 151-163, 170-182, 195-202, 212-226, 237-243 and 234-254 of SEQ ID NO:273; 4-10, 13-24, 39-51, 62-78, 92-104, 107-117, 134-141, 156-161, 166-181, 210-216, 222-229, 256-266, 273-280, 297-304, 313-330, 336-349, 371-376, 433-439, 443-448, 488-493, 506-515, 527-534, 560-572, 575-583, 587-593 and 252-483 of SEQ ID NO:274; 4-15, 21-38, 45-56, 81-95, 102-108, 118-130, 133-147, 152-162, 166-171, 199-204, 211-218, 230-240, 253-261, 274-283, 288-294, 312-317, 325-336, 344-357, 391-414 and 24-146 of SEQ ID NO:275; 26-31, 38-56, 65-82, 90-101, 112-119, 123-153, 175-188, 197-216, 234-242, 249-265, 273-286, 290-305, 327-335, 338-346, 361-372, 394-404 and 290-306 of SEQ ID NO:276; 17-26, 43-48, 50-73, 81-93, 95-107, 139-146, 158-168, 171-176, 190-196, 202-212, 216-223, 243-266, 274-282, 308-313, 324-330, 344-378, 380-387, 403-422, 427-443, 448-455, 457-465, 491-515, 517-528, 553-567, 589-599, 610-617, 642-648, 670-697, 709-717, 726-743, 745-759, 769-803, 807-823, 840-849 and 820-851 of SEQ ID NO:277; 4-18, 39-48, 53-63, 66-90, 102-117, 125-134, 137-145, 156-162, 169-197, 26-40 and 56-80 of SEQ ID NO:278; 21-33, 36-42, 49-60, 68-76, 91-105, 123-130, 141-161, 169-178, 185-190, 192-199, 205-214, 223-233, 239-247, 260-269, 284-293, 300-314, 324-352, 357-364, 373-382, 389-403, 420-432, 438-446, 466-471, 477-484, 503-509, 549-556, 558-576, 600-623, 625-635, 654-661, 663-669, 671-687, 702-716, 735-741, 744-750, 757-766, 776-786, 807-815, 824-832, 854-860, 863-897, 909-915, 920-946, 952-959, 982-997, 1024-1038, 1049-1055, 1071-1085, 1104-1113, 1121-1132, 1138-1150, 1187-1196, 1212-1221, 1227-1236, 1257-1262, 1264-1278, 1282-1294, 1307-1318, 1353-1370, 1382-1388, 1396-1409, 1434-1440, 1446-1454, 1465-1478, 1485-1513, 1516-1529, 1540-1545, 1563-1568, 1575-1593, 1607-1616, 1628-1645, 1648-1661, 1676-1682, 1689-1697, 1713-1719, 1739-1749, 1753-1758, 1763-1774, 1797-1803, 1807-1846, 1855-1874, 1877-1891, 1893-1907, 1912-1925, 1931-1943, 1955-1965, 1976-1990, 2032-2043, 2045-2051, 2099-2105, 2131-2138, 2161-2179, 2188-2199, 2205-2216, 2219-2227, 2235-2245, 2247-2267, 2277-2288, 2294-2304, 2314-2326, 2346-2358, 2365-2377, 2383-2402, 2407-2423, 2437-2450, 2454-2473, 2489-2497, 2525-2531, 2557-2570, 2580-2587, 2589-2599, 2621-2641, 2647-2653, 2661-2677, 2685-2690, 2697-2717, 2722-2733, 2739-2777, 2786-2793, 2801-2808, 2811-2822, 2825-2835, 2838-2845, 2859-2871, 2877-2883, 213-344, 954-1080 and 2524-2733 of SEQ ID NO:279; 10-16, 18-23, 28-41, 63-69, 77-91, 101-109, 118-136, 146-153, 155-162, 168-179, 192-207, 217-226, 229-235, 239-254, 279-286, 294-307, 313-319, 334-341, 344-353, 363-377, 390-396 and 178-328 of SEQ ID NO:280; 18-42, 68-84, 89-95, 100-105, 107-115, 125-135, 154-177, 189-195, 205-228, 236-243, 252-259, 279-300, 309-316, 323-331, 340-351, 353-364, 377-402 and 85-97 of SEQ ID NO:281; 4-18, 26-32, 66-76, 100-126, 151-159, 178-186, 188-194, 200-210, 241-248, 253-259, 262-279, 284-291, 307-313, 315-322, 327-337, 376-386, 399-407, 432-441, 467-473, 487-497, 499-505, 543-549, 560-568, 585-593, 598-604, 608-614, 630-642, 647-653, 690-703, 717-730, 21-200 and 468-480 of SEQ ID NO:282; 17-49, 52-58, 62-73, 78-97, 100-117, 122-172, 185-190, 193-217, 225-236 and 33-42 of SEQ ID NO:283; 7-39, 50-58, 73-89, 96-107, 109-120, 126-142, 152-170, 178-202, 205-211, 224-244, 249-259, 261-270, 300-310, 312-325 and 158-169 of SEQ ID NO:284; 4-31, 40-64, 71-82, 85-92, 102-124, 126-139, 147-152, 159-173, 176-188, 195-207, 210-216, 234-241, 249-256, 258-276, 279-293, 296-302, 310-315, 349-356, 363-378, 380-403, 411-426, 435-441, 448-459, 463-476, 488-494 and 201-221 of SEQ ID NO:285; 5-13, 15-74, 87-104, 107-120, 123-129, 136-145, 150-191, 193-206, 227-248, 250-264, 278-302, 304-323, 332-378, 384-407, 409-419, 425-457, 462-471, 474-497, 511-545, 555-564, 571-578, 585-598, 640-647, 669-675, 682-691, 693-705, 729-743, 752-761, 772-780, 786-804, 808-818, 822-846, 858-880, 884-900, 910-939, 941-947, 962-971, 973-988, 998-1003, 1007-1027 and 236-259 of SEQ ID NO:286; 4-19, 27-68, 81-111, 121-160 and 60-79 of SEQ ID NO:287; 4-37, 40-46, 52-57, 199-205, 222-229, 236-244, 250-267, 269-282 and 27-197 of SEQ ID NO:288; 4-16, 24-30, 32-38, 63-75, 86-92, 98-111, 113-126, 160-165, 170-180, 198-204, 227-233, 239-245, 253-273, 308-314, 352-365, 382-387, 395-403, 423-429, 472-482, 484-493, 501-507, 518-526, 536-541, 543-550, 556-562, 586-600, 626-633, 649-661, 680-688 and 546-559 of SEQ ID NO:289; 16-33, 48-59, 63-71, 77-92, 94-109, 117-124, 139-151, 169-181, 184-227, 233-249, 251-261, 263-275, 282-294, 297-321, 326-332, 341-355, 383-399 and 258-272 of SEQ ID NO:290; 11-26, 31-39, 43-52, 55-62, 64-70, 80-94, 123-133, 135-141, 172-181, 185-206, 209-218, 224-230, 238-244, 251-262, 264-271, 290-301, 306-324, 333-340, 350-357, 367-375, 390-397, 434-441, 443-448, 77-226 and 350-429 of SEQ ID NO:291; 4-13, 22-27, 31-45, 50-59, 72-96, 99-114, 131-141, 143-150, 159-176, 180-186, 189-198, 208-214, 234-253, 271-287, 294-299, 310-366, 382-390, 398-416, 424-443 and 283-305 of SEQ ID NO:292; 9-26, 30-53, 62-72, 86-95, 112-122, 136-145, 153-160, 209-221, 227-237, 241-268, 281-288, 291-298, 308-314, 321-328, 336-346, 351-379, 388-397, 409-416, 423-433, 443-481, 511-519 and 213-232 of SEQ ID NO:293; 12-18, 25-31, 38-50, 59-67, 71-82, 96-126 and 76-88 of SEQ ED NO:294; 4-25, 39-44, 64-71, 74-88, 100-113, 128-138, 151-162, 164-177, 185-190, 204-213, 233-239, 246-254, 281-286, 293-306, 309-318, 333-347, 349-359, 385-398, 404-423, 458-465, 477-484, 490-499, 501-533, 554-566, 582-590, 596-616, 624-629, 631-639, 654-680, 694-720, 735-743 and 2-100 of SEQ ID NO:295; 4-16, 36-41, 52-75, 98-107, 109-117, 122-128, 133-139, 141-155, 159-165, 169-182, 187-193, 195-201, 211-224, 230-236, 247-269, 278-290 and 75-92 of SEQ ID NO:296; 7-21, 25-33, 37-43, 87-94, 103-120, 131-147, 168-174, 197-203, 207-212, 227-237, 247-257, 263-271, 279-287, 298-306, 320-325, 332-340, 363-374, 379-384, 390-401, 403-414, 428-433, 448-457, 462-475, 483-490, 513-519, 525-535, 543-554, 559-566, 571-620, 625-631, 636-642, 659-670, 688-706, 708-723, 770-779, 787-793, 796-807, 820-840, 848-854, 863-874, 895-905, 912-919, 934-942, 968-975, 983-1000, 1012-1019, 1026-1036, 1050-1060, 1064-1070, 1081-1091, 1094-1108, 1112-1118, 1140-1152, 1164-1169, 1172-1180, 1187-1192 and 732-748 of SEQ ID NO:297; 23-40, 42-59, 66-73, 78-97, 111-128, 130-141, 157-166, 178-183 and 53-71 of SEQ ID NO:298; 4-27, 38-44, 47-57, 59-85, 99-106, 114-121, 154-166, 181-186, 193-198, 238-244, 253-262, 272-278, 287-299, 314-320, 338-350, 358-368, 382-388, 407-416, 433-446, 456-461, 463-473 and 86-195 of SEQ ID NO:299; 5-24, 38-59, 64-80, 87-99, 105-126, 134-142, 149-163, 165-179, 181-202, 205-220, 227-233, 243-250, 257-263 and 87-245 of SEQ ID NO:300; 5-32, 47-53, 66-79, 81-97, 115-151, 155-174, 183-188, 196-210, 215-226, 230-238, 253-258, 263-270, 276-282, 295-301, 304-325, 334-344, 360-390, 397-412, 425-432, 434-462, 478-494, 508-526, 539-564, 571-579, 347-371 and 375-386 of SEQ ID NO:301; 4-15, 36-44, 49-56, 60-66, 68-82, 84-103, 109-115, 118-141, 147-154, 160-168, 176-185 and 26-39 of SEQ ID NO:302; 7-13, 23-33 and 13-21 of SEQ ID NO:303; 2-10 of SEQ ID NO:304; 4-9, 12-18, 35-42, 49-62 and 6-18 of SEQ ID NO:305; 19-25 and 1-13 of SEQ ID NO:306; 15-21, 27-45 and 12-25 of SEQ ID NO:307; 14-20 and 1-14 of SEQ ID NO:308; 4-18 and 13-26 of SEQ ID NO:309; 8-21 and 2-20 of SEQ ID NO:310; 4-14 and 4-16 of SEQ ID NO:311; 3-12 of SEQ ID NO:312; 6-14, 6-25, 35-57 and 2-14 of SEQ ID NO:313; 6-25, 35-57 and 17-31 of SEQ ID NO:314; 14-25, 32-46 and 5-19 of SEQ ID NO:315; 18-31 and 5-16 of SEQ ID NO:316; 19-24 and 4-26 of SEQ ID NO:317; 13-21, 29-34, 47-58, 61-73 and 36-47 of SEQ ID NO:318; 4-15 and 5-24 of SEQ ID NO:319; 6-18 of SEQ ID NO:320; 13-20 and 4-13 of SEQ ID NO:321; 15-23 of SEQ ID NO:322; 4-9 and 7-21 of SEQ ID NO:323; 1-10 of SEQ ID NO:324; 4-14 of SEQ ID NO:325; 4-17, 35-41, 46-89, 93-98 and 70-88 of SEQ ID NO:326; 1-13 of SEQ ID NO:327; 4-16, 26-32 and 25-38 of SEQ ID NO:328; 8-15, 23-28 and 4-17 of SEQ ID NO:329; 4-12 and 1-15 of SEQ ID NO:330; 4-29, 31-42, 52-58 and 6-16 of SEQ ID NO:331; 4-9, 24-32 and 9-19 of SEQ ID NO:332; 4-12, 18-27 and 5-18 of SEQ ID NO:333; 4-11, 37-56, 58-92 and 18-29 of SEQ ID NO:334; 8-28 and 20-35 of SEQ ID NO:335; 4-15 of SEQ ID NO:336; 4-23, 27-39, 55-63 and 35-58 of SEQ ID NO:337; 6-26, 28-54 and 28-47 of SEQ ID NO:338; 4-10, 38-52, 58-82 and 30-49 of SEQ ID NO:339; 4-22, 29-35, 44-50, 53-68, 70-80 and 20-33 of SEQ ID NO:340; 22-28, 30-36 and 18-33 of SEQ ID NO:341; 4-11, 13-21, 25-30 and 20-30 of SEQ ID NO:342; 10-22 and 10-23 of SEQ ID NO:343; 4-11 and 9-20 of SEQ ID NO:344; 14-25, 32-46 and 6-19 of SEQ ID NO:345; 5-30 and 14-33 of SEQ ID NO:346; 4-15, 28-35, 46-55, 59-65, 76-84 and 9-24 of SEQ ID NO:347; 27-33 and 5-19 of SEQ ID NO:348; 5-13 and 8-18 of SEQ ID NO:349; 9-22, 24-34 and 21-40 of SEQ ID NO:350; 4-17, 35-41, 46-89, 93-98 and 71-89 of SEQ ID NO:351; 4-12, 14-24 and 2-17 of SEQ ID NO:352; 9-17 and 5-16 of SEQ ID NO:353; 7-41, 48-58, 63-75, 80-89 and 43-53 of SEQ ID NO:354; 4-22, 25-30 and 4-14 of SEQ ID NO:355; 4-55 and 18-33 of SEQ ID NO:356; 262-280 of SEQ ID NO:179; 131-146 of SEQ ID NO:186; 207-224 of SEQ ID NO:188; 27-50, 203-217 and 313-325 of SEQ ID NO:189; 110-129 of SEQ ID NO:192; 156-179, 174-197, 192-215, 210-233, 228-251 and 246-267 of SEQ ID NO:190; 377-400 of SEQ ID NO:196; 34-43, 234-257 and 350-367 of SEQ ID NO:203; 304-327 of SEQ ID NO:207; 25-48, 43-66 and 61-82 of SEQ ID NO:222; 398-421, 416-439, 434-457, 452-475, 470-493, 488-511, 506-529, 524-547, 621-644, 639-664, 707-730, 725-748, 743-766, 761-784, 779-802, 797-820, 984-1007, 1002-1025, 1020-1043, 1038-1061, 1056-1079, 1074-1097, 1092-1115, 1286-1309, 1304-1327, 1322-1345, 1340-1363, 1358-1381, 1376-1399, 1394-1417, 1412-1435, 1430-1453, 1448-1471, 1466-1489 and 1484-1507 of SEQ ID NO:226; 188-211, 206-229, 224-247, 242-265, 260-283 and 278-296 of SEQ ID NO:227; 56-79 and 122-132 of SEQ ID NO:229; 35-46 of SEQ ID NO:231; 178-201, 196-219, 214-237, 232-255, 250-273, 268-291, 379-402, 397-420, 415-438, 433-456, 451-474, 642-665, 660-683, 678-701, 696-719, 714-737, 732-755, 750-773, 768-791, 899-922, 917-940, 935-958, 1037-1060, 1055-1078, 1073-1096 and 1091-1114 of SEQ ID NO:232; 330-346 of SEQ ID NO:233; 571-594, 589-612, 607-630, 625-648, 643-666 and 661-684 of SEQ ID NO:242; 188-207 of SEQ ID NO:244; 61-84, 308-331, 326-349, 344-367, 362-385, 380-403 and 398-421 of SEQ ID NO:249; 79-98, 345-366, 844-867, 870-887 and 890-905 of SEQ ID NO:258; 94-109 of SEQ ID NO:268; 188-207 of SEQ ID NO:272; 290-306 of SEQ ID NO:276; 826-849 of SEQ ID NO:277; 228-252, 247-270, 265-288, 283-306, 301-324, 955-978, 973-996, 991-1014, 1009-1032, 1027-1050, 1045-1068, 2533-2556, 2551-2574, 2569-2592, 2587-2610, 2605-2628 and 2623-2646 of SEQ ID NO:279; 86-109 and 104-127 of SEQ ID NO:288; 546-560 of SEQ ID NO:289; 260-271 of SEQ ID NO:290; 106-129, 124-147, 142-165, 160-183, 178-201 and 375-398 of SEQ ID NO:291; 284-307 of SEQ ID NO:292; 362-385 of SEQ ID NO:301.

41. The hyperimmune serum-reactive antigen or fragment of claim 38, comprising at least 6 contiguous amino acids of any of SEQ ID NOs: 179-356.

42. The hyperimmune serum-reactive antigen or fragment of claim 38, comprising at least 8 contiguous amino acids of any of SEQ ID NOs: 179-356.

43. The hyperimmune serum-reactive antigen or fragment of claim 38, comprising at least 10 contiguous amino acids of any of SEQ ID NOs: 179-356.

44. The hyperimmune serum-reactive antigen or fragment of claim 38, further defined as directed against H. pylori.

45. A pharmaceutical composition comprising a hyperimmune serum-reactive antigen or fragment of claim 38.

46. The pharmaceutical composition of claim 45, wherein the hyperimmune serum-reactive antigen or fragment is directed against H. pylori.

47. The pharmaceutical composition of claim 45, further defined as comprising at least two different hyperimmune serum-reactive antigens and/or fragments.

48. The pharmaceutical composition of claim 47, wherein the at least two different hyperimmune serum-reactive antigens and/or fragments are both directed against H. pylori.

49. The pharmaceutical composition of claim 45, further comprising an immunostimulatory substance.

50. The pharmaceutical composition of claim 49, wherein the immunostimulatory substance is a polycationic polymer, an immunostimulatory deoxynucleotide (ODN), a peptide containing at least two LysLeuLys motifs, a neuroactive compound, alum, or a Freund's complete or incomplete adjuvant.

51. The pharmaceutical composition of claim 50, wherein the polycationic polymer is a polycationic peptide.

52. The pharmaceutical composition of claim 50, wherein the neuroactive compound is human growth hormone.

53. The pharmaceutical composition of claim 45, further defined as a vaccine.

54. The pharmaceutical composition of claim 53, further defined as a vaccine for treatment and/or prevention of H. pylori infection.

55. A method of vaccinating a subject comprising:
obtaining a pharmaceutical composition of claim 45; and
administering the pharmaceutical composition to a subject;
wherein the subject is vaccinated.

56. The method of claim 55, wherein the subject is a human.

57. The method of claim 55, further defined as a method of treating and/or preventing H. pylori infection in the subject.

58. The method of claim 55, wherein the hyperimmune serum-reactive antigen or fragment is directed against H. pylori.

59. The method of claim 55, wherein the pharmaceutical composition comprises at least two different hyperimmune serum-reactive antigens or fragments thereof.

60. The method of claim 59, wherein the at least two different hyperimmune serum-reactive antigens or fragments thereof are both directed against H. pylori.
